SEQUENCE LISTING (5 sequences)

SEQ ID NO: 1 - DNA, 10 bp, synthetic construct
  source 1..10: mol_type = other DNA
ttttgttttt

SEQ ID NO: 2 - DNA, 10 bp, synthetic construct
  source 1..10: mol_type = other DNA
tttgggtttt

SEQ ID NO: 3 - DNA, 10 bp, synthetic construct
  source 1..10: mol_type = other DNA
tgtgtgtttt

SEQ ID NO: 4 - DNA, 10 bp, synthetic construct
  source 1..10: mol_type = other DNA
ttggggggtt

SEQ ID NO: 5 - DNA, 10 bp, synthetic construct
  source 1..10: mol_type = other DNA
tttttttttt

(19) United States
(12) Patent Application Publication
(10) Pub. No.: US 2026/0125413 A1
(43) Pub. Date: May 7, 2026

(54) UNIVERSAL SOLID SUPPORTS AND PHOSPHORAMIDITE BUILDING BLOCKS FOR OLIGONUCLEOTIDE SYNTHESIS

(71) Applicant: AM CHEMICALS LLC, Oceanside, CA (US)

(72) Inventors: Andrei Pavel Guzaev, Escondido, CA (US); Khirud Gogoi, San Marcos, CA (US); Suzie Sheng, Vista, CA (US); Harri Oskari Loennberg, Oceanside, CA (US); Mikhail A. Guzaev, Valley Center, CA (US)

(73) Assignee: AM CHEMICALS LLC, Oceanside, CA (US)

(21) Appl. No.: 19/106,597

(22) PCT Filed: Aug. 28, 2023
(86) PCT No.: PCT/US2023/073030

§ 371 Date: Feb. 25, 2025

Related U.S. Application Data
(60) Provisional application No. 63/373,619, filed on Aug. 26, 2022.

(51) Int. Cl. C07H 21/00; C07D 451/10; C07D 451/12; C07H 1/02

(52) U.S. Cl. CPC C07H 21/00; C07D 451/10; C07D 451/12; C07H 1/02; C07B 2200/11

(57) ABSTRACT

Disclosed herein are chemical preparation of oligonucleotides, chemical entities useful in such preparation, processes for such preparation, and methods of use relating to the chemical preparation of oligonucleotides. Further disclosed herein are novel universal solid supports and phosphoramidite building blocks for oligonucleotide synthesis on solid phase that effect the removal of 3′-phosphate moieties during the course of oligonucleotide deprotection.

### CROSS REFERENCE TO RELATED APPLICATIONS

This application is a national stage application, filed under 35 U.S.C. § 371, of International Patent Application No. PCT/US2023/073030, filed on Aug. 28, 2023, which claims the benefit of U.S. Provisional Patent Application No. 63/373,619, filed on Aug. 26, 2022, each of which is incorporated by reference herein in its entirety.

### REFERENCE TO A “SEQUENCE LISTING,” A TABLE, OR A COMPUTER PROGRAM LISTING APPENDIX SUBMITTED ON A COMPACT DISK

The instant application contains a Sequence Listing which has been submitted via EFS-Web and is hereby incorporated by reference in its entirety. Said XML copy, created on Feb. 26, 2025, is named AM Chemicals-PCT-US.xml and is 8,192 bytes in size.

### FIELD

The disclosure herein provides teaching of compounds, compositions and methods of use relating to synthesis of oligonucleotides. For example, the disclosure provides universal solid supports and phosphoramidite building blocks based on non-nucleosidic linkers for synthesis of standard and modified oligonucleotides, compositions comprising such non-nucleosidic solid supports, phosphoramidite building blocks, and methods of using such supports and building blocks in the synthesis of modified oligonucleotides.

### BACKGROUND

A number of innovations have been introduced to the art of oligonucleotide synthesis. Amongst these innovations have been the development of excellent orthogonal protecting groups, activators, reagents, and synthetic conditions. The oligonucleotides themselves have been subject to a variety of modifications and improvements. Amongst these are chemistries that deliver the properties that are not present in naturally occurring oligonucleotides i.e. reduced negative charge, hydrophobicity, ability to emit fluorescence, protein and receptor binding properties, etc. These novel chemistries generally involve modification of building blocks of non-nucleosidic nature that become the constituent parts of the oligonucleotide.

Oligonucleotides with a free 3′-hydroxy group essential for the enzymatic extension are the ones used most frequently in life sciences. Until late 1990's, the routine synthesis of these oligonucleotides has almost exclusively been carried out on nucleosidic solid supports containing 3′-terminal nucleosides attached via a readily cleavable ester linkage. An alternative approach uses universal solid supports. A universal solid support is one in which the 3′-terminal nucleoside residue is coupled to the support as a phosphoramidite building block in the first cycle of oligonucleotide synthesis. The oligonucleotide chain assembly then continues until the completion, and the support bound material is deprotected. It is important that, over the course of the final deprotection, the phosphate bridge formed between a universal linker and the 3′-terminal nucleoside can be cleaved in such a manner that the phosphate bridge remains with the universal linker. The net result of the deprotection is the oligonucleotide with the free, unprotected 3′-terminal hydroxy group identical to that prepared on nucleosidic solid supports is formed.

The process of deprotection of synthetic oligonucleotides assembled on universal solid supports involves several relatively independent reactions, including: 1) removal of 2-cyanoethyl protecting group from internucleosidic phosphate or phosphorothioate residues; 2) deprotection of amino groups in nucleic bases that, in the standard and the most robust protecting scheme are protected with acetyl or benzoyl groups for cytidine (C^{ac} and C^{bz}, respectively), benzoyl group for adenosine (A^{bz}), isobutyryl group for guanosine (G^{ib}) and for their 2′-deoxy, 2′-O-alky, and other analogs used in the art; 3) release of solid support-bound oligonucleotide in solution, and 4) 3′-dephosphorylation of the 3′-hydroxy group attached, via a phosphate or a phosphorothioate linkage to the universal linker.

Of these processes, the removal of the isobutyryl group from G^{ib} residues and 3′-dephosphorylation are the most time consuming and hence controls the length of the deprotection time, and has been reported as the rate limiting step (Schwartz, M. E., Breaker, R. R., Asteriadis, G. T., and Gough, G. R. 1995)

Analysis of the prior art reveals that all universal solid supports disclosed to date share one common disadvantage in that the 3′-dephosphorylation of oligonucleotides synthesized on said solid supports remains the rate-limiting step in the course of deprotection of oligonucleotides.

### SUMMARY OF THE INVENTION

One object of this disclosure is to provide novel compounds which may serve as solid supports and phosphoramidite building blocks for preparation of oligomeric compounds, analogs of natural and chemically modified oligonucleotides, wherein a non-nucleosidic moiety together with the phosphate moiety it is attached to is cleaved from the target oligonucleotides during the course of the final deprotection thereby releasing the free 3′-hydroxy group in said oligonucleotide.

Disclosed herein are compounds of Formula I

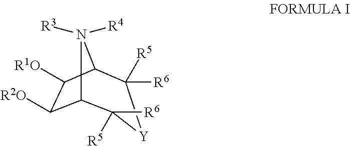

wherein:
R¹ and R² either form an orthoester function —C(CH₃)(OCH₃)— or one of R¹ and R² is hydrogen, a trityl-type protecting group, or a xanthenyl-type protecting group, and the other of R¹ and R² is an acetyl, a propionyl, a n-butyryl, a benzoyl, or L¹, wherein:
   L¹ is a linking moiety —C(═O)—Z—(C═O)-A¹, wherein:
      Z is selected from the group consisting of a covalent bond, a methylene group, —(CH₂)₂—, —(CH₂)—O—(CH₂)—, and —(CH₂)—O—C₆H₄—O—(CH₂)—;
      A¹ is a hydroxy group, a salt of a hydroxy group and an inorganic cation or a tertiary amine, a covalent bond to SP¹, or —NH(CH₂)ₙ—OR⁷, wherein:
         SP¹ is an oxygen, amino, an aminoalkyl or a hydroxyalkyl covalently attached to a solid phase material comprising a controlled pore glass, a magnetic controlled pore glass, a silica-containing particle, a styrene-containing polymer or copolymer, a divinylbenzene-containing polymer or copolymer, a copolymer of styrene and divinylbenzene, a controlled pore glass grafted with a styrene-containing polymer, a controlled pore glass grafted with a copolymer of styrene and divinylbenzene, a copolymer of styrene and divinylbenzene grafted with polyethylene glycol, or a flat glass surface;
         n is an integer from 2 to 10;
         R⁷ is hydrogen or PA, wherein:

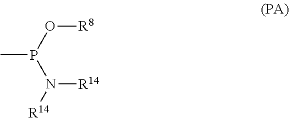

and wherein R⁸ is methyl or 2-cyanoethyl group and R¹⁴ is an alkyl, iso-alkyl, sec-alkyl, or tert-alkyl;
R³ is selected from the group consisting of methyl, ethyl, propyl, isopropyl, and benzyl;
R⁴ is selected from the group consisting of a lone electron pair, hydrogen, methyl, ethyl, propyl, isopropyl, and benzyl, wherein when R⁴ is other than a lone electron pair, N has a positive charge and forms a salt with a halide anion or with an intramolecular carboxylic functional group;
R⁵ and R⁶ are independently hydrogen or methyl;
Y is —(C═O)—, —CH(OR⁹)—, —CH(NR¹⁰R¹¹)—, or —[C(OR¹²)(OR¹³)]—, wherein:
   R⁹ is hydrogen, methyl, ethyl, benzyl, acetyl, propionyl, butyryl, isobutyryl, pivaloyl, benzoyl, t-butyldimethylsilyl, triisopropylsilyl, (t-butyl) diphenylsilyl, L¹, PA, or —(CH₂)ₘ—O—PA, wherein:
      m is an integer ranging from 2 to 10;
   R¹⁰ is L¹, —(C═O)-A¹, or —(C═O)-W¹-(CH₂)ₚ—W², wherein:
      W¹ is —(CH₂)—, —(NH)—, or —(NH)—(C═O)—;
      W² is a hydroxy, amino, —O-PA, —(C═O)-A¹, or —[NH(C═O)]-A¹; and
      p is an integer from 2 to 10;
   R¹¹ is hydrogen, methyl, ethyl, or benzyl; and
   R¹² and R₁₃ together form ketal bridges —(CH₂)₂— or —CH₂—[C(CH₃)₂]—CH₂—;
wherein only one of R¹, R², and Y may be or include L¹ or PA.

Another object of this disclosure is to provide methods for synthetic preparation of said universal linkers, for their attachment to solid phase materials, and for their use in oligonucleotide synthesis. Accordingly, also disclosed herein are methods for functionalizing a solid phase material with a first monomeric subunit, comprising the steps of:
(a) providing a solid phase material-bound compound of Formula I

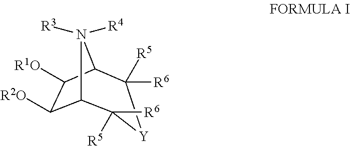

wherein:
R¹ and R² either form an orthoester function —C(CH₃)(OCH₃)— or one of R¹ and R² is hydrogen, a trityl-type protecting group, or a xanthenyl-type protecting group and the other of R¹ and R² is an acetyl, a propionyl, a n-butyryl, a benzoyl, L¹, wherein:
   L¹ is a linking moiety —C(═O)-Z-(C═O)-A¹, wherein:
      Z is selected from the group consisting of a covalent bond, a methylene group, —(CH₂)₂—, —(CH₂)—O—(CH₂)—, and —(CH₂)—O—C₆H₄—O—(CH₂)—;
      A¹ is a covalent bond to SP¹ or —NH(CH₂)ₙ—OR⁷, wherein:
         SP¹ is an oxygen, amino, an aminoalkyl or a hydroxyalkyl covalently attached to a solid phase material comprising a controlled pore glass, a magnetic controlled pore glass, a silica-containing particle, a styrene-containing polymer or copolymer, a divinylbenzene-containing polymer or copolymer, a copolymer of styrene and divinylbenzene, a controlled pore glass grafted with a styrene-containing polymer, a controlled pore glass grafted with a copolymer of styrene and divinylbenzene, a copolymer of styrene and divinylbenzene grafted with polyethylene glycol, and a flat glass surface;
         n is an integer from 2 to 10;
         R⁷ is PX, wherein:

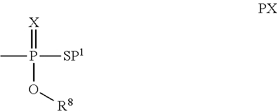

and wherein R⁸ is methyl or 2-cyanoethyl group and X is oxygen or sulfur;
R³ is selected from the group consisting of methyl, ethyl, propyl, isopropyl, and benzyl;
R⁴ is selected from the group consisting of a lone electron pair, hydrogen, methyl, ethyl, propyl, isopropyl, and benzyl, wherein when R⁴ is other than a lone electron pair, N has a positive charge and forms a salt with a halide anion or with an intramolecular carboxylic functional group;
R⁵ and R⁶ are independently hydrogen or methyl;
Y is —(C═O)—, —CH(OR⁹)—, —CH(NR¹⁰R¹¹)—, or —[C(OR¹²)(OR¹³)]—, wherein:
   R⁹ is hydrogen, methyl, ethyl, benzyl, acetyl, propionyl, butyryl, isobutyryl, pivaloyl, benzoyl, t-butyldimethylsilyl, triisopropylsilyl, or (t-butyl) diphenylsilyl group, L¹, PX, or —(CH₂)ₘ—O-PX, wherein:
      m is an integer ranging from 2 to 10;
   R¹⁰ is L¹, —(C═O)-A¹, or —(C═O)-W¹-(CH₂)ₚ—W², wherein:
      W¹ is —(CH₂)—, —(NH)—, or —(NH)—(C═O)—;
      W² is —O-PX, or —[NH(C═O)]-A¹; and
      p is an integer ranging from 2 to 10;
   R¹¹ is hydrogen, methyl, ethyl, or benzyl; and
   R¹² and R₁₃ together form ketal bridges —(CH₂)₂— or —CH₂—[C(CH₃)₂]—CH₂;
wherein only one of R¹, R², and Y may be or include L¹ or PX;
(b) selectively removing one of the protecting groups of Formula I to form a reactive hydroxyl group;
(c) providing a first monomeric subunit comprising an activated phosphorus group and a protected hydroxy group, and reacting the activated phosphorus group of the first monomeric subunit with the reactive hydroxyl group of the compound of Formula I to form a monomer-functionalized solid support comprising a phosphite group;
(d) treating said monomer-functionalized solid support with a capping agent and/or treating said monomer-functionalized solid support with an oxidizing solution or a sulfurizing agent to convert the phosphite triester group to a phosphotriester or phosphothioate triester, thereby forming an oxidized or sulfurized functionalized solid support;
(e) optionally repeating steps (b), (c), and (d) one or more times for the oxidized or sulfurized functionalized solid support to form an oligomeric-functionalized solid support, wherein the monomeric subunit is the same or different each time steps (b), (c), and (d) are repeated.

### BRIEF DESCRIPTION OF THE DRAWINGS

FIG. 1 shows a synthetic scheme for the preparation of starting materials.

FIG. 2 shows a synthetic scheme for the preparation of universal solid supports wherein the 7-O-DMT-3-O-acyl or -3-O-silyl- and 7-O-TMT-3-O-acyl or -3-O-silyl protected, universal linkers are attached to the solid-phase material via the 6-O-position.

FIG. 3 shows a synthetic scheme for the preparation of the 7-O-TMT-protected and 7-O-deprotected universal solid supports wherein the respective 3-O-alkylated universal linkers are attached to the solid-phase material via the 6-O-position and following removal of the TMT group.

FIG. 4 shows a synthetic scheme for the preparation of universal solid supports wherein the 7-O-TMT-protected [3, 2′]spirodioxolane and [3, 2′]spirodioxane universal linkers are attached to the solid-phase material via the 6-O-position.

FIG. 5 shows a synthetic scheme for the preparation of universal solid supports wherein the 7-O-DMT and 7-O-TMT-protected 6-O-acetylated universal linkers are attached to the solid-phase material via the 3-N-position.

FIG. 6 shows a synthetic scheme for the preparation of universal solid supports wherein the 7-O-DMT and 7-O-TMT-protected, 6-O-acetylated universal linkers are attached to the solid-phase material via the 3-O-position.

FIG. 7 shows a synthetic scheme for the preparation of 7-O-DMT and 7-O-TMT-protected universal phosphoramidites wherein the phosphoramidite moiety is attached to the universal linker via the 3-O- or 6-O-position of said linker.

FIG. 8 shows a synthetic scheme for the preparation of 7-O-TMT-protected universal phosphoramidites wherein the phosphoramidite moiety is attached to the universal linker via the 3-N-position of said linker.

FIG. 9 shows a synthetic scheme for the preparation of 6,7-orthoacetate-protected universal phosphoramidites wherein the phosphoramidite moiety is attached to the universal linker via the 3-O- or 3-N-position of said linker.

FIG. 10 shows DMT-protected solid supports with the tertiary amino group.

FIG. 11 shows TMT-protected solid supports with the tertiary amino group.

FIG. 12 shows DMT-protected universal solid supports.

FIG. 13 shows TMT-protected universal solid supports.

FIG. 14 shows unprotected universal solid supports.

FIG. 15 shows a reversed-phase HPLC profile of oligonucleotide 40 synthesized on universal solid support 941c.

FIG. 16 shows time course of 3′-dephosphorylation of 40 by universal solid supports 941 in 2.56 M MeNH₂ at 45° C.

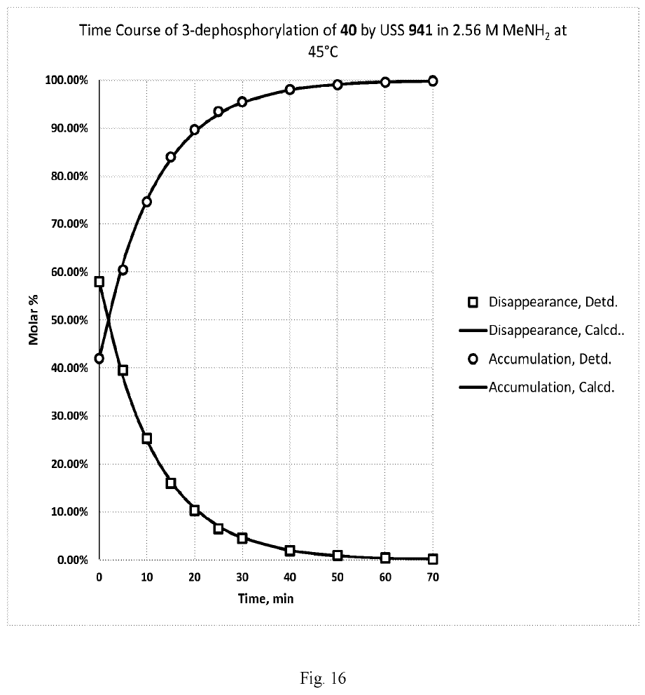

FIG. 17 shows a plot of the dependence of the pseudo-first order rate constant k for dephosphorylation of compound 41 assembled on universal solid supports 941c and 943c on the concentration of aqueous methylamine at 45° C.

FIG. 18 shows a plot of the time course of deprotection of T₂dG^{ib}₆T₂ (45) in 3.42 M aqueous methylamine at 40° C.

FIG. 19 shows a plot wherein the time required for dephosphorylation of oligonucleotide 41 synthesized on universal solid support 941c (solid line) and that for the deprotection of the N-isobutyryl guanosine bases in oligonucleotide 45 (dotted line), each to 99% extent (t_{99%}), is plotted against the concentration of aqueous methylamine at 45° C.

### DETAILED DESCRIPTION

The invention relates to 1) the synthesis of compounds to facilitate oligonucleotide synthesis, 2) chemical preparation of oligonucleotides, 3) chemical entities useful in such preparation, 4) processes for such preparation, and 5) methods of use relating to the chemical preparation of oligonucleotides. Specifically, the invention provides novel non-nucleosidic phosphoramidite building blocks and solid supports for incorporation of a variety of useful ligands to natural oligonucleotides and their phosphorothioate analogs in the course of synthesis on solid phase. More specifically, the invention provides novel universal solid supports and phosphoramidite building blocks for oligonucleotide synthesis on solid phase that effect the removal of the 3′-phosphate moieties during the course of oligonucleotide deprotection. The phosphoramidite building blocks and solid supports according to the invention are highly efficient. These compounds are inexpensive in manufacturing. They are stable in the solid state or in solution over an extensive period of time. Their attachment to oligonucleotides does not create any new chiral centers and hence does not complicate the isolation of the ligand-modified oligonucleotides. Said oligonucleotides do not suffer from any unwanted side reactions. The patents and publications cited in this specification are hereby incorporated by reference in their entirety.

To improve the kinetics of the 3′-dephosphorylation of oligonucleotides assembled on universal solid supports, a novel structural motif of the universal linker that facilitates such 3′-dephosphorylation is required. A set of novel universal linkers for oligonucleotide synthesis, solid supports and phosphoramidite building blocks derived from such linkers, and methods of their preparation are disclosed in the present invention. Also disclosed are the methods for synthesizing the novel structural motifs of the universal linkers for the 3′-dephosphorylation of oligonucleotides effected by said linkers as compared with the kinetic data for deprotection of N-isobutyryl-2′-deoxyguanosine residue in synthetic oligonucleotides, and an optimized deprotection protocol for synthetic oligonucleotides.

### ABBREVIATIONS

Ac: Acetyl;
Bn: benzyl;
Bz, bz: benzoyl;
CDI: carbonyldiimidazole;
DCM: dichloromethane;
CPG: controlled pore glass;
DIPEA: N-ethyl-N,N-diisopropylamine;
DMF: N,N-dimethylformamide;
DMT: bis(4-methoxyphenyl)phenylmethyl (4,4′-dimethoxytrityl);
ES MS: mass-spectrometry with electron-spray ionization;
Et: ethyl;
Fmoc: (9-fluorenyl)methyloxycarbonyl;
HPLC: high-performance liquid chromatography;
ib: isobutyryl;
iPr: isopropyl;
Me: methyl;
MeCN: acetonitrile;
MPPS: microporous polystyrene;
NMI: N-methylimidazol;
PTSA: p-toluenesulfonic acid;
Py: pyridine;
TBTU: [O-(Benzotriazol-1-yl)-N,N,N′,N′-tetramethyluronium tetrafluoroborate];
TEA: triethylamine;
TCA: trichloroacetic acid;
TFA: trifluoroacetic acid;
TLC: thin-layer chromatography;
TMT: tris(4-methoxyphenyl)methyl (4,4′,4″-trimethoxytrityl);
USS: universal solid support.

### Compounds of Formula I for Oligonucleotide Synthesis

Disclosed herein are compounds of Formula I

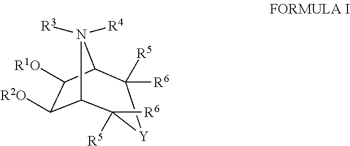

wherein:
R¹ and R² either form an orthoester function —C(CH₃)(OCH₃)— or one of R¹ and R² is hydrogen, a trityl-type protecting group, or a xanthenyl-type protecting group, and the other of R¹ and R² is an acetyl, a propionyl, a n-butyryl, a benzoyl, or L¹, wherein:
   L¹ is a linking moiety —C(═O)—Z—(C═O)-A¹, wherein:
      Z is selected from the group consisting of a covalent bond, a methylene group, —(CH₂)₂—, —(CH₂)—O—(CH₂)—, and —(CH₂)—O—C₆H₄—O—(CH₂)—;
      A¹ is a hydroxy group, a salt of a hydroxy group and an inorganic cation or a tertiary amine, a covalent bond to SP¹, or —NH(CH₂)ₙ—OR⁷, wherein:
         SP¹ is an oxygen, amino, an aminoalkyl or a hydroxyalkyl covalently attached to a solid phase material comprising a controlled pore glass, a magnetic controlled pore glass, a silica-containing particle, a styrene-containing polymer or copolymer, a divinylbenzene-containing polymer or copolymer, a copolymer of styrene and divinylbenzene, a controlled pore glass grafted with a styrene-containing polymer, a controlled pore glass grafted with a copolymer of styrene and divinylbenzene, a copolymer of styrene and divinylbenzene grafted with polyethylene glycol, or a flat glass surface;
         n is an integer from 2 to 10;
         R⁷ is hydrogen or PA, wherein:

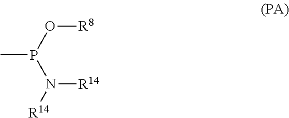

and wherein R⁸ is methyl or 2-cyanoethyl group and R¹⁴ is an alkyl, iso-alkyl, sec-alkyl, or tert-alkyl;
R³ is selected from the group consisting of methyl, ethyl, propyl, isopropyl, and benzyl;
R⁴ is selected from the group consisting of a lone electron pair, hydrogen, methyl, ethyl, propyl, isopropyl, and benzyl, wherein when R⁴ is other than a lone electron pair, N has a positive charge and forms a salt with a halide anion or with an intramolecular carboxylic functional group;
R⁵ and R⁶ are independently hydrogen or methyl;
Y is —(C═O)—, —CH(OR⁹)—, —CH(NR¹⁰R¹¹)—, or —[C(OR¹²)(OR¹³)]—, wherein:
   R⁹ is hydrogen, methyl, ethyl, benzyl, acetyl, propionyl, butyryl, isobutyryl, pivaloyl, benzoyl, t-butyldimethylsilyl, triisopropylsilyl, (t-butyl) diphenylsilyl, L¹, PA, or —(CH₂)ₘ—O-PA, wherein:
      m is an integer ranging from 2 to 10;
   R¹⁰ is L¹, —(C═O)-A¹, or —(C═O)-W¹-(CH₂)ₚ—W², wherein:
      W¹ is —(CH₂)—, —(NH)—, or —(NH)—(C═O)—;
      W² is a hydroxy, amino, —O-PA, —(C═O)-A¹, or —[NH(C═O)]-A¹; and
      p is an integer from 2 to 10;
   R¹¹ is hydrogen, methyl, ethyl, or benzyl; and
   R¹² and R₁₃ together form ketal bridges —(CH₂)₂— or —CH₂—[C(CH₃)₂]—CH₂—;
wherein only one of R¹, R², and Y may be or include L¹ or PA.

In some examples of the compound of Formula I, one of R¹ and R² is hydroxy, tris-(4-methoxyphenyl)methyl, bis-(4-methoxyphenyl)phenylmethyl, 9-phenylxanthen-9-yl, or 9-(4-methoxyphenyl)xanthen-9-yl, and the other of R¹ and R² is L¹. In some examples where one of R¹ and R² is L¹, A¹ is a hydroxy group, a salt of a hydroxy group and an inorganic cation or a tertiary amine, or a covalent bond to SP¹. In some examples, Y is-CH(OR⁹)—, wherein R⁹ is methyl, ethyl, benzyl, acetyl, propionyl, butyryl, isobutyryl, pivaloyl, benzoyl, t-butyldimethylsilyl, triisopropylsilyl, or phenyldimethylsilyl. In some examples, Y is —[C(OR¹²)(OR¹³)]—.

In some examples of the compound of Formula I, one of R¹ and R² is hydroxy, tris-(4-methoxyphenyl)methyl, or bis-(4-methoxyphenyl)phenylmethyl, and the other of R¹ and R² is acetyl, propionyl, n-butyryl, or benzoyl. In some examples, Y is —CH(OR⁹)—, wherein R⁹ is L¹, and wherein A¹ is a hydroxy group optionally forming a salt with an inorganic cation or a tertiary amine, or optionally forming a covalent bond to SP¹. In other examples, Y is —CH(NR¹⁰R¹¹)—, wherein R¹¹ is hydrogen, methyl, ethyl, or benzyl, and R¹⁰ is L¹ wherein A¹ is a hydroxy group, a salt of a hydroxy group and an inorganic cation or a tertiary amine, or a covalent bond to SP¹. In still other examples, Y is —CH(NR¹⁰R¹¹)—, wherein R¹¹ is hydrogen, methyl, ethyl, or benzyl, and R¹⁰ is (C═O)-W¹-(CH₂)ₚ—W², wherein W² is an amino, —(C═O)-A¹, or —[NH(C═O)]-A¹ A¹ is a hydroxy group, a salt of a hydroxy group and an inorganic cation or a tertiary amine, or a covalent bond to SP¹, and optionally p is an integer from 3 to 10.

In some examples of the compound of Formula I, one of R¹ and R² is tris-(4-methoxyphenyl)methyl or bis-(4-methoxyphenyl)phenylmethyl and the other of R¹ and R² is L¹. In some examples, A¹ is —NH(CH₂), OR⁷ and Y is —CH(OR⁹). In some examples, R⁹ is methyl, ethyl, benzyl, acetyl, propionyl, butyryl, isobutyryl, pivaloyl, benzoyl, t-butyldimethylsilyl, triisopropylsilyl, or phenyldimethylsilyl.

In some examples of the compound of Formula I, one of R¹ and R² is tris-(4-methoxyphenyl)methyl or bis-(4-methoxyphenyl)phenylmethyl and the other of R¹ and R² is acetyl, propionyl, n-butyryl, or benzoyl and Y is —CH(OR⁹)—. In some examples, R⁹ is PA. In some examples, R⁹ is —(CH₂)ₘ—O-PA.

In some examples of the compound of Formula I, one of R¹ and R² is tris-(4-methoxyphenyl)methyl or bis-(4-methoxyphenyl)phenylmethyl, the other of R¹ and R² is acetyl, propionyl, n-butyryl, or benzoyl, and Y is —CH(NR¹⁰R¹¹)—, wherein R¹¹ is hydrogen, methyl, ethyl, or benzyl. In some examples, R¹⁰ is —(C═O)-W¹-(CH₂)ₚ—W². In some examples W¹ is —(CH₂), —(NH)—, or —(NH)—(C═O)—, and W² is hydroxy or O-PA.

In some examples of the compound of Formula I, R¹ and R² form an orthoester function —C(CH₃)(OCH₃)—, wherein Y is —CH(OR⁹)— and R⁹ is hydrogen, PA, or —(CH₂)ₘ—O—PA.

In some examples of the compound of Formula I, wherein the trityl-type protecting group or the xanthenyl-type protecting group is selected from the group consisting of 4-methoxytrityl, 4,4′-dimethoxytrityl, 4,4′,4″-trimethoxytrityl, 9-phenylxanthen-9-yl, and 9-(4-methoxyphenyl)xanthen-9-yl.

In some examples, Z is selected from the group consisting of a covalent bond, a methylene group, —(CH₂)₂—, —(CH₂)—O—(CH₂)—, and —(CH₂)—O—C₆H₄—O—(CH₂), a substituted or unsubstituted alkyl, a substituted or unsubstituted alkene, a substituted or unsubstituted alkenyl, a substituted or unsubstituted cycloalkyl or a substituted or unsubstituted aryl.

In some examples of the compound of Formula I, one of R¹ and R² is hydroxy, tris-(4-methoxyphenyl)methyl, bis-(4-methoxyphenyl)phenylmethyl, 9-phenylxanthen-9-yl, or 9-(4-methoxyphenyl)xanthen-9-yl; the other of R¹ and R² is L¹; A¹ is a hydroxy group, a salt of a hydroxy group and an inorganic cation or a tertiary amine, or a covalent bond to SP¹; and Y is —CH(OR⁹)—, wherein R⁹ is methyl, ethyl, benzyl, acetyl, propionyl, butyryl, isobutyryl, pivaloyl, benzoyl, t-butyldimethylsilyl, triisopropylsilyl, or phenyldimethylsilyl. In other examples, one of R¹ and R² is hydroxy, tris-(4-methoxyphenyl)methyl, bis-(4-methoxyphenyl)phenylmethyl, 9-phenylxanthen-9-yl, or 9-(4-methoxyphenyl)xanthen-9-yl; the other of R¹ and R² is L¹; A¹ is a hydroxy group, a salt of a hydroxy group and an inorganic cation or a tertiary amine, or a covalent bond to SP¹; and Y is —[C(OR¹²)(OR¹³)]—.

In some examples of the compound of Formula I, one of R¹ and R² is hydroxy, tris-(4-methoxyphenyl)methyl, bis-(4-methoxyphenyl)phenylmethyl, 9-phenylxanthen-9-yl, or 9-(4-methoxyphenyl)xanthen-9-yl; the other of R¹ and R² is L¹; A¹ is a hydroxy group, a salt of a hydroxy group and an inorganic cation or a tertiary amine, or a covalent bond to SP¹; and Y is —CH(NR¹⁰R¹¹)—, wherein R¹⁰ is —(C═O)-W¹-(CH₂)ₚ—W², wherein W¹ is —(CH₂)—, —(NH)—, or —(NH)—(C═O)—; W² is a hydroxy, amino, or —O—PA, and p is an integer from 3 to 10; and R¹¹ is hydrogen, methyl, ethyl, or benzyl. In other examples, one of R¹ and R² is hydroxy, tris-(4-methoxyphenyl)methyl, bis-(4-methoxyphenyl)phenylmethyl, 9-phenylxanthen-9-yl, or 9-(4-methoxyphenyl)xanthen-9-yl; the other of R¹ and R² is L¹; A¹ is a hydroxy group, a salt of a hydroxy group and an inorganic cation or a tertiary amine, or a covalent bond to SP¹; and Y is —(C═O)—.

In some examples of the compound of Formula I, one of R¹ and R² is hydroxy, tris-(4-methoxyphenyl)methyl, or bis-(4-methoxyphenyl)phenylmethyl; the other of R¹ and R² is acetyl, propionyl, n-butyryl, or benzoyl; and Y is —CH(OR⁹)—, wherein R⁹ is L¹, and A¹ is a hydroxy group optionally forming a salt with an inorganic cation or a tertiary amine, or optionally forming a covalent bond to SP¹. In other examples, one of R¹ and R² is hydroxy, tris-(4-methoxyphenyl)methyl, or bis-(4-methoxyphenyl)phenylmethyl, the other of R¹ and R² is acetyl, propionyl, n-butyryl, or benzoyl, and Y is —CH(NR¹⁰R¹¹)—, wherein R¹¹ is hydrogen, methyl, ethyl, or benzyl, and R¹⁰ is L¹, wherein A¹ is a hydroxy group, a salt of a hydroxy group and an inorganic cation or a tertiary amine, or a covalent bond to SP¹. In yet other examples, one of R¹ and R² is hydroxy, tris-(4-methoxyphenyl)methyl, or bis-(4-methoxyphenyl)phenylmethyl, the other of R¹ and R² is acetyl, propionyl, n-butyryl, or benzoyl, and Y is —CH(NR¹⁰R¹¹)—, wherein R¹⁰ is —(C═O)-W¹ (CH₂)ₚ—W², wherein W¹ is —(CH₂)—, —(NH)—, or —(NH)—(C═O)—; W² is a hydroxy, amino, —O-PA, —(C═O)-A¹, or —[NH(C═O)]-A¹; and p is an integer from 3 to 10; wherein R¹¹ is hydrogen, methyl, ethyl, or benzyl. In examples wherein R¹⁰ is —(C═O)-W¹-(CH₂)ₚ—W² and W² is —(C═O)-A¹ or —[NH(C═O)]-A¹, A¹ is a covalent bond to SP¹.

In some embodiments, the compound of Formula I comprises a compound of Table 1:

TABLE 1
TMT-protected solid supports, DMT-protected solid supports unprotected solid supports, and other compounds of Formula I, wherein
indexes ‘c’ and ‘p’ refer, respectively, to CPG and MPPS used as solid phase material.

| 700 Moieties | 800 Moieties | 900 Moieties |
|---|---|---|
| 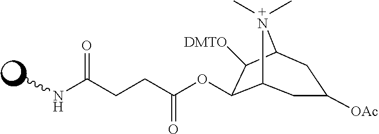 | 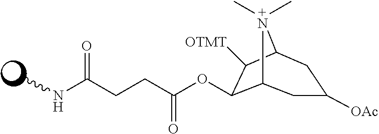 | 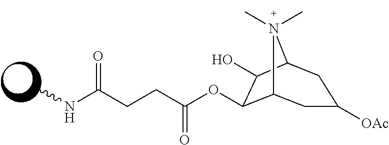 |
| R₃ = R⁴ = CH₃ | R₃ = R⁴ = CH₃ | R₃ = R⁴ = CH₃ |
| 701c, 701p | 801c, 801p | 901c, 901p |
| 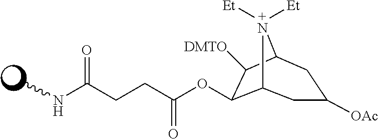 | 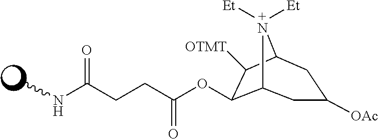 | 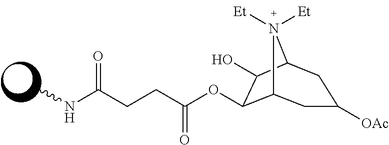 |
| R₃ = R⁴ = Et | R₃ = R⁴ = Et | R₃ = R⁴ = Et |
| 702c, 702p | 802c, 802p | 902c, 902p |
| 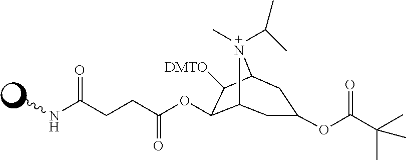 | 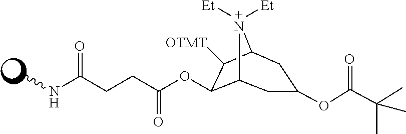 | 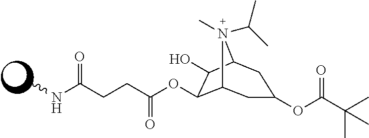 |
| R₃ = Me R⁴ = iPr | R₃ = Me R⁴ = iPr | R₃ = Me R⁴ = iPr |
| 703c, 703p | 803c, 803p | 903c, 903p |
| 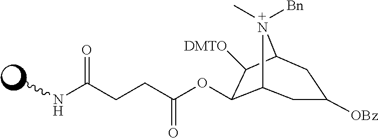 | 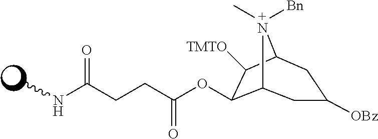 | 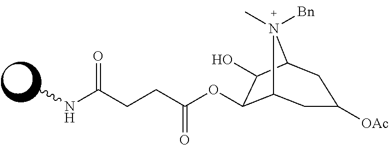 |
| R₃ = Me R⁴ = Bn | R₃ = Me R⁴ = Bn | R₃ = Me R⁴ = Bn |
| 704c, 704p | 804c, 804p | 904c, 904p |
| 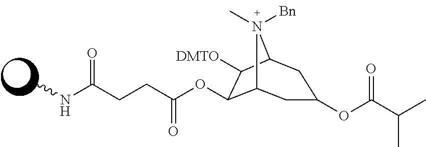 | 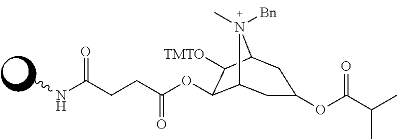 | 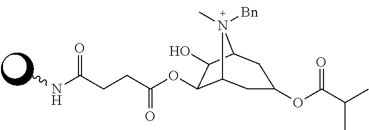 |
| R₃ = Me R⁴ = Bn | R₃ = Me R⁴ = Bn | R₃ = Me R⁴ = Bn |
| 705c, 705p | 805c, 805p | 905c, 905p |
| 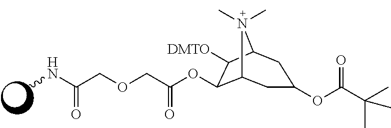 | 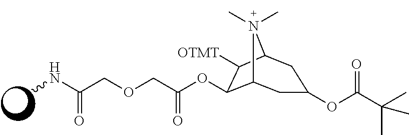 | |
| R₃ = R⁴ = CH₃ | R₃ = R⁴ = CH₃ | |
| 706c, 706p | 806c, 806p | |
| 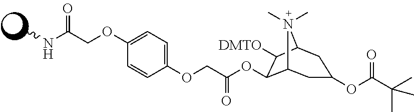 | 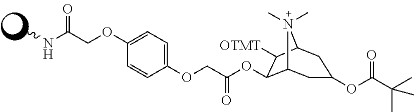 | |
| R₃ = R⁴ = CH₃ | R₃ = R⁴ = CH₃ | |
| 707c, 707p | 807c, 807p | |
| 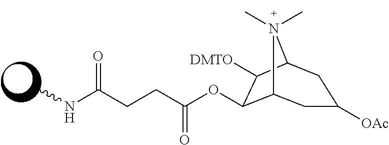 | 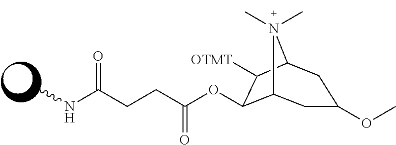 | 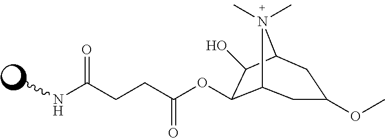 |
| 711c, 711p | R₃ = R⁴ = CH₃ | R₃ = R⁴ = CH₃ |
| | 811c, 811p | 911c, 911p |
| 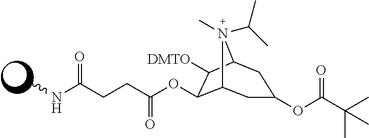 | 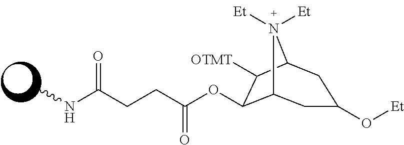 | 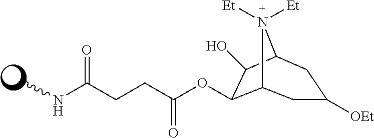 |
| 712c, 712p | R₃ = R⁴ = Et | R₃ = R⁴ = Et |
| | 812c, 812p | 912c, 912p |
| 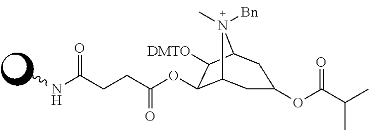 | 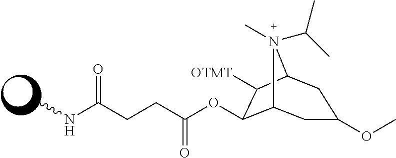 | 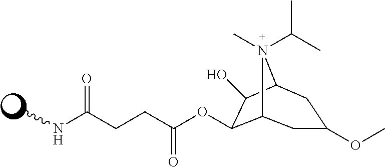 |
| 713c, 713p | R₃ = Me R⁴ = iPr | R₃ = Me R⁴ = iPr |
| | 813c, 813p | 913c, 913p |
| | 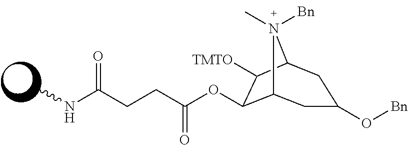 | 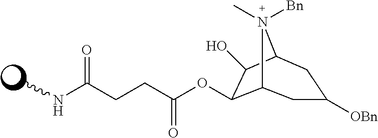 |
| | R₃ = Me R⁴ = Bn | R₃ = Me R⁴ = Bn |
| | 814c, 814p | 914c, 914p |
| | 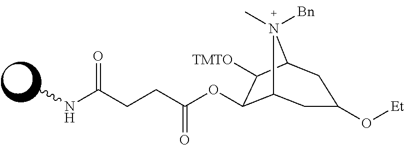 | 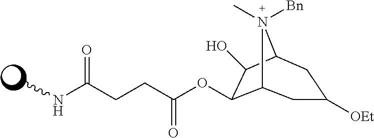 |
| | R₃ = Me R⁴ = Bn | R₃ = Me R⁴ = Bn |
| | 815c, 815p | 915c, 915p |
| | 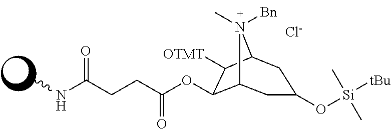 | 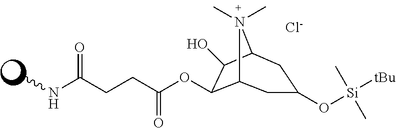 |
| | R₃ = R⁴ = CH₃ | R₃ = R⁴ = CH₃ |
| | 816c, 816p | 916c, 916p |
| | 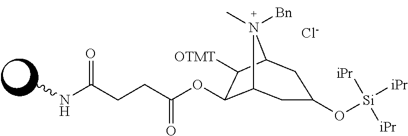 | 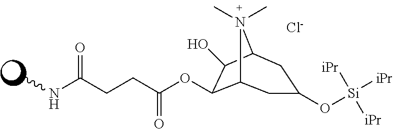 |
| | R₃ = R⁴ = CH₃ | R₃ = R⁴ = CH₃ |
| | 817c, 817p | 917c, 917p |
| | 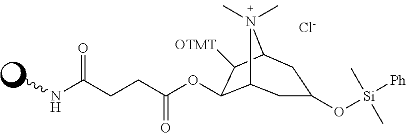 | 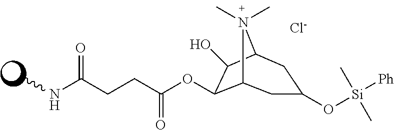 |
| | R₃ = R⁴ = CH₃ | R₃ = R⁴ = CH₃ |
| | 818c, 818p | 918c, 918p |
| | 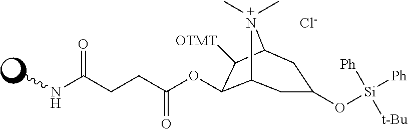 | 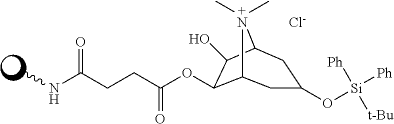 |
| | 819c, 819p | 919c, 919p |
| 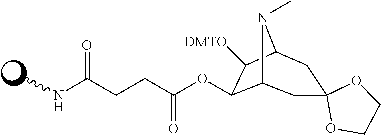 | 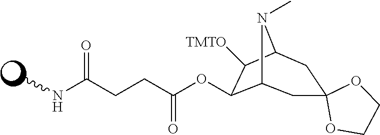 | |
| R₃ = R⁴ = CH₃ | R₃ = CH₃ | |
| 721c, 721p | 821c, 821p | |
| 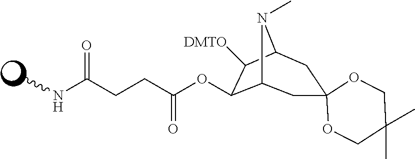 | 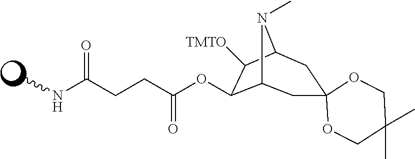 | |
| R₃ = R⁴ = Et | R₃ = CH₃ | |
| 722c, 722p | 822c, 822p | |
| 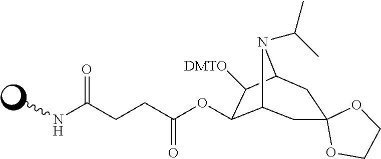 | 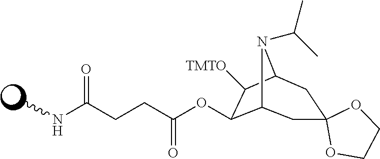 | |
| R₃ = Me R⁴ = iPr | R³ = iPr | |
| 723c, 723p | 823c, 823p | |
| 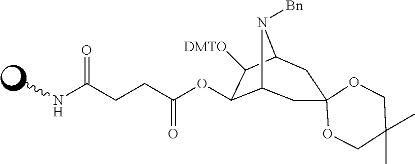 | 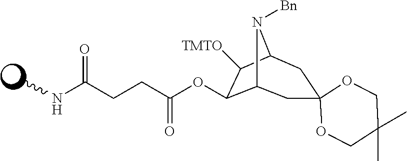 | |
| R₃ = Me R⁴ = Bn | R³ = Bn | |
| 724c, 724p | 824c, 824p | |
| 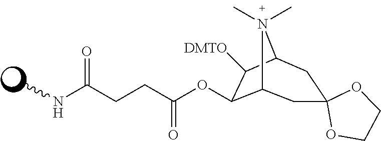 | 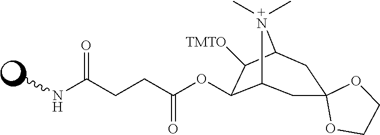 | 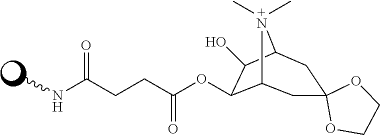 |
| R₃ = R⁴ = CH₃ | R₃ = R⁴ = CH₃ | R₃ = R⁴ = CH₃ |
| 725c, 725p | 825c, 825p | 925c, 925p |
| 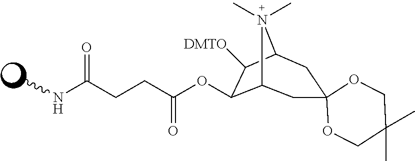 | 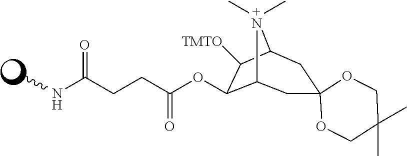 | 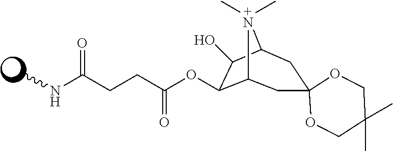 |
| R₃ = R⁴ = CH₃ | R₃ = R⁴ = CH₃ | R₃ = R⁴ = CH₃ |
| 726c, 726p | 826c, 826p | 926c, 926p |
| 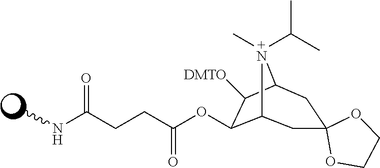 | 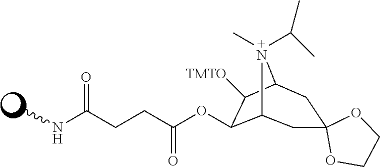 | 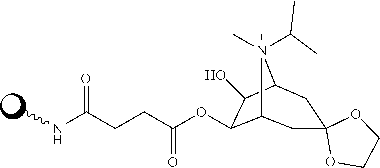 |
| R₃ = Me R⁴ = iPr | R₃ = Me R⁴ = iPr | R₃ = Me R⁴ = iPr |
| 727c, 727p | 827c, 827p | 927c, 927p |
| 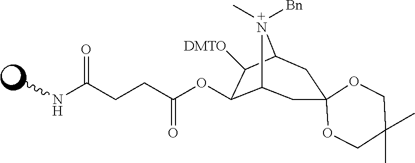 | 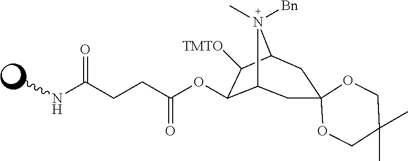 | 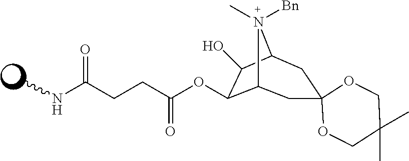 |
| R³ = Me R⁴ = Bn | R₃ = Me R⁴ = Bn | R₃ = Me R⁴ = Bn |
| 728c, 728p | 828c, 828p | 928c, 928p |
| 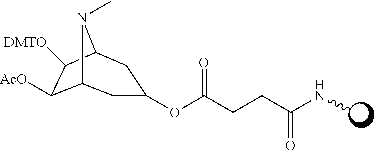 | 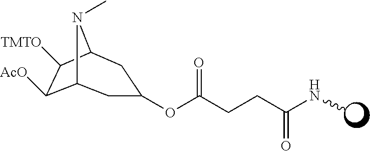 | 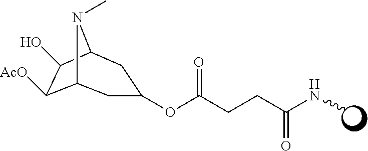 |
| R = CH₃ | R₃ = R⁴ = CH₃ | R = CH₃ |
| 731c, 731p | 831c, 831p | 931c, 931p |
| 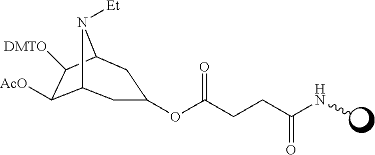 | 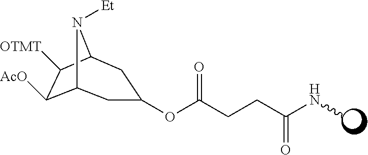 | 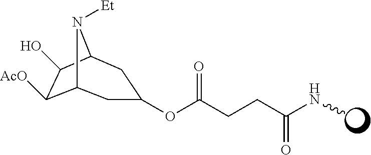 |
| R = Et | R₃ = R⁴ = Et | R = Et |
| 732c, 732p | 832c, 832p | 932c, 932p |
| 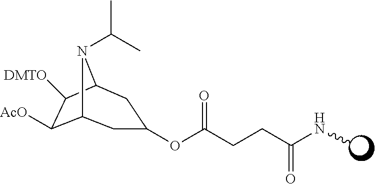 | 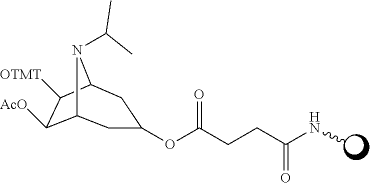 | 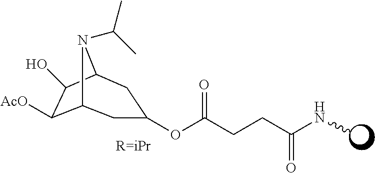 |
| R = iPr | R₃ = Me, R⁴ = iPr | R = iPr |
| 733c, 733p | 833c, 833p | 933c, 933p |
| 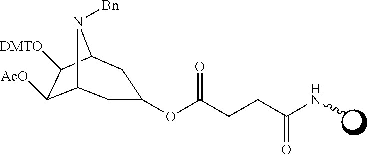 | 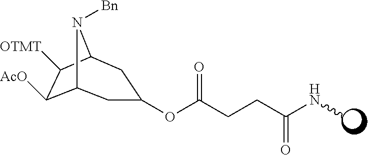 | 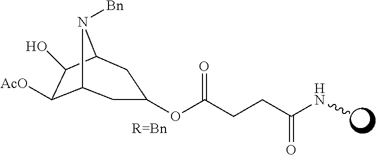 |
| R = Bn | R₃ = Me R⁴ = Bn | R = Bn |
| 734c, 734p | 834c, 834p | 934c, 934p |
| 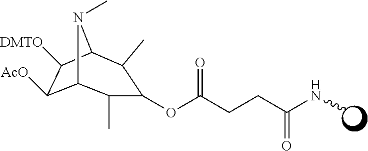 | 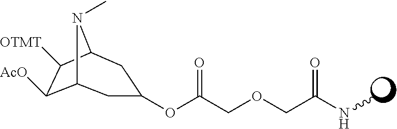 | 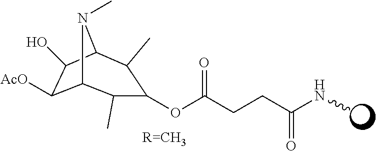 |
| R = CH₃ | R₃ = R⁴ = CH₃ | R = CH₃ |
| 735c, 735p | 835c, 835p | 935c, 935p |
| 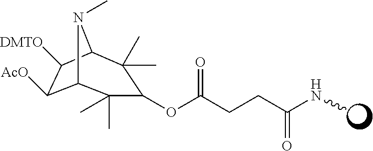 | 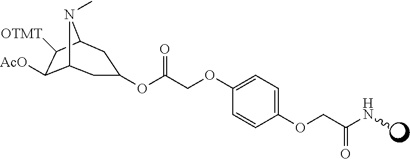 | 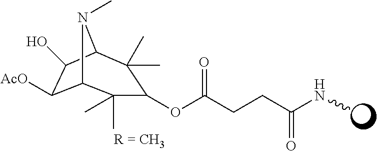 |
| R = CH₃ | R₃ = R⁴ = CH₃ | R = CH₃ |
| 736c, 736p | 836c, 836p | 936c, 936p |
| 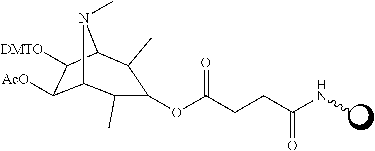 | 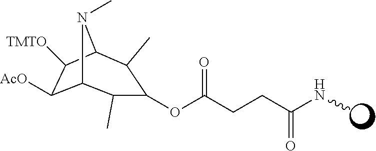 | |
| 737c, 737p | R = CH₃ | |
| | 837c, 837p | |
| 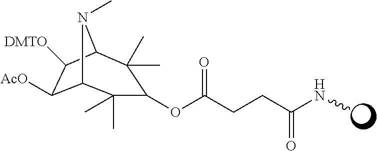 | 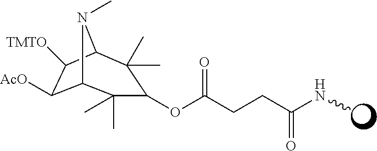 | |
| 738c, 738p | R = CH₃ | |
| | 838c, 838p | |
| 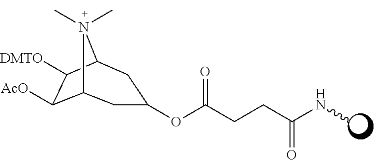 | 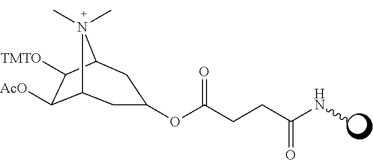 | 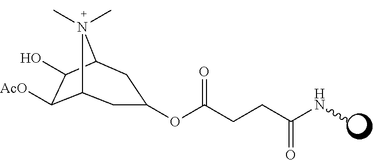 |
| R₃ = R⁴ = CH₃ | R₃ = R⁴ = CH₃ | R₃ = R⁴ = CH₃ |
| 741c, 741p | 841c, 841p | 941c, 941p |
| 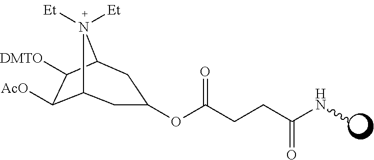 | 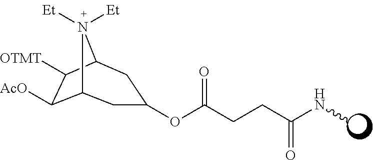 | 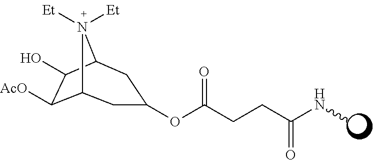 |
| R₃ = R⁴ = Et | R₃ = R⁴ = Et | R₃ = R⁴ = Et |
| 742c, 742p | 842c, 842p | 942c, 942p |
| 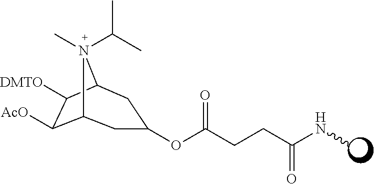 | 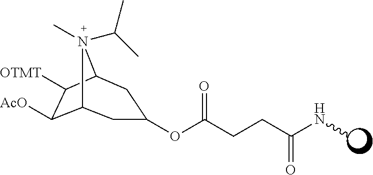 | 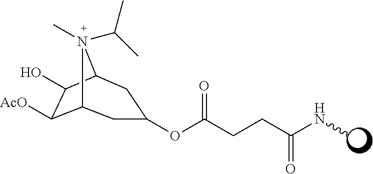 |
| R₃ = Me, R⁴ = iPr | R₃ = Me, R⁴ = iPr | R₃ = Me, R⁴ = iPr |
| 743c, 743p | 843c, 843p | 943c, 943p |
| 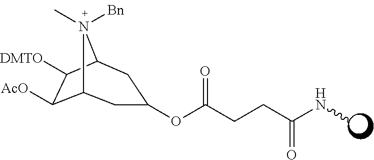 | 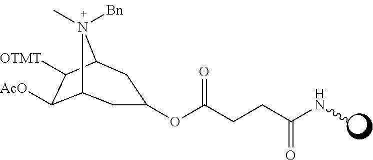 | 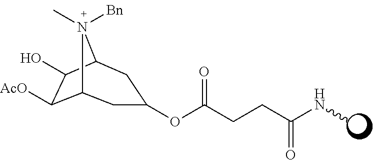 |
| R₃ = Me R⁴ = Bn | R₃ = Me R⁴ = Bn | R₃ = Me R⁴ = Bn |
| 744c, 744p | 844c, 844p | 944c, 944p |
| 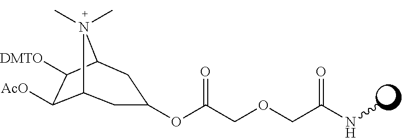 | 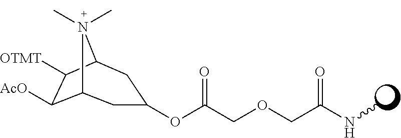 | 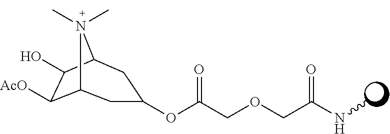 |
| R₃ = R⁴ = CH₃ | R₃ = R⁴ = CH₃ | R₃ = R⁴ = CH₃ |
| 745c, 745p | 845c, 845p | 945c, 945p |
| 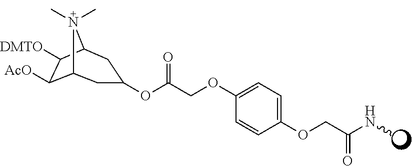 | 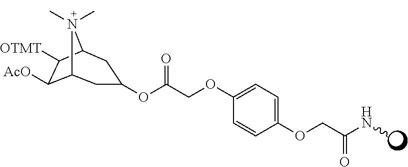 | 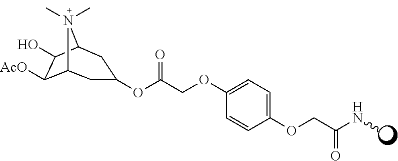 |
| R₃ = R⁴ = CH₃ | R₃ = R⁴ = CH₃ | R₃ = R⁴ = CH₃ |
| 746c, 746p | 846c, 846p | 946c, 946p |
| 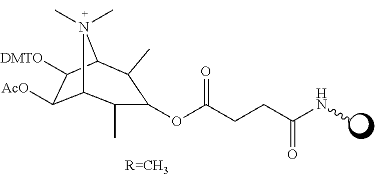 | 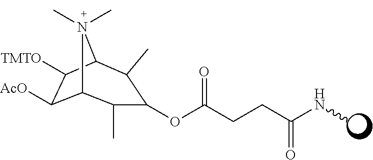 | 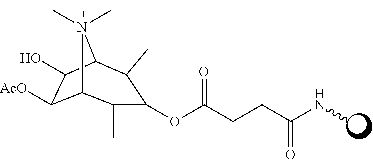 |
| R = CH₃ | R = CH₃ | R = CH₃ |
| 747c, 747p | 847c, 847p | 947c, 947p |
| 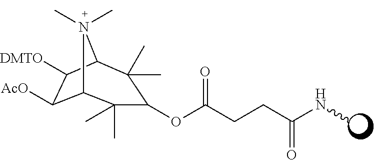 | 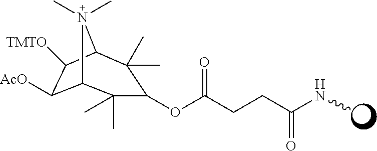 | 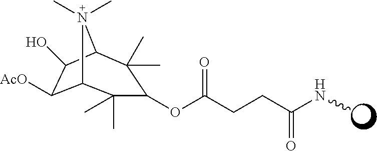 |
| R = CH₃ | R = CH₃ | R = CH₃ |
| 748c, 748p | 848c, 848p | 948c, 948p |
| 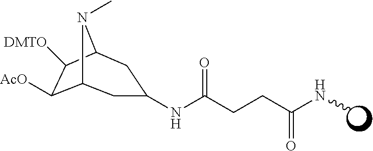 | 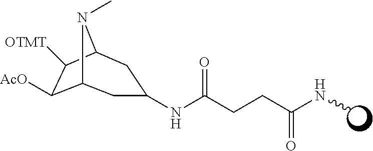 | |
| R₃ = CH₃ | R₃ = CH₃ | |
| 751c, 751p | 851c, 851p | |
| 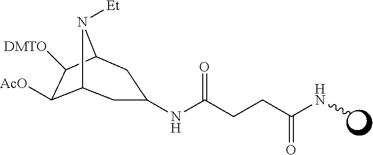 | 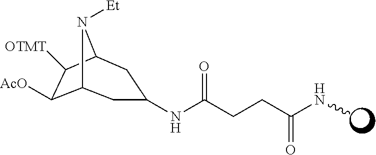 | |
| R₃ = Et | R₃ = Et | |
| 752c, 752p | 852c, 852p | |
| 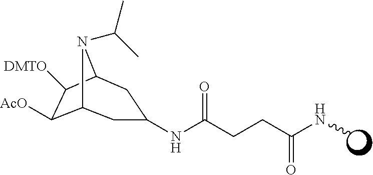 | 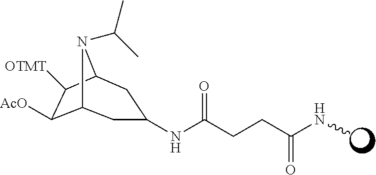 | |
| R₃ = iPr | R₃ = iPr | |
| 753c, 753p | 853c, 853p | |
| 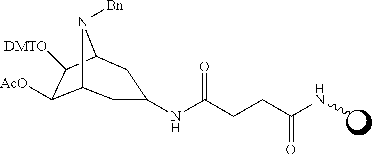 | 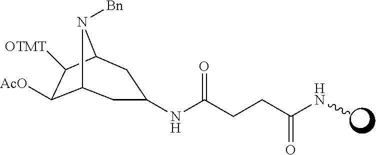 | |
| R₃ = Bn | R₃ = Bn | |
| 754c, 754p | 854c, 854p | |
| 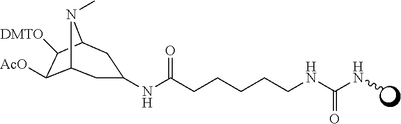 | 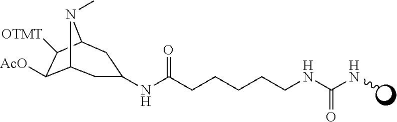 | |
| R₃ = CH₃ | R₃ = CH₃ | |
| 755c, 755p | 855c, 855p | |
| 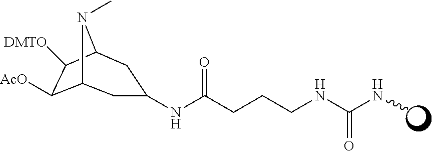 | 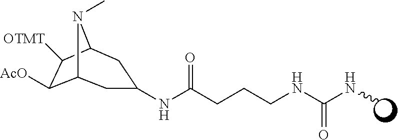 | |
| R₃ = CH₃ | R³ = CH₃ | |
| 756c, 756p | 856c, 856p | |
| 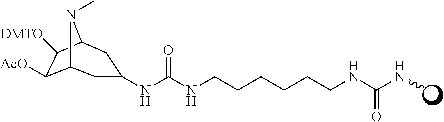 | 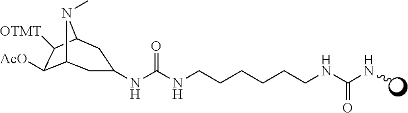 | |
| R₃ = R⁴ = CH₃ | 857c, 857p | |
| 757c, 757p | | |
| | 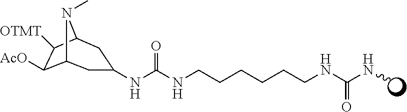 | 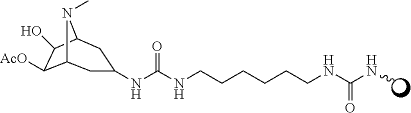 |
| | R₃ = R⁴ = CH₃ | R₃ = R⁴ = CH₃ |
| | 858c, 858p | 958c, 958p |
| | 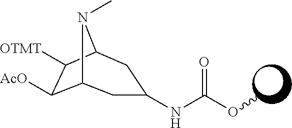 | |
| | 859c, 859p | |
| 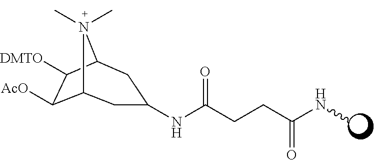 | 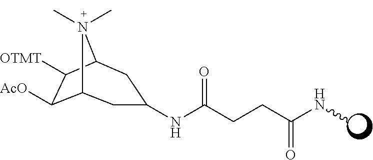 | 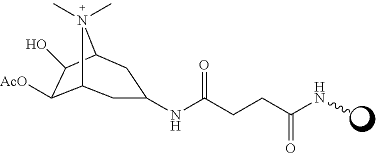 |
| R₃ = R⁴ = CH₃ | R₃ = R⁴ = CH₃ | R₃ = R⁴ = CH₃ |
| 761c, 761p | 861c, 861p | 961c, 961p |
| 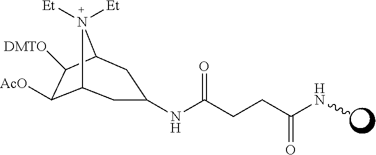 | 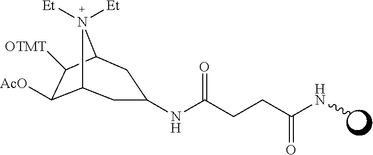 | 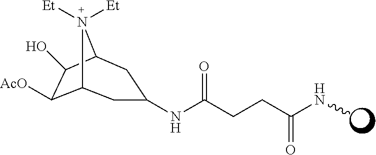 |
| R₃ = R⁴ = Et | R₃ = R⁴ = Et | R₃ = R⁴ = Et |
| 762c, 762p | 862c, 862p | 962c, 962p |
| 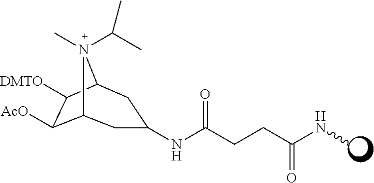 | 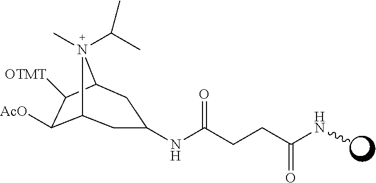 | 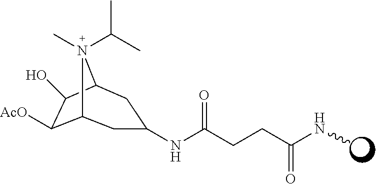 |
| R₃ = Me, R⁴ = iPr | R₃ = Me, R⁴ = iPr | R₃ = Me, R⁴ = iPr |
| 763c, 763p | 863c, 863p | 963c, 963p |
| 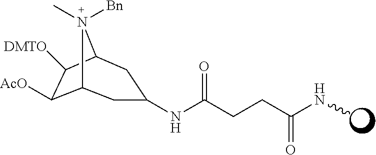 | 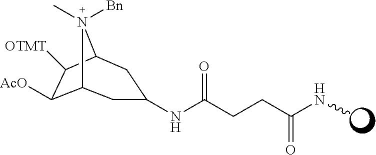 | 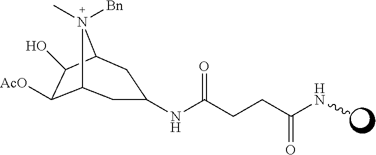 |
| R₃ = Me R⁴ = Bn | R₃ = Me R⁴ = Bn | R₃ = Me R⁴ = Bn |
| 764c, 764p | 864c, 864p | 964c, 964p |
| 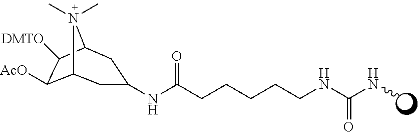 | 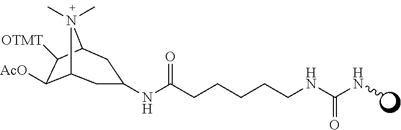 | 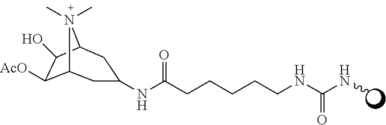 |
| R₃ = R⁴ = CH₃ | R₃ = R⁴ = CH₃ | R₃ = R⁴ = CH₃ |
| 765c, 765p | 865c, 865p | 965c, 965p |
| 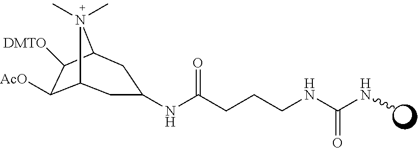 | 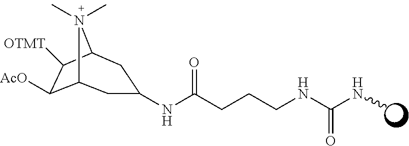 | 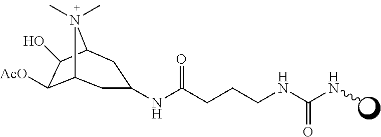 |
| R₃ = R⁴ = CH₃ | R₃ = R⁴ = CH₃ | R₃ = R⁴ = CH₃ |
| 766c, 766p | 866c, 866p | 966c, 966p |
| 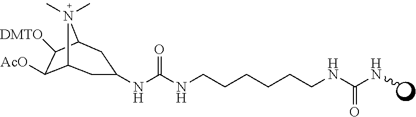 | 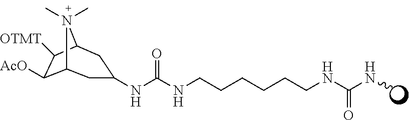 | 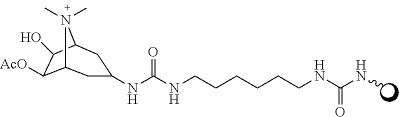 |
| R₃ = R⁴ = CH₃ | R₃ = R⁴ = CH₃ | R₃ = R⁴ = CH₃ |
| 767c, 767p | 867c, 867p | 967c, 967p |
| | 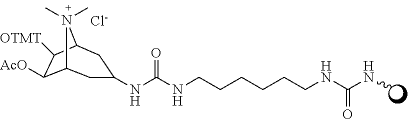 | 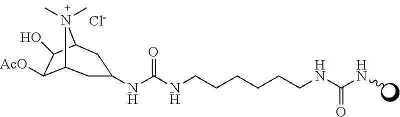 |
| | R₃ = R⁴ = CH₃ | R₃ = R⁴ = CH₃ |
| | 868c, 868p | 968c, 968p |
| | 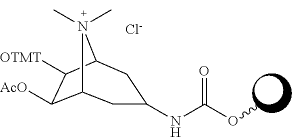 | 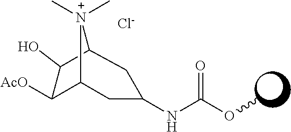 |
| | 869c, 869p | 969c, 969p |
| | 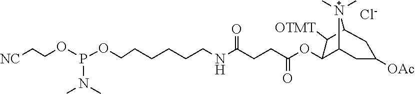 | |
| | R₃ = R⁴ = CH₃ | |
| | 871 | |
| | 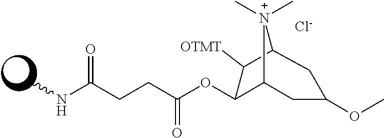 | 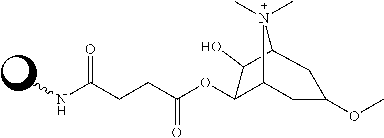 |
| | 871c, 871p | 971c, 971p |
| 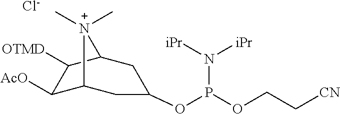 | 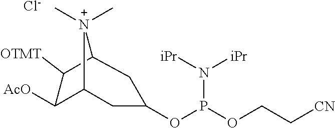 | |
| R₃ = R⁴ = CH₃ | R₃ = R⁴ = CH₃ | |
| 772 | 872 | |
| 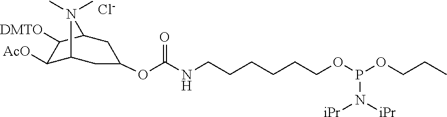 | 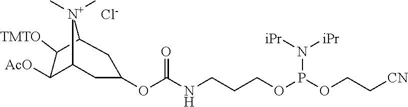 | |
| R³ = R⁴ = CH₃ | R³ = R⁴ = CH₃ | |
| 773 | 873 | |
| | 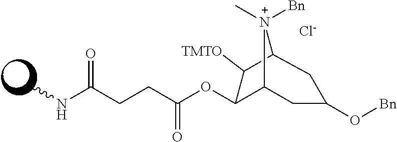 | 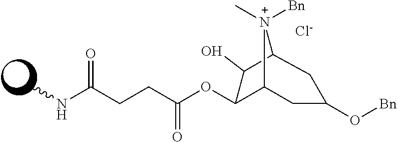 |
| | 874c, 874p | 974c, 974p |
| | 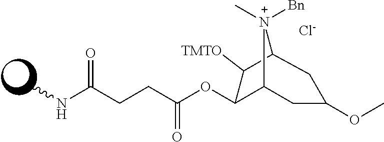 | 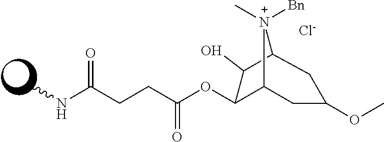 |
| | 875c, 875p | 975c, 975p |
| | 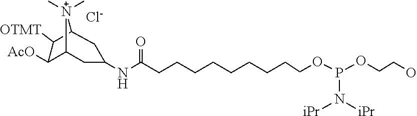 | |
| | R₃ = R⁴ = CH₃ | |
| | 874 | |
| | 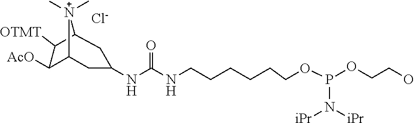 | |
| | R₃ = R⁴ = CH₃ | |
| | 875 | |
| | | 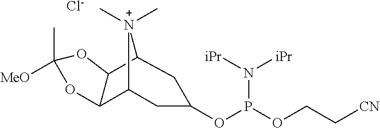 [SMILES: [Cl-] ; COC1(C)OC2C(O1)C1CC(OP(OCCC#N)N(C(C)C)C(C)C)CC2[N+]1(C)C] |
| | | R₃ = R⁴ = CH₃ |
| | | 976 |
| | | 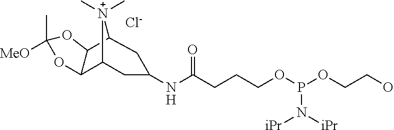 [SMILES: COC1(C)OC2C(O1)C1CC(NC(=O)CCCOP(OCCO)N(C(C)C)C(C)C)CC2[N+]1(C)C ; [Cl-]] |
| | | R₃ = R⁴ = CH₃ |
| | | 977 |
| | | 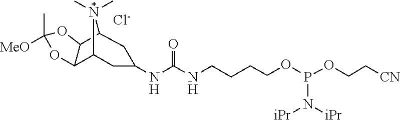 [SMILES: COC1(C)OC2C(O1)C1CC(NC(=O)NCCCCOP(OCCC#N)N(C(C)C)C(C)C)CC2[N+]1(C)C ; [Cl-]] |
| | | R₃ = R⁴ = CH₃ |
| | | 978 |

### Methods for Oligonucleotide Synthesis

Also disclosed herein is a method for functionalizing a solid phase material with a first monomeric subunit, comprising the steps of:
(a) providing a solid phase material-bound compound of Formula I

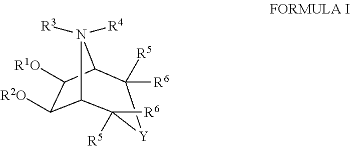

wherein:
R¹ and R² either form an orthoester function —C(CH₃)(OCH₃)— or one of R¹ and R² is hydrogen, a trityl-type protecting group, or a xanthenyl-type protecting group and the other of R¹ and R² is an acetyl, a propionyl, a n-butyryl, a benzoyl, L¹, wherein:
   L¹ is a linking moiety —C(═O)—Z—(C═O)-A¹, wherein:
      Z is selected from the group consisting of a covalent bond, a methylene group, —(CH₂)₂—, —(CH₂)—O—(CH₂)—, and —(CH₂)—O—C₆H₄—O—(CH₂)—;
      A¹ is a covalent bond to SP¹ or —NH(CH₂)ₙ—OR⁷, wherein:
         SP¹ is an oxygen, amino, an aminoalkyl or a hydroxyalkyl covalently attached to a solid phase material comprising a controlled pore glass, a magnetic controlled pore glass, a silica-containing particle, a styrene-containing polymer or copolymer, a divinylbenzene-containing polymer or copolymer, a copolymer of styrene and divinylbenzene, a controlled pore glass grafted with a styrene-containing polymer, a controlled pore glass grafted with a copolymer of styrene and divinylbenzene, a copolymer of styrene and divinylbenzene grafted with polyethylene glycol, and a flat glass surface;
         n is an integer from 2 to 10;
         R⁷ is PX, wherein:

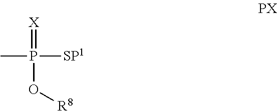

and wherein R⁸ is methyl or 2-cyanoethyl group and X is oxygen or sulfur;
R³ is selected from the group consisting of methyl, ethyl, propyl, isopropyl, and benzyl;
R⁴ is selected from the group consisting of a lone electron pair, hydrogen, methyl, ethyl, propyl, isopropyl, and benzyl, wherein when R⁴ is other than a lone electron pair, N has a positive charge and forms a salt with a halide anion or with an intramolecular carboxylic functional group;
R⁵ and R⁶ are independently hydrogen or methyl;
Y is —(C═O)—, —CH(OR⁹)—, —CH(NR¹⁰R¹¹)—, or —[C(OR¹²)(OR¹³)]—, wherein:
   R⁹ is hydrogen, methyl, ethyl, benzyl, acetyl, propionyl, butyryl, isobutyryl, pivaloyl, benzoyl, t-butyldimethylsilyl, triisopropylsilyl, or (t-butyl) diphenylsilyl group, L¹, PX, or —(CH₂)ₘ—O-PX, wherein:
      m is an integer ranging from 2 to 10;
   R¹⁰ is L¹, —(C═O)-A¹, or —(C═O)-W¹-(CH₂)ₚ—W², wherein:
      W¹ is —(CH₂)—, —(NH)—, or —(NH)—(C═O)—;
      W² is —O—PX, or —[NH(C═O)]-A¹; and
      p is an integer ranging from 2 to 10;
   R¹¹ is hydrogen, methyl, ethyl, or benzyl; and
   R¹² and R₁₃ together form ketal bridges —(CH₂)₂— or —CH₂—[C(CH₃)₂]—CH₂—;
wherein only one of R¹, R², and Y may be or include L¹ or PX;
(b) selectively removing one of the protecting groups of Formula I to form a reactive hydroxyl group;
(c) providing a first monomeric subunit comprising an activated phosphorus group and a protected hydroxy group, and reacting the activated phosphorus group of the first monomeric subunit with the reactive hydroxyl group of the compound of Formula I to form a monomer-functionalized solid support comprising a phosphite group;
(d) treating said monomer-functionalized solid support with a capping agent and/or treating said monomer-functionalized solid support with an oxidizing solution or a sulfurizing agent to convert the phosphite triester group to a phosphotriester or phosphothioate triester, thereby forming an oxidized or sulfurized functionalized solid support;
(e) optionally repeating steps (b), (c), and (d) one or more times for the oxidized or sulfurized functionalized solid support to form an oligomeric-functionalized solid support, wherein the monomeric subunit is the same or different each time steps (b), (c), and (d) are repeated.

In some embodiments, the method further comprising deprotecting the oligomeric-functionalized solid support of step (e) and cleaving the oligomeric-functionalized solid support to form an oligomeric compound separate from the solid phase material, wherein the cleaving forms a terminal hydroxy group on the oligomeric compound at the site of cleavage.

In some embodiments of the method, one of R¹ or R² is L¹.

In some embodiments of the method, R⁷ is PX.

In some embodiments of the method, the activated phosphorus group comprises a phosphoramidite, an H-phosphonate, or a phosphate triester.

In some embodiments of the method, the oligomeric compound is an oligonucleotide, optionally comprising unnatural sugar-modified nucleotide residues, unnatural base-modified nucleotide residues, or non-nucleotide monomeric units.

The solid-phase-material-bound compound of Formula I can be any of the compounds described herein that are bound to a solid phase material. For example, the solid-phase-material-bound compound of Formula I can be any compound listed in Table 1 or throughout the Examples. For example, the solid-phase material-bound compound of Formula I can be 701-707c/p, 711-713c/p, 721-728c/p, 731-738c/p, 741-748c/p, 751-757c/p, or 761-767c/p. For example, the solid-phase material-bound compound of Formula I can be 801-807c/p, 811-819c/p, 821-828c/p, 831-838c/p, 841-848c/p, 851-759c/p, 861-869c/p, 871c/p, 874c/p, or 875c/p. For example, the solid-phase material-bound compound of Formula I can be 901-905c/p, 911-919c/p, 925-928c/p, 931-936c/p, 941-948c/p, 958c/p, 961-969c/p, 971c/p, 974c/p, or 975c/p.

The step of deprotecting a hydroxyl group of Formula I by selectively removing one of the protecting groups can be accomplished by any method known to one skilled in the art depending on which protecting group is to be selectively removed. For example, a trityl-type protecting group can be removed via treatment with 75%, 76%, 77%, 78%, 79%, 80%, 81%, 82%, 83%, 84%, 85%, 86%, 87%, 88%, 89%, 90%, 91%, 92%, 93%, 94%, or 95% trifluoroacetic acid. Alternatively, the trityl-type protecting groups can be removed with 5% anhydrous solution of tricholoro- or trifluoroacetic acid in toluene to yield unprotected solid supports. For example, a xanthenyl-type protecting group can be removed via treatment with 75%, 76%, 77%, 78%, 79%, 80%, 81%, 82%, 83%, 84%, 85%, 86%, 87%, 88%, 89%, 90%, 91%, 92%, 93%, 94%, or 95% trifluoroacetic acid. Additional methods of selectively removing protecting groups are described in the disclosed examples and are known to those skilled in the art.

Each monomeric subunit comprising an activated phosphorus group and a protected hydroxy group may be reacted with the compound of Formula I by methods known in the art, including but not limited to methods described in the specific examples disclosed herein. In some examples, each monomeric subunit is a nucleotide or derivative thereof. The reactive phosphorus group on the monomeric subunit can include, but is not limited to a phosphoramidite, an H-phosphonate, or a phosphate triester. The protected hydroxy group can include any protecting group known in the art. In some examples, the protected hydroxy group includes a trityl-type protecting group, such as but not limited to 4-methoxytrityl, 4,4′-dimethoxytrityl, or 4,4′,4″-trimethoxytrityl. In other examples, the protected hydroxy group includes a xanthenyl-type protecting group, such as but not limited to 9-phenylxanthen-9-yl or 9-(4-methoxyphenyl)xanthen-9-yl. A monomeric subunit comprising an activated phosphorus group and a protected hydroxy group is reacted with the reactive hydroxyl group of the compound of Formula I to form a monomer-functionalized solid support comprising a phosphite group.

The step of treating the monomer-functionalized solid support with a capping agent can be accomplished by methods known in the art, including but not limited to methods described in the specific examples disclosed herein. In some examples, the capping agent is an organic ligand, organic amine, a thiol, or a pyridine based capping agent. Treating with the capping agent produces a 5′-capped oligonucleotide bound to a universal solid support. The step of treating the monomer-functionalized solid support with an oxidizing solution or a sulfurizing agent can be accomplished by methods known in the art, including but not limited to methods described in the specific examples disclosed herein. In some examples, the oxidizing solution is composed of THF: pyridine: water. Treating with the oxidizing solution converts the phosphite triester group to a phosphotriester. In some examples, the sulfurizing agent is DDTT (3-[[(dimethylamino)methylene]amino]-3H-1,2,4-dithiazole-5-thione). Treating with the sulfurizing agent converts the phosphite triester group to a phosphothioate triester.

Optionally, steps (b), (c), and (c) can be repeated to add multiple monomeric subunits to form an oligomeric functionalized solid support. When multiple monomeric subunits are reacted to form an oligomer, the monomeric units can be the same or different, depending on the structure of the desired oligomer.

When the method further includes deprotecting the oligomeric functionalized solid support of step (e) and cleaving the oligomeric-functionalized solid support to form an oligomeric compound separate from the solid phase material, the cleaving forms a terminal hydroxy group on the oligomeric compound at the site of the cleavage. The step of deprotecting the oligomeric functionalized solid support can be accomplished by any method known to one skilled in the art depending on which protecting group is to be selectively removed. For example, certain methods of deprotecting the oligomeric functionalized solid support are described in the examples disclosed herein. The step of cleaving to form an oligomeric compound separate from the solid phase material also can be accomplished by any method known to one skilled in the art, including but not limited to methods described in the specific examples disclosed herein.

The examples below disclose methods of synthesizing certain compounds of Formula (I). The skilled artisan will appreciate that those compounds and others falling within the general scope of Formula I could be made by methods other than those specifically described herein, by adaptation of the methods described herein, and/or by adaptation of methods known in the art. In general, compounds provided herein may be prepared in a multi-step synthesis, as shown below. All quantities shown are approximate, and are given solely for illustrative purposes.

### EXAMPLES

The following examples are intended to further illustrate certain preferred embodiments of the invention and are not intended to be limiting in nature.

### Example 1: Preparation of the preferred linkers for universal solid supports

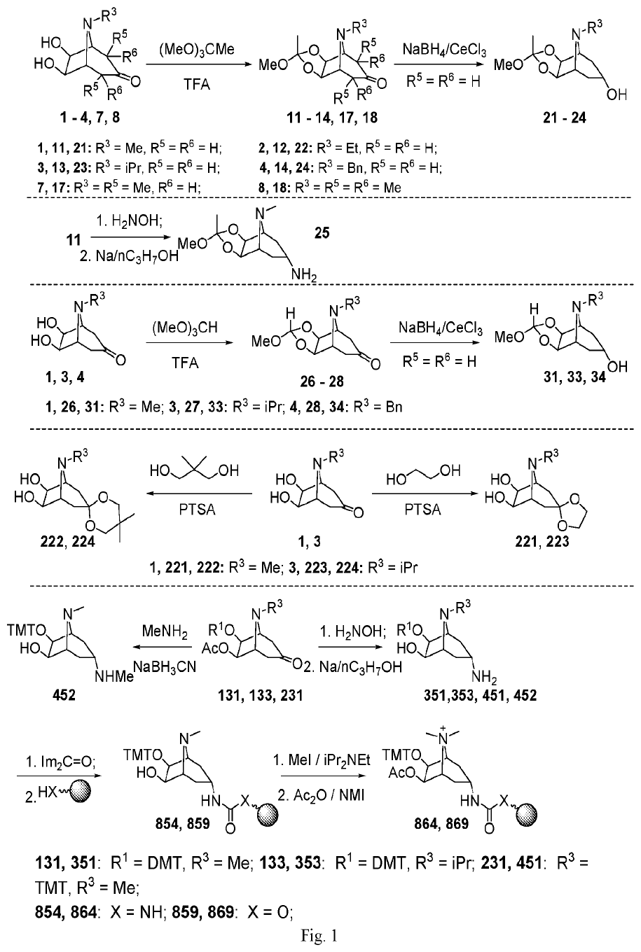

### Synthesis of Starting Materials (FIG. 1)

Compounds 1-3 were prepared as disclosed in Villacampa, M; Martinez, M.; Gonzalez-Trigo, G.; Sollhuber, M. M. Synthesis and stereochemistry of (3a)-6B, 7β-dihydroxy- and 6β-hydroxy-8-alkyl-8-azabicyclo[3.2.1]octane-3-spiro-5′-imidazoline-2′,4′-diones. J. Heterocycl. Chem. 1992, 29 (6), 1541-4. Compound 4 was prepared as disclosed in Buchi, G.; Fliri, H.; Shapiro, R. Synthesis of betalains. J. Org. Chem. 1978, 43, 25, 4765-9. Compounds 7 and 8 were prepared as disclosed in Riche, F.; Masri, F.; Lopez, M. Diastereoselective synthesis of polyfunctionalized piperidines as precursors of dopamine transporter imaging agents. Tetrahedron Lett. 2007, 48, 9, 1609-1612, Paparin, J.-L.; Crevisy, C.; Toupet, L.; Gree, R. Synthesis and functionalization of new tropanes designed for use as scaffolds in combinatorial chemistry. Eur. J Org. Chem. 2000, (23), 3909-3918, and in U.S. Pat. No. 4,001,249, respectively.

Conversion of 1-4, 7, and 8 to orthoesters 11-14, 17, 18, and 26-28 was carried out as disclosed in Buchi, G.; Fliri, H.; Shapiro, R. Synthesis of betalains. J. Org. Chem. 1978, 43, 25, 4765-9.

Compounds 216, 218, and 221-224 were prepared as disclosed in Affolter, O; Baro, A.; Laschat, S.; Fischer, P. Acylation of tropane alkaloids displaying reversed diastereoselectivities under enzymatic versus chemical conditions. Z. Naturforsch., B: Chemical Sciences 2007, 62 (1), 82-92.

Compounds 25, 351,353, and 451 were prepared from 11, 131, 133, and 231, respectively, by action of hydroxylamine hydrochloride in pyridine followed by reduction with Li AlH₄ as disclosed in Lewin, A. H.; Sun, G.; Fudala, L.; Navarro, H.; Zhou, L.-M.; Popick, P.; Faynsteyn, A.; Skolnick, P. Molecular Features Associated with Polyamine Modulation of NMDA Receptors. J. Medicinal Chem. 1998, 41 (6), 988-995.

Compound 452 was prepared by reductive amination were prepared as disclosed in WO2009109477 and, alternatively, as in Baxter, Ellen W.; Reitz, Allen B. Reductive aminations of carbonyl compounds with borohydride and borane reducing agents. Organic Reactions (Hoboken, NJ, United States) 2002, p. 59.

### Synthesis of Unprotected Universal Solid Supports 911, 912, and 913

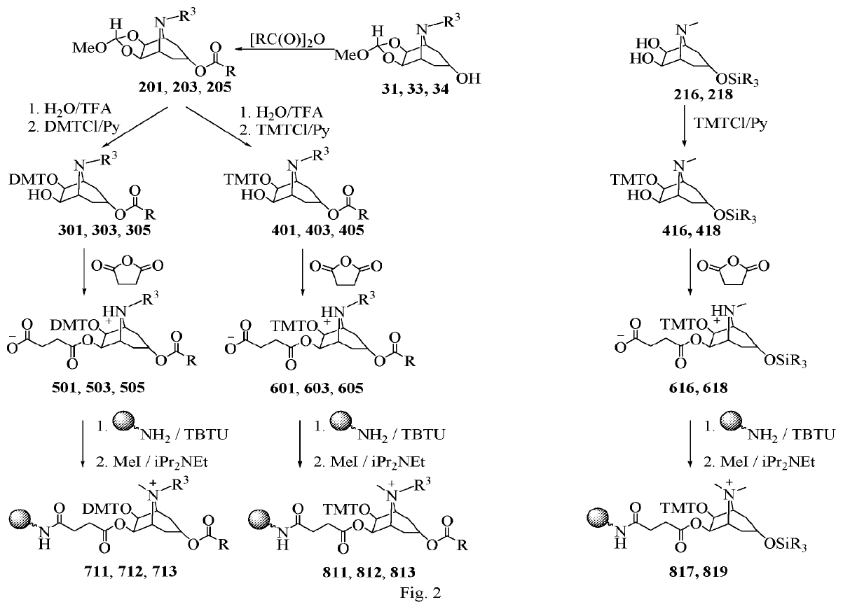

In a preferred embodiment, compounds 31, 33, and 34 were acylated at the free hydroxy group under the standard conditions by the respective carboxylic acid anhydrides in pyridine as shown in FIG. 2. The products, orthoesters 201, 203, and 205 were subjected to mild acidic hydrolysis using catalytic TFA and a 3-fold excess water. Under the optimized conditions disclosed in Example 35, the hydrolysis led to the selective formation of the respective monoformates, which, upon removal of excess water by filtering through the drying agent, was subjected to the treatment with DMT- or TMT-chlorides followed by the hydrolytic removal of the formate protecting group catalyzed by excess TEA to give the DMT-protected compounds 301, 303, and 305 and their TMT-protected counterparts 401, 403, and 405. The latter were succinylated by a 3-fold excess succinic anhydride under the standard conditions. The obtained hemisuccinates 501, 503, and 505 and 601, 603, and 605 were attached to aminopropyl CPG of various pore sizes and to aminomethyl MPPS. The loading of these USS and all other USS of the present application was in agreement with the pore size and the specific surface area of the respective solid support material (30-35, 40-50, 70-80, and 320-340 μmol/g for CPG1500, CPG1000, CPG500, and MPPS, respectively). Finally, the bridging tertiary amino group was quaternized using excess methyl iodide and DIPEA to give novel DMT- and TMT-protected versions of universal solid supports 711, 712, and 713 and 811, 812, and 813, respectively.

Solid supports protected with a trityl-type protecting groups are routinely used in oligonucleotide synthesis wherein said protecting group is removed under the acidic conditions at the beginning of the synthesis. However, it more convenient for the end user that the detritylated universal solid support are installed on the instrument thereby allowing said end-user to skip the initial detritylation subroutine, with the associate economy of time, reagents, and solvents. Accordingly, universal solid supports 811, 812, and 813 were detritylated with 5% anhydrous solution of trichloro- or trifluoroacetic acid in toluene to give the unprotected universal solid supports 911, 912, and 913.

### Synthesis of Universal Solid Supports 817 and 819

In another preferred embodiment, 3-O-silyl protected triols 216 and 218 were protected at one of the available hydroxy groups by reacting with TMT chloride (FIG. 2). The steric bulk of the TMT group introduced first retards the reactivity of the other hydroxy group substantially. Accordingly, the mono-TMT-protected species 416 and 418 were obtained in 69-72% yields and were isolated by column chromatography on silica gel. The following succinilation afforded hemisuccinate 616 and 618 whose attachment to aminopropyl CPG and to aminomethyl MPPS followed by quaternization with methyl iodide gave the 3′-O-sily protected USS 817 and 819. These were detritylated with anhydrous solution of trichloro- or trifluoroacetic acid in toluene under the standard conditions to give universal solid supports 817 and 819.

### Synthesis of Unprotected Universal Solid Supports 971, 974, and 975

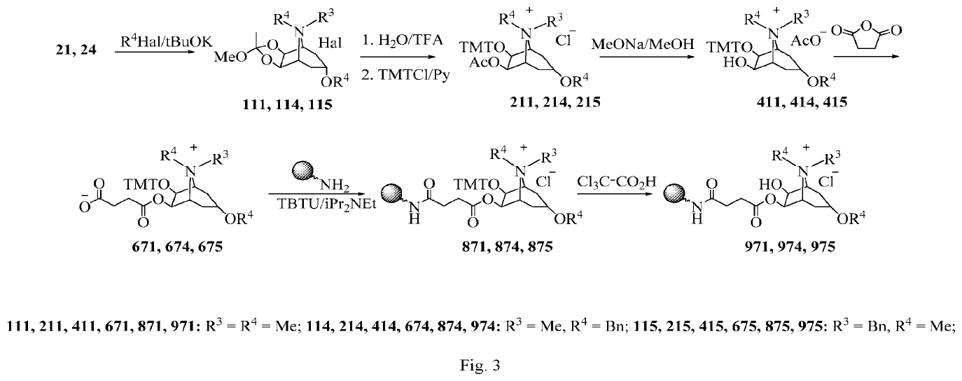

In another preferred embodiment, as illustrated in FIG. 3, orthoacetate-protected compounds 21 and 24 were alkylated with methyl iodide or benzyl bromide simultaneously at the amino and the 3-hydroxy positions to give quaternary salts 111, 114, and 115. Upon the acidic hydrolysis of the orthoacetate protection, the monoacetates were obtained and, without purification, were protected at the 7-hydroxy position with the TMT group to give 211, 214, and 215. In the following step, the removal of the acetyl group by methanolic sodium methoxide led to 411, 414, and 415 purified by column chromatography on silica gel. Following the standard procedures described above, 411, 414, and 415 were reacted with succinic anhydride. The hemisuccinates 671, 674, and 675 thus obtained were attached to aminopropyl CPG and aminomethyl MPPS to yield the TMT-protected USS 871, 874, and 875. Finally, the TMT protection was removed by treating with 5% trichloroacetic acid in toluene to give the unprotected universal solid supports 971, 974, and 975.

### Synthesis of Universal Solid Supports 921-924

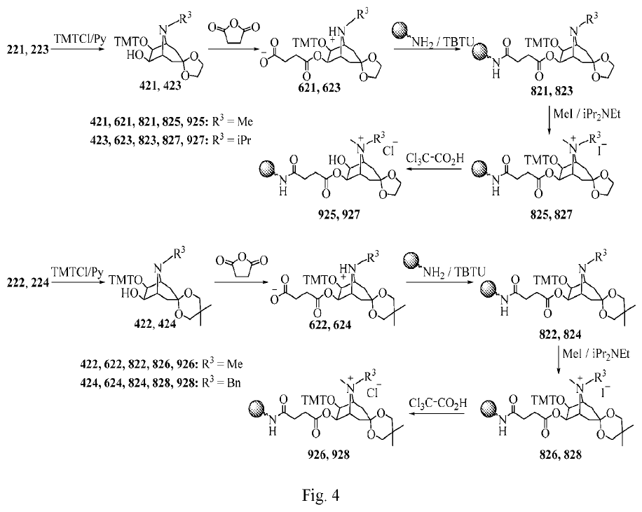

In yet another preferred embodiment, readily available [3, 2′]spirodioxolane and [3, 2′]spirodioxane compounds 221-224 featuring a ketal protection of the carbonyl group in 6,7-dihydroxy-8-alkyltropan-3-one were mono-protected with the TMT group at the 7-O-position (FIG. 4). In the next step, thus obtained 421-424 were succinylated at the 6-O-position to give compounds 621-624. Attachment of the hemisuccinates 621-624 to aminopropyl CPG and to aminomethyl MPPS gave 821-824. The latter were quaternized to 821-824, which were detritylated to give [3, 2′]spirodioxolane and [3, 2′]spirodioxane universal solid supports 921-924. The spiroketal protection in all four supports was found stable towards the acidic conditions of anhydrous detritylation.

### Using 6-O-DMT- and 6-O-TMT-3-Amino Derivatives 351, 353, 451, and 452 of FIG. 1

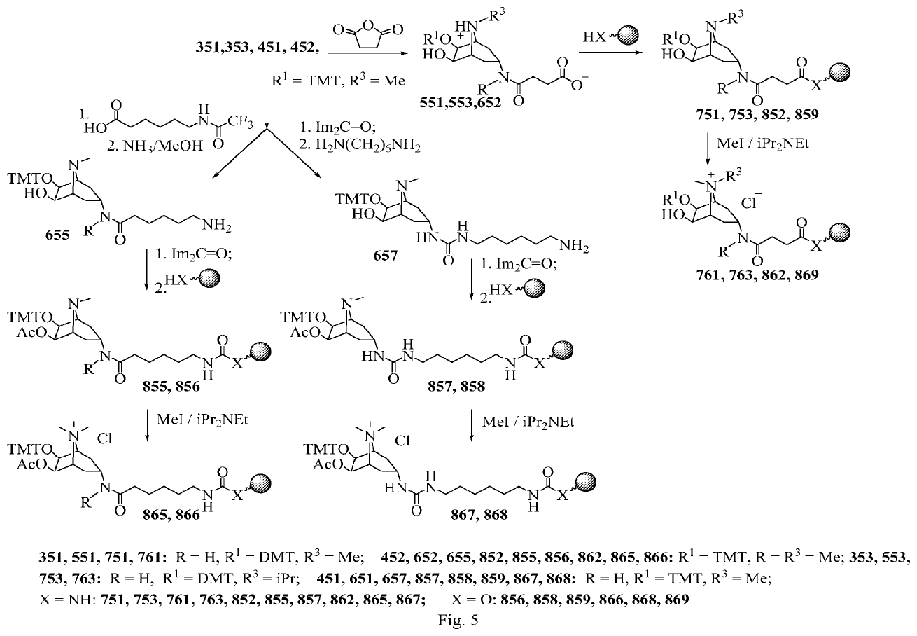

In one implementation of this preferred embodiment, compounds 351, 353, and 452 were selective succinylated to form internal salts of succinamic acid 551, 553, and 652 (FIG. 5). There were attached to aminopropyl- and hydroxypropyl CPG and aminomethyl and hydroxymethyl MPPS to give solid supports 751, 753, 852, and 859. The latter were converted to DMT-protected quaternary ammonium universal solid supports 761 and 763 and to the TMT-protected 862 and 869. Finally, 862 and 869 were detritylated to 962 and 969.

In another implementation of the preferred embodiment, a side chain was attached to the amino function of 451 by activation of said amino function with carbonyldiimidazole followed by the reaction with 1,6-diaminohexane to form compound 657. Upon column purification, 657 was activated with carbonyldiimidazole, and the activated species was loaded on both amino- and hydroxy-functionalized CPG and MPPS to form urea- and carbamate-immobilized universal solid supports 857 and 858, respectively. 8-N-Methylation conducted as disclosed above gave 867 and 868, and the following detritylation led to the unprotected universal solid supports 967 and 968.

In yet another implementation of the preferred embodiment, the TMT-protected compound 452 was acylated at the amino group with an N-oxysuccinimidyl ester of 6-(trifluoroacetamido)hexanoic acid, which was followed by the removal of the trifluoroacetyl protecting group by methanolic ammonia. Upon column purification, the product 655 was activated with carbonyldiimidazole as disclosed in the previous paragraph, and the activated species was loaded on both amino- and hydroxy-functionalized CPG and MPPS to form urea- and carbamate-immobilized universal solid supports 855 and 856, respectively. 8-N-Methylation of these gave, respectively, universal solid supports 865 and 866, which were detritylated to 965 and 966.

Except for 969, all linkers in universal solid supports of this embodiment are attached to the solid phase material via chemically stable spacers which are resistant towards deprotection conditions employing ammonia or methylamine. Accordingly, only 3′-dephosphorylated oligonucleotide products are expected to be released to the solution during deprotection.

### Synthesis of Universal Solid Supports 941-944, 947, and 948

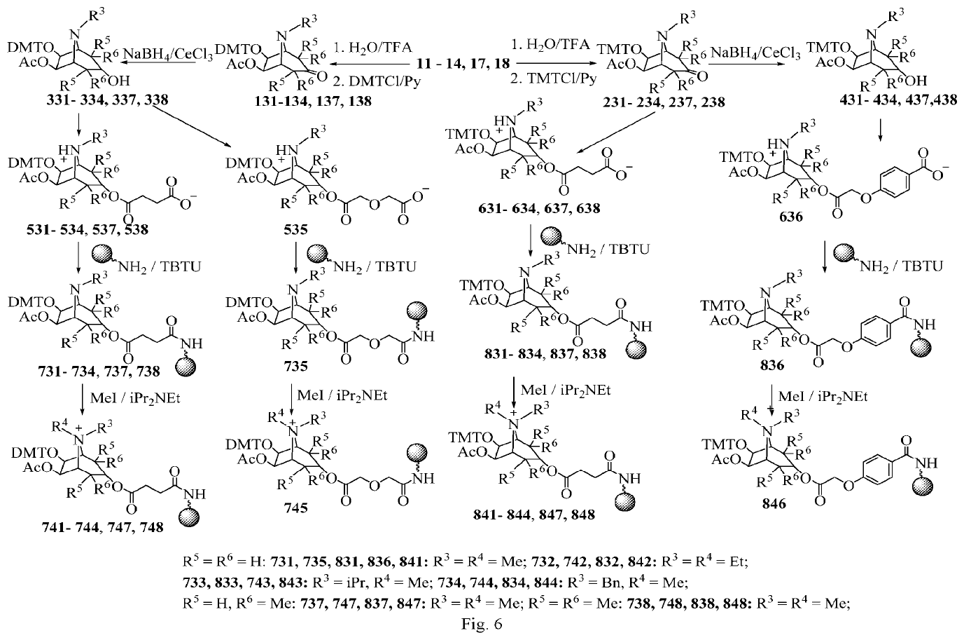

In the most preferred embodiment, the 3-O-position of the tropane system is used for the attachment of universal linker to solid phases as shown in FIG. 6. Compounds 11-14, 17, and 18 protected at their vicinal hydroxy groups with orthoacetate moiety are the most readily synthetically available among all starting materials in this application. Upon mild acid-catalysed hydrolysis of the orthoacetate protection with aqueous trifluoroacetic acid (TFA), the released hydroxy group was protected by reaction with DMT chloride to give compounds 131-134, 137, and 138 or with TMT chloride to obtain 231-234, 237, and 238. The carbonyl function in all tested compounds was resistant towards conventional reduction with NaBH₄, but addition of catalytic CeCl₃ led to the smooth reductions to give alcohols 331-334, 337, and 338 and 431-434, 437, and 438 without any loss of the acetyl group. In the next step, the standard succinylation produced 531-534, 537, and 538 and 631-634, 637, and 638. Similarly, treatment of 331 with diglycolic anhydride gave 535. Compound 636 was obtained from 431 and 1,4-hydroquinone diacetic acid activated with disuccinimidyl carbonate. All linkers obtained were attached to aminopropyl CPG and aminomethyl MPPS by following the standard protocol. The loading of all universal solid supports was commensurate with the pore size and the specific surface area of the respective solid support materials. The following methylation with iodomethane/DIPEA led to DMT-protected USS 741-744, 747, and 748, and to TMT-protected universal solid supports 841-844, 847, and 848. The TMT-protected universal solid supports were further detritylated to 941-944, 947, and 948.

### Example 2: Providing Universal Phosphoramidites for Oligonucleotide Synthesis

Such phosphoramidites are coupled to underivatized solid support wherein aminoalkyl- or hydroxyalkyl groups are present and thus convert said supports to universal solid supports. This technique is of advantageous for oligonucleotide synthesis on miniature scale including but not limited to generation of oligonucleotide microarrays on glass slides or other carriers.

### Synthesis of Universal Phosphoramidite 806a

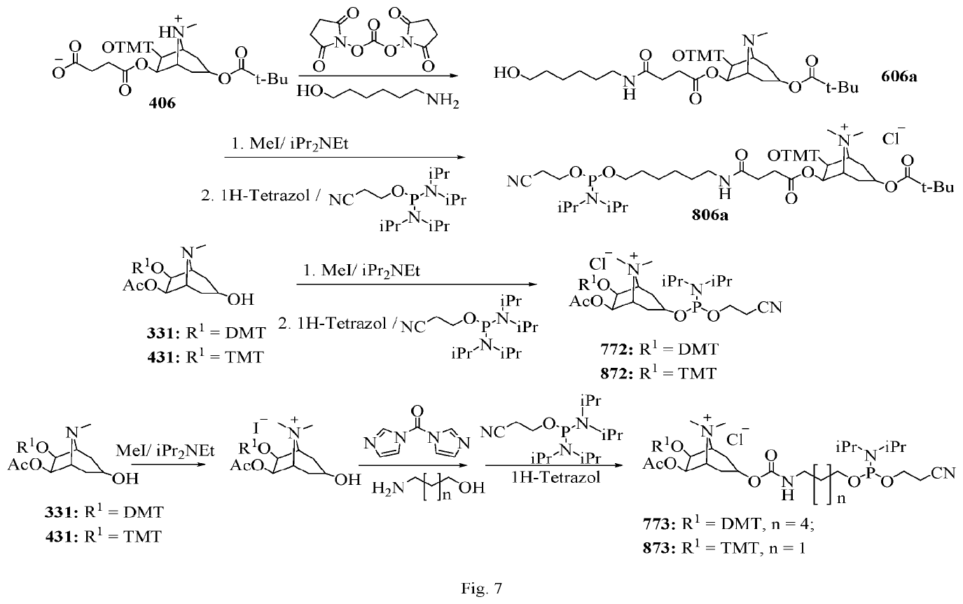

In one preferred embodiment, hemisuccinate 406 was activated with N,N-disuccinimidyl carbonate to form a reactive N-oxysuccinimidy ester, which was subsequently treated with 6-aminohexanol to give compound 606a (FIG. 7). This was methylated at the 8-amino position with iodomethane in the presence of DIPEA and finally converted to universal phosphoramidite 806a in the standard manner using 2-cyanoethyl bis(N,N-diisopripylamino) phosphite activated by 1H-tetrazol.

### Synthesis of Universal Phosphoramidites 772-773 and 872-873

In another embodiment, compounds 331 and 431 disclosed above were methylated by using iodomethane/DIPEA (FIG. 7). The products were converted to DMT- and TMT-protected universal phosphoramidites 772 and 872.

Alternatively, 331 and 431 were methylated as disclosed above. In the next step, a side chain was connected to the 3-O-position by activating the 3-hydroxy function with 1,1-carbonyldiimidazole followed the formation of carbamate derivatives by reaction either with 3-aminopropanol or with 6-aminohexanol. Upon column purification, these were converted to the respective phosphoramidites 773 (R¹=DMT, n=4) and 873 (R¹-TMT, n=1) as shown in FIG. 7.

### Synthesis of Universal Phosphoramidite 877

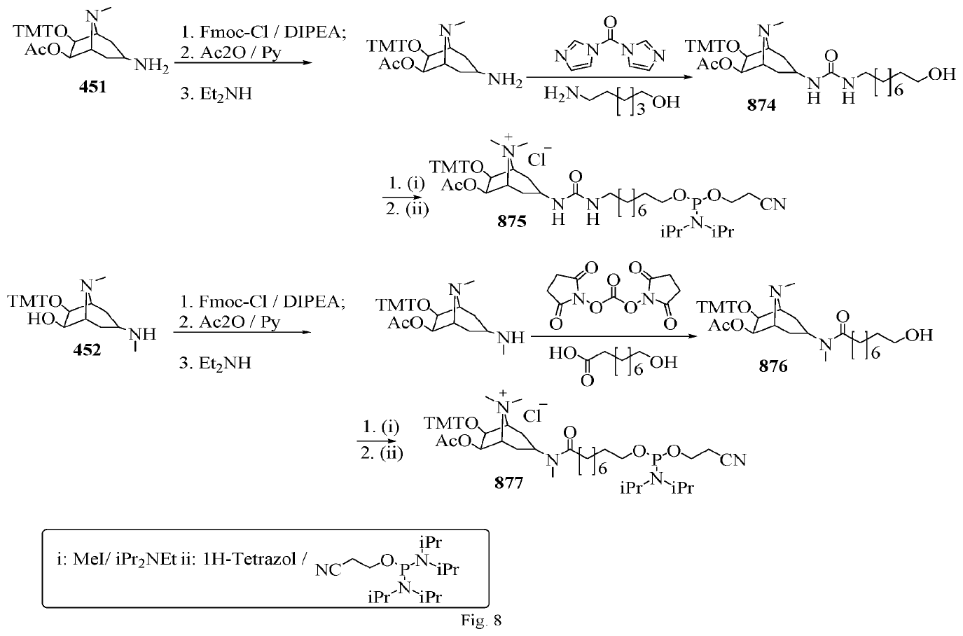

In yet another embodiment, compound 451 was first protected at its primary amino function with Fmoc-Cl in the presence of DIPEA (FIG. 8). Without further purification, the product was acetylated at the 6-O-position with acetic anhydride in pyridine, and Fmoc protecting group was selectively removed by treatment with diethylamine. The product was reacted with 1,1-carbonyldiimidazole followed by the addition of 6-aminohexanol to form the urea derivative 874. This was methylated with iodomethane in the presence of DIPEA, and the quaternary ammonium salt formed was converted to the phosphoramidite 875 by the standard protocol of this reaction. In a similar manner, compound 452 was acetylated at the 6-O-position by the temporary protection of the secondary amino function with Fmoc, after which the Fmoc protection was removed to expose said amino function. In the next step, said secondary amino function was acylated by 9-hydroxynonanoic acid pre-activated to its N-oxysuccinimidyl ester N, N-disuccinimidyl carbonate to give the extended alcohol 876. The two remaining steps of methylation and conversion to the phosphoramidite were conducted in the standard manner to yield compound 877.

### Synthesis of Universal Phosphoramidites 881 and 883

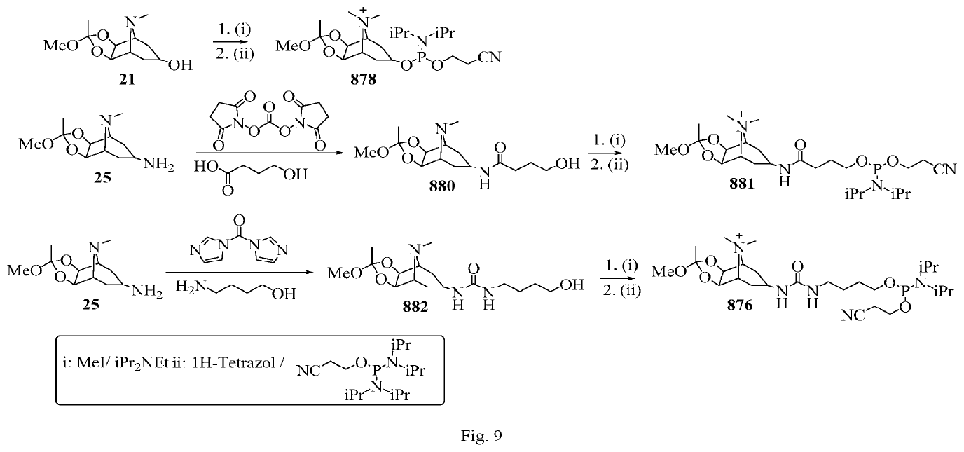

In the most preferred embodiment, the universal phosphoramidites wherein the vicinal hydroxy groups are protected with the orthoacetate function were generated (FIG. 9). Accordingly, readily available compound 21 was methylated as described above and was converted to the phosphoramidite 278 in a very simple and expedite procedure. Skilled artisans will appreciate the fact that in certain cases a distance between the surface of the solid support and the linker is required by a specific application. This requirement is fulfilled by generation of phosphoramidites 881 and 883. Thus, compound 25 was acylated N-oxysuccinimidyl 6-hydroxybutyrate to give the extended compound 880, which, after methylation and reaction with 2-cyanoethyl bis(N,N-diisopropylamino) phosphite in the presence of 1H-tetrazole gave universal phosphoramidite 881. The phosphoramidite 883 was synthesized in the same manner in three steps starting from the orthoester-protected amine 25 except for the extension arm was introduced via a urea function as shown in FIG. 9.

### Example 3: Methods for Deprotection of Oligonucleotides Assembled on Universal Solid Supports with Simultaneous 3′-Dephosphorylation of Said Oligonucleotides

Here, several 2-deoxyoligonucleotides, their 2′-O-Me analogs and their phosphorothioate analogs were synthesized on a number of universal solid supports of the present disclosure, among which the properties of universal solid support 941c was studied in most detail.

### Tested Activators

Three different activators were tested and were found to be equally effective with the standard coupling time of 20 s on 1 μmol scale:
1) 1 M 4,5-dicyanoimidazole+0.1 M N-methylimidazole in acetonitrile;
2) 0.1 M 5-[3, 5-bis(trifluoromethyl)phenyl]-2H-tetrazole in acetonitrile (Activator 42®), and
3) 0.45 M 5-(benzylthio)-1H-tetrazole in acetonitrile.
   Oxidation solutions with different compositions of I₂, pyridine and water were tested:
1) 0.02 M I₂ in 89:10:1 THF:Water:Pyridine;
2) 0.02 M I₂ in 88:10:2 THF:Pyridine:Water, and
3) 0.05 M I₂ in 88:10:2 THF:Pyridine:Water.

The oxidation solution of composition having 0.05 M 12 in 88:10:2 THF:Pyridine:Water, was determined to be most efficient.

### Sulfurization Reaction

The sulfurization reaction was performed with 0.1 M DDTT (3-[[(dimethylamino)methylene]amino]-3H-1,2,4-dithiazole-5-thione) in anhydrous pyridine for a period of 10 minutes and was conducted prior to the capping step.

### Deprotection and Cleavage of Oligonucleotides

Postsynthetically, all oligonucleotides were treated with 30% aq. diethylamine: acetonitrile (1:4) to remove the cyanoethyl protecting groups.

Cleavage of oligonucleotides from solid supports and the removal of base-protecting groups was initially carried out using conc. aq. ammonia at 60° C. for 8 h and a 1:1 mixture of 40% aq. methylamine and conc·aq. ammonia at 60° C. for 2 h.

### Oligonucleotide 40 Synthesis and Kinetics of 3′-Dephosphorylation

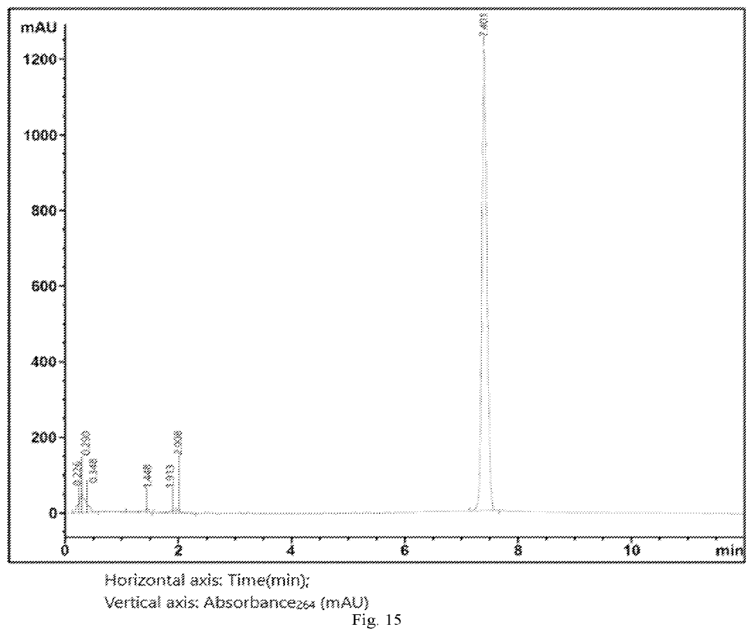

The crude oligonucleotides were analyzed by RP and IE HPLC to reveal the yields and the purity of oligonucleotides synthesized on universal solid support 941c equal or better than that of oligonucleotides synthesized on universal solid support L. A RP HPLC profile of crude reaction mixture of oligonucleotide 40, reproduced below, assembled on universal solid support 941c shown in FIG. 15.

40:
5′-DMT-d(TGT GAG TAC CAC TGA TTA)

Next, kinetics of the 3′-dephosphorylation of a model oligonucleotide 5′-T₁₀-3′ (41) assembled on universal solid support 941 was studied in aqueous methylamine of various concentrations at 25.0, 35.0, and 45.0° C. under the pseudo-first order conditions. The disappearance of the starting material, the oligonucleotide released from the solid phase with the linker still attached at the 3′-terminus was followed by RP HPLC for a period of at least 6 half-lives. The product distribution study showed that the 3′-dephosphorylation was the only reaction detected under all tested conditions. Accordingly, the pseudo first-order rate constants for the disappearance of the starting material were obtained by applying the integrated first-order rate equation to the time-dependent diminution of the concentration of the starting material. Similarly, the rate constants for the accumulation of the product, 5′-T₁₀-3′, were obtained. As the data was collected at three different temperatures, the thermodynamic constants of the process were readily extracted.

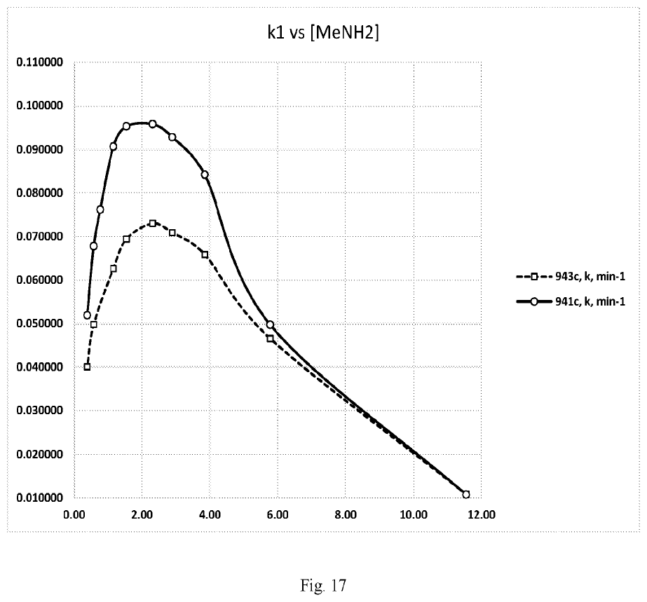

The 3′-dephosphorylation of 41 was strictly first order in the starting material and in the accumulation of the product. Not intending to be bound by theory, conducting the process at ten different concentrations of aqueous methylamine ranging from 11.55 M to 0.39 M (40% and 30-fold diluted methylamine, respectively) revealed a complex relationship between the base concentration and the rate constants (FIG. 17). The reaction rates reached their maximum in the range of methylamine concentrations of 1.03 to 3.42 M (10- and 3-times dilution, respectively). In the optimal range, half-lives of disappearance of the starting material at 45° C. were in the range of 7 to 8 min, while in 40% aq. methylamine the pseudo-first order rate constant decreased by a factor of 8.7, with the half-life of 64.5 min.

3′-Dephosphorylation of oligonucleotides containing 2′-OMe-modified nucleotide residue at the 3′-terminus by universal solid support 941c was tested using 5′-T₉ (2′-OMeU)-3′ as a model compound. As compared with 41, the rate of 3′-dephosphorylation was accelerated by a factor of 2.0 to 2.2 depending on the dilution, with the optimal concentration of methylamine and the optimal concentration window remaining in the same range.

When oligonucleotide 41 was connected to universal solid support 941c via a phosphorothioate linkage as opposed to the phosphate linkage used in the experiments disclosed above, the rate of 3′-dephosphorylation was retarded by 30 to 40% depending on the dilution, with the optimal concentration of methylamine and the optimal concentration window remaining in the same range.

In yet another experiment, universal phosphoramidite 878 was coupled to hydroxypropyl CPG1000 on 1 μmol scale using the conventional coupling cycle of the instrument. The solid support obtained was used for the assembly of oligonucleotide 40. The following deprotection yielded the crude oligonucleotide whose HPLC profiles were indistinguishable from those obtained by conducting the synthesis on universal solid support 941c.

### Influence of Substituents at the N-8 of the Tropane Ring

To assess the influence of the substituent at the N-8 of the tropane ring, oligonucleotide 41 was assembled on universal solid support 943c. The concentration-dependent pseudo-first order rate constants for 3′-dephosphorylation of 41 were obtained as disclosed for universal solid support 941c above. It was found that the 3′-dephosphorylation of 41 was retarded by ca. 30%, with the optimal concentration of methylamine and the optimal concentration window remaining in the same range (FIG. 17). Not intending to be bound by theory, it is conceivable that the retardation results from the steric bulk of N-isopropyl group. However, universal solid support 943c remains quite efficient with the half-life of 9.5 min at 45° C.

Not intending to be bound by theory, the performance of universal solid support 941 was very close to that of universal solid support L in 40% methylamine, while in 3- to 10-times diluted methylamine the 3′-dephosphorylation on universal solid support 941 was about 9 times as rapid as that on universal solid support L.

### Example 4: Developing the Deprotection Conditions Optimal for Both Nucleic Bases and Universal Solid Support 941

To develop the deprotection conditions optimal for both nucleic bases and universal solid support 941, the kinetics of deprotection of model oligonucleotides containing 1, 3, and 6 residues of dG^{ib} were studied. To this end the following four model oligonucleotides 42-45 were designed with the following considerations:
a. dG^{ib} nucleotide residue was selected because it displays the slowest deprotection kinetics among all other protected nucleotide residues;
b. dG-rich oligonucleotides form complex inter- and intramolecular structures, for instance dG-quadruplet. There is no information regarding the properties of partially protected oligonucleotides in solution. Not intending to be bound by theory, if said structures were formed by partially protected oligonucleotides, their presence could influence the rates of deprotection of said oligonucleotides. To see whether such influence exists, oligonucleotides 42 and 43 featured dG^{ib} nucleotide residues separated by those of thymidine as in 43 and the residues clustered together as in 44 and 45.
c. Long dG tracts may display unexpected physico-chemical properties. To see whether their presence may retard the progress of deprotection, the compound 45 featured six dG^{ib} nucleotide residues in a row.
d. The presence of negatively-charged phosphate backbone in oligonucleotides often retards reactions of said oligonucleotides with nucleophilic agents. To exclude unnecessary variability of rate constants due to potential more rapid deprotection of terminal dG^{ib} nucleotide residues, all dG^{ib} residues in 42-45 were placed internally.
   Solid-Support Synthesis of Oligonucleotides 42-45 and their Deprotection Kinetics

Compounds 42-45 were assembled on the conventional nucleosidic solid support DMT-T CPG1000. The 5′-DMT group was removed at the end of the synthesis, and all 2-cyanoethyl protecting groups were removed post-synthetically by treatment with 0.5 M piperidine in acetonitrile for 15 min. Vacuum-dried, solid support-bound oligonucleotides 42-45 were stored at −80° C. as needed:

42:
d(TTT TG^{ib}T TTT T);
43:
d(TG^{ib}T G^{ib}TG^{ib} TTT T);
44:
d(TTT G^{ib}G^{ib}G^{ib} TTT T);
45:
d(TTG^{ib} G^{ib}G^{ib}G^{ib} G^{ib}G^{ib}T T).

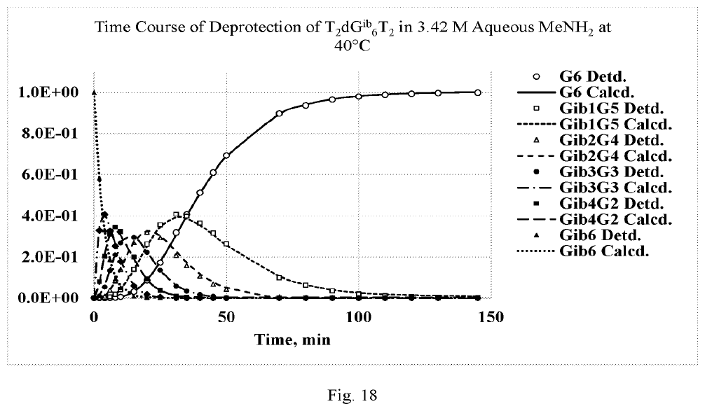

Oligonucleotides 42-45 were subjected to deprotection with 1.37, 2.56, and 3.42 M aqueous methylamine (dilutions of 7.5, 4.0, and 3.0 times, respectively) at 30.0, 40.0, and 50.0° C. The fate of the completely base-protected starting material released to the solution from the solid phase, all incompletely deprotected intermediates, and the final products, completely base-deprotected oligonucleotides, was followed by RP HPLC. The time-dependent product distribution in the reaction mixtures of compound 45 in 3.42 M methylamine at 40° C. is shown in FIG. 18. The pseudo first-order rate constants for the disappearance of the starting material were obtained by applying the integrated first-order rate equation to the time-dependent diminution of the concentration of the starting material. For the extraction of rate constants for the following steps in the reaction sequence except for the last step, differential rate equation (1) was applied and fitted to experimental data by numerical integration. For accumulation of completely deprotected 45, differential rate equation (2) was used and fitted to experimental data by numerical integration.

$$[ {( {G}^{i b} )}_{m} {G}_{n} ] = {\int }_{0}^{t} ( {k}_{( m + 1 )} [ {( {G}^{i b} )}_{( m + 1 )} {G}_{n - 1} ] - {k}_{m} [ {( {G}^{i b} )}_{m} {G}_{n} ] dt , \qquad ( 1 )$$

$$where m and n are integers 2 \leq m \leq 5 , m + n = 6 ;$$

$$[ {G}_{6} ] = {\int }_{0}^{t} {k}_{1} [ {G}^{i b} {G}_{5} ] d t + {[ {G}_{6} ]}_{0} \qquad ( 2 )$$

Analysis of the kinetic data revealed that each step of deprotection for all oligonucleotides tested obeyed the first order law with respect to the starting material, partially deprotected intermediates, and the deprotection agent, methylamine. The rate constants for each step of deprotection were normalized on the number of the residues available for deprotected in that step by equation (3).

$$Normalized {k}_{n} = \frac{Apparent kn}{number of Gib} \qquad ( 3 )$$

It was also found that the normalized rate constants for all deprotection steps were essentially independent of the number of dG^{ib} residues or their placement in the oligonucleotide. This feature permitted the extraction of energy parameters for the removal of isobutyryl residue.

TABLE 2
Activation energy for deprotection of dG^{ib} of the oligonucleotides 42-
45 and that of dephosphorylation of oligonucleotide 41 by USS 941.

| | T₄dG^{ib}T₅, | T₃dG^{ib}₃T4, | (TdG^{ib})₃T4, | T₂dG^{ib}₆T₂, | Dephosphorylation |
|---|---|---|---|---|---|
| | 42 | 43 | 44 | 45 | of T₁₀ on USS 941 |
| Ea, | 61.2 | 62.2 | 61.8 | 57.1 | 39.9 |
| kJ/mol | | | | | |

As seen in Table 2, the activation energies determined for deprotection of a single dG^{ib} residue in oligonucleotides 42-45 is 1.4 to 1.5 times higher than that for the 3′-dephosphorylation by USS 941 when said universal solid support 941 is used under the optimal conditions.

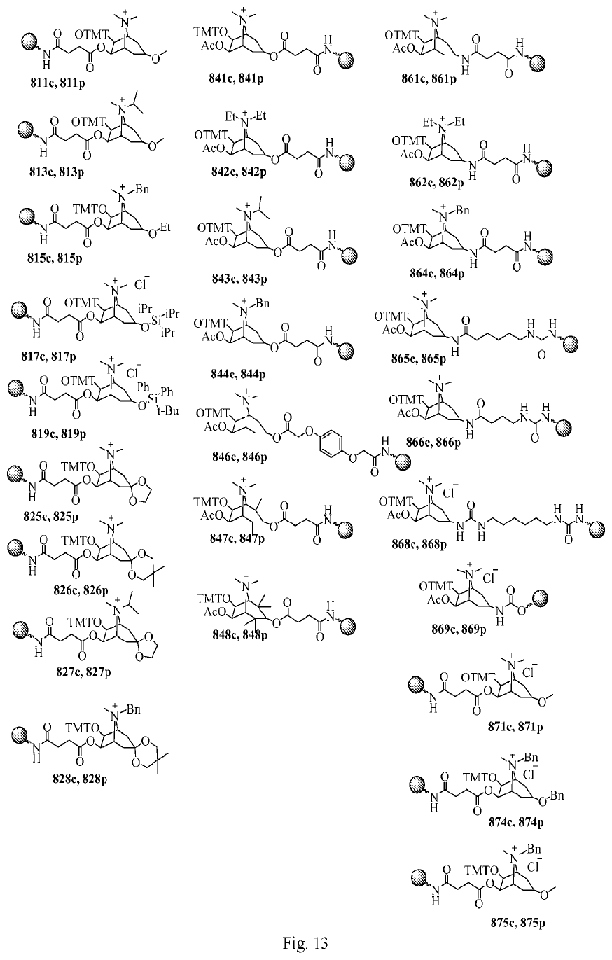

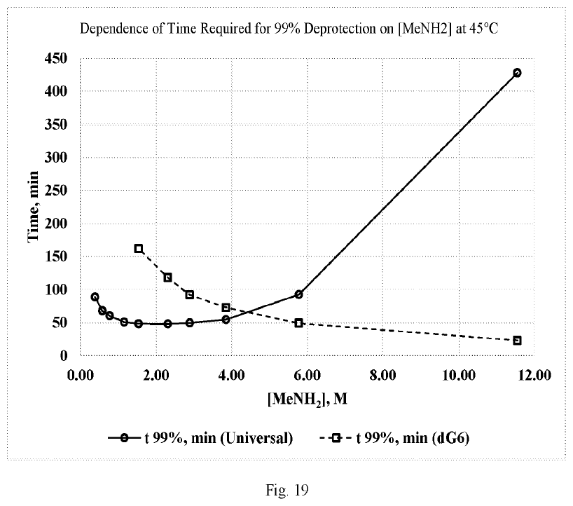

Based on the obtained kinetic and thermodynamic data, optimization of the deprotection conditions was carried out with the objective to determine the concentration of methylamine at which oligonucleotide 45 would be deprotected to the extent wherein only 1% of one out six dG residues would remain protected (0.167% per dG residue) while the oligonucleotide would be 3′-dephosphorylated to the same 99% extent. The plot shown in FIG. 13 shows that, with the increasing concentration of methylamine, the time required for 99% 3′-dephosphorylation by universal solid support 941 increases, while that for oligonucleotide 45 decreases. The intersection of the two curves represents the optimal concentration of methylamine of about 4.5 M and deprotection time of 70-75 min at 45° C. (FIG. 19).

As the optimal deprotection time and concentration of methylamine is generated for oligonucleotides comprising six dG residues and, not intending to be bound by theory, assuming that in average said residues are present in 25% ratio, it is believed that such conditions satisfy the majority of synthetic oligonucleotides of 20-25 nt residues in length most commonly used in the art.

Accordingly, the preparation of novel universal solid supports synthesis and universal phosphoramidite building blocks for oligonucleotide synthesis and their use in assembling of oligonucleotides and their deprotection under the optimized conditions disclosed herein is demonstrated. Beneficially for the overall deprotection time, the 3′-dephosphorylation of oligonucleotides under said conditions proceeds more rapidly than the deprotection of N-isobutyryl group in the residues of guanidine.

### Example 5

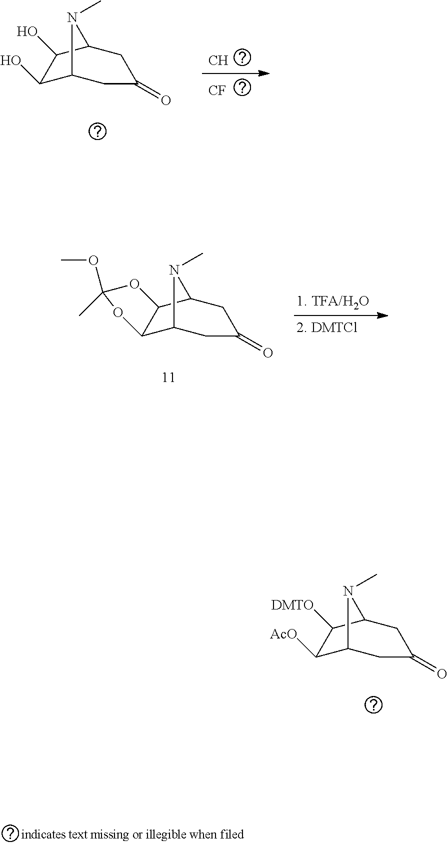

### rel-(1R,5S,6S,7R)-7-[(Bis(4-methoxyphenyl)phenyl)methoxy]-8-methyl-3-oxo-8-azabicyclo[3.2.1]octan-6-yl acetate, 131

Compound 1 (4.9 g, 28.62 mmol) prepared as disclosed in J. Heterocyclic Chem, 29, 1541, 1992), (J. Am. Chem. Soc., 74, 3825-3828, 1952 was suspended in acetonitrile (40 mL) and trimethyl orthoacetate (5.2 g, 43 mmol) and was placed in an oil bath at 45° C. Trifluoracetic acid (0.6 mL, 15.2 mmol) was added, and the reaction mixture was stirred for 30 min. TLC analysis confirmed the complete conversion of the starting material to the corresponding orthoester derivative. The reaction mixture was removed from the heating bath and was cooled to room temperature. Water (0.772 g, 43 mmol) was added and the reaction was stirred for 1 h, after which time the hydrolysis of the orthoester 11 to the respective monoacetate was complete by TLC analysis. The reaction was quenched with 10% aq NaHCO₃ (10 mL) and volatiles were evaporated under vacuum. The residue obtained was dissolved in CH₂Cl₂ (50 mL) and was washed with a 1:1 mixture of 10% aq NaHCO₃ and brine (50 mL). The organic extract was dried (sodium sulfate) and evaporated to dryness. The oily solid was triturated with hexane and dried under high vacuum to yield compound 131 as a solid foam (5.2 g, 85%).

¹H-NMR (500 MHz, CDCl₃) δ 4.78 (1H, d); 4.1 (1H, d); 3.52 (1H, s); 3.51 (1H, s); 3.04 (1H, m) 2.53 (2H, m); 2.52 (3H, s); 2.3 (2H, m); 1.07 (6H, d). ¹³C-NMR (100 MHz, CDCl₃) δ 20.9, 34.38; 40.49; 40.83; 64.72; 67.89; 75.21; 170.65; 206.19. ES MS: [M+H]⁺ 214.2 (observed), 213.1 (calcd.).

The product of the previous step (2.2 g, 10.3 mmol) in CH₂Cl₂ (40 mL) and iPr₂NEt (4 g, 31 mmol) was treated with dimethoxytrityl chloride (3.85 g, 11.35 mmol). The reaction mixture was stirred for 2 h, diluted with CH₂Cl₂ (40 mL), and washed with 10% aq NaHCO₃. The organic extract was dried (sodium sulfate) and was evaporated under vacuum. The residue was dissolved in methanol (100 mL) and cooled in an ice-water bath. Upon magnetic stirring for 30 min, the desired product precipitated from the solution. The product was filtered off and was redissolved in CH₂Cl₂ (20 mL). The solution was evaporated, and the oily residue was dried in vacuo to give the product as a solid foam (4.4 g, 82%).

¹H-NMR (500 MHz, DMSO-d₆) δ 7.3 (m, 2H); 7.25 (m, 2H); 7.23 (m, 5H); 6.9 (m, 4H); 4.64 (d, 1H); 3.79 (d, 1H); 3.78 (s, 6H); 3.32 (s, 2H); 2.62 (m, 2H); 2.5 (m, 2H); 2.44 (m, 1H); 2.09 (s, 3H); 1.09 (d, 1H); 1.50 (d, 1H). ¹³C-NMR (100 MHz, DMSO-d₆) δ 20.67, 21.01, 35.7, 41.47, 42.18, 55.05, 65.31, 65.61, 77.03, 77.66, 87.08, 113.25, 126.84, 127.79, 127.83, 129.71, 129.78, 135.91, 136.12, 145.27, 158.22, 158.25, 169.86, 206.73. ES MS: [M+H]⁺ 516.1 (observed), 515.2 (calcd.).

### Example 6

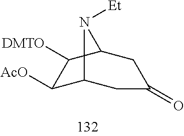

### rel-(1R,5S,6S,7R)-7-(Bis(4-methoxyphenyl)(phenyl)methoxy)-8-ethyl-3-oxo-8-azabicyclo[3.2.1]octan-6-yl acetate, 132

Compound 132 was synthesized by following the procedure described in Example 1 above.

ES MS: [M+H]⁺ 530.2 (observed), 530.6 (calcd.).

### Example 7

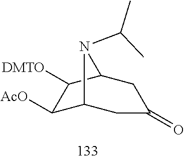

### rel-(1R,5S,6S,7R)-7-(Bis(4-methoxyphenyl)(phenyl)methoxy)-8-isopropyl-3-oxo-8-azabicyclo[3.2.1]octan-6-yl acetate, 133

Compound 133 was synthesized by following the procedure described in Example 5 above.

¹H-NMR (500 MHz, DMSO-d₆) δ 7.4 (m, 2H); 7.26 (m, 2H); 7.23 (m, 5H); 6.9 (m, 4H); 4.72 (m, 1H); 3.74 (s, 1H); 3.72 (s, 6H); 3.57 (s, 1H); 3.32 (s, 1H); 2.96 (m, 1H); 2.74 (s, 1H); 2.24 (m, 1H); 2.13 (s, 3H); 1.99 (m, 1H); 1.47 (d, 1H); 1.02 (s, 3H); 0.9 (s, 3H). ¹³C-NMR (100 MHz, DMSO-d₆) δ 21.39, 21.56, 41.25, 41.94, 44.47, 55.06, 60.91, 61.33, 76.59, 77.05, 87.0, 113.26, 126.80, 127.78, 127.83, 129.66, 129.77, 136.01, 136.17, 145.31, 158.20, 158.25, 170.01, 207.16. ES MS: [M+H]⁺ 544.2 (observed), 544.7 (calcd.).

### Example 8

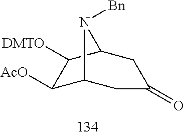

### rel-(1R,5S,6S,7R)-7-(Bis(4-methoxyphenyl)(phenyl)methoxy)-8-benzyl-3-oxo-8-azabicyclo[3.2.1]octan-6-yl acetate, 134

Compound 134 was synthesized by following the procedure described in Example 1 above.

ES MS: [M+H]⁺ 592.3 (observed), 592.7 (calcd.).

### Example 9

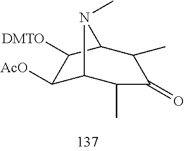

### rel-(1R,5S,6S,7R)-7-(Bis(4-methoxyphenyl)(phenyl)methoxy)-2,4,8-trimethyl-3-oxo-8-azabicyclo[3.2.1]octan-6-yl acetate, 137

Compound 137 was synthesized by following the procedure described in Example 1 above.

ES MS: [M+H]⁺ 544.1 (observed), 544.7 (calcd.).

### Example 10

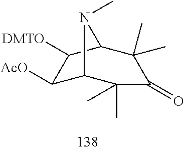

### rel-(1R,5S,6S,7R)-7-(Bis(4-methoxyphenyl)(phenyl)methoxy)-2,2,4,4,8-pentamethyl-3-oxo-8-azabicyclo[3.2.1]octan-6-yl acetate, 138

Compound 138 was synthesized by following the procedure described in Example 1 above.

ES MS: [M+H]⁺ 572.9 (observed), 572.7 (calcd.).

### Example 11

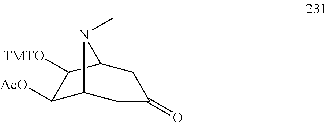

### rel-(1R,5S,6S,7R)-7-(Tris(4-methoxyphenyl)methoxy)-8-ethyl-3-oxo-8-azabicyclo[3.2.1]octan-6-yl acetate, 231

Compound 231 was synthesized by following the procedure described in Example 1 above.

ES MS: [M+H]⁺ 548.0 (observed), 547.7 (calcd.).

### Example 12

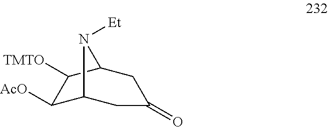

### rel-(1R,5S,6S,7R)-7-(Tris(4-methoxyphenyl)methoxy)-8-ethyl-3-oxo-8-azabicyclo[3.2.1]octan-6-yl acetate, 232

Compound 232 was synthesized by following the procedure described in Example 1 above.

ES MS: [M+H]⁺ 561.2 (observed), 561.7 (calcd.).

### Example 13

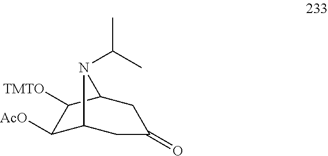

### rel-(1R,5S,6S,7R)-7-(Tris(4-methoxyphenyl)methoxy)-8-isopropyl-3-oxo-8-azabicyclo[3.2.1]octan-6-yl acetate, 233

ES MS: [M+H]⁺ 575.4 (observed), 575.7 (calcd.).

### Example 14

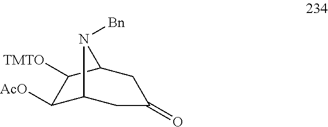

### rel-(1R,5S,6S,7R)-7-(Tris(4-methoxyphenyl)methoxy)-8-benzyl-3-oxo-8-azabicyclo[3.2.1]octan-6-yl acetate, 234

Compound 334 was synthesized by following the procedure described in Example 1 above.

ES MS: [M+H]⁺ 624.1 (observed), 623.7 (calcd.).

### Example 15

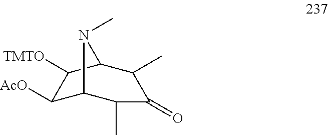

### rel-(1R,5S,6S,7R)-7-(Tris(4-methoxyphenyl)methoxy)-2,4,8-trimethyl-3-oxo-8-azabicyclo[3.2.1]octan-6-yl acetate, 237

Compound 237 was synthesized by following the procedure described in Example 1 above.

ES MS: [M+H]⁺ 575.1 (observed), 575.7 (calcd.).

### Example 16

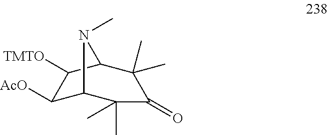

### rel-(1R,5S,6S,7R)-7-(Tris(4-methoxyphenyl)methoxy)-2,4,8-trimethyl-3-oxo-8-azabicyclo[3.2.1]octan-6-yl acetate, 238

Compound 238 was synthesized by following the procedure described in Example 1 above.

ES MS: [M+H]⁺ 602.9 (observed), 603.8 (calcd.).

### Example 17

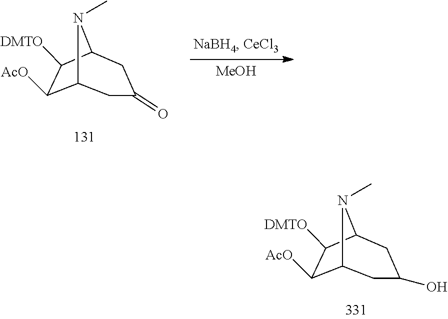

### rel-(1R,5S,6S,7R)-7-(Bis(4-methoxyphenyl)(phenyl)methoxy)-3-hydroxy-8-methyl-8-azabicyclo[3.2.1]octan-6-yl acetate, 331

Compound 131 (4.2 g, 8.15 mmol) in methanol (50 mL) was cooled in an ice bath. To this, NaBH₄ (12.22 mmol, 0.46 g) and CeCl₃ (50 mg, 0.2 mmol) were added gradually over 15 min. The reaction mixture was stirred for 30 min and was quenched with saturated NH₄Cl solution (10 mL). The volatiles were evaporated under vacuum, the residue was redissolved in CH₂Cl₂ (50 ml) and washed with water (50 mL) and brine (50 mL). The organic extract was dried over sodium sulfate and evaporated. The crude mixture was purified by column chromatography (CH₂Cl₂-MeOH), which afforded the desired compound 331 as a white foam (3.5 g, 83%).

¹H-NMR (500 MHz, DMSO-d₆) δ 7.4 (m, 2H); 7.27 (m, 7H); 6.87 (m, 4H); 4.6 (m, 1H); 4.49 (d, 1H); 3.97 (d, 1H); 3.74 (s, 6H); 3.13 (m, 1H); 2.94 (s, 1H); 2.53 (s, 1H); 2.35 (s, 3H); 1.99 (s, 3H); 1.5 (m, 1H); 1.33 (m, 1H); 1.16 (m, 2H). ¹³C-NMR (100 MHz, DMSO-d₆) δ 21.09, 33.5, 33.8, 37.02, 54.87, 55.04, 62.01, 65.55, 65.80, 76.92, 77.25, 87.04, 113.13, 126.75, 127.74, 127.98, 129.84, 129.91, 136.23, 136.38, 145.47, 158.14, 169.76. ES MS: [M+Na]⁺ 517.25 (observed), 518.4 (calcd.) [M+Na]⁺ 540.2, [2M+Na]⁺ 1057.2 (observed).

### Example 18

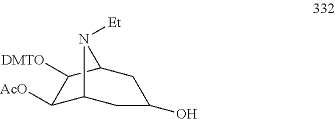

### rel-(1R,5S,6S,7R)-7-(Bis(4-methoxyphenyl)(phenyl)methoxy)-3-hydroxy-8-ethyl-8-azabicyclo[3.2.1]octan-6-yl acetate, 332

The compound 332 was synthesized by following the procedure described in Example 17 above.

ES MS: [M+H]⁺ 532.6 (observed), 532.6 (calcd.) [M+Na]⁺ 554.3 (observed).

### Example 19

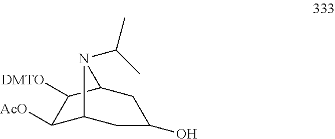

### rel-(1R,5S,6S,7R)-7-(Bis(4-methoxyphenyl)(phenyl)methoxy)-3-hydroxy-8-isopropyl-8-azabicyclo[3.2.1]octan-6-yl acetate, 333

The compound 333 was synthesized by following the procedure described in Example 17 above.

¹H-NMR (500 MHz, DMSO-d₆) δ 7.4 (m, 2H); 7.26 (m, 7H); 6.9 (m, 4H); 4.69 (d, 1H); 4.45 (d, 1H); 3.88 (m, 1H); 3.73 (s, 6H); 3.23 (s, 1H); 3.14 (s, 1H); 2.98 (m, 1H); 2.66 (s, 1H); 2.04 (s, 3H); 1.49 (s, 1H); 1.26 (m, 1H); 1.19 (m, 2H), 0.93 (s, 3H); 0.84 (s, 3H). 13C-NMR (100 MHz, DMSO-d₆) δ 21.13, 2.42, 21.59, 32.89, 33.11, 44.34, 55.04, 60.59, 60.90, 62.11, 76.58, 76.63, 86.88, 113.14, 126.7, 127.74, 127.94, 129.76, 129.86, 136.37, 136.45, 145.56, 158.11, 158.14, 169.96. ES MS: [M+H]⁺ 546.9 (observed), 546.7 (calcd.) [M+Na]⁺ 568.4 (observed).

### Example 20

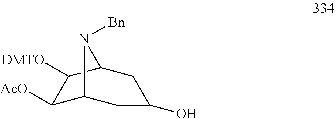

### rel-(1R,5S,6S,7R)-7-(Bis(4-methoxyphenyl)(phenyl)methoxy)-3-hydroxy-8-benzyl-8-azabicyclo[3.2.1]octan-6-yl acetate, 334

The compound 334 was synthesized by following the procedure described in Example 17 above.

ES MS: [M+H]⁺ 594.3 (observed), 594.7 (calcd.) [M+Na]⁺ 616.2 (observed).

### Example 21

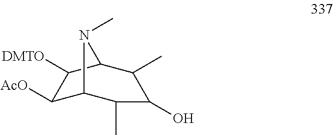

### rel-(1R,5S,6S,7R)-7-(Bis(4-methoxyphenyl)(phenyl)methoxy)-2,4,8-trimethyl-3-hydroxy-8-azabicyclo[3.2.1]octan-6-yl acetate, 337

Compound 337 was synthesized by following the procedure described in Example 17 above.

ES MS: [M+H]⁺ 546.9 (observed), 546.7 (calcd.).

### Example 22

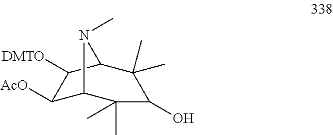

### rel-(1R,5S,6S,7R)-7-(Bis(4-methoxyphenyl)(phenyl)methoxy)-2,2,4,4,8-pentamethyl-3-hydroxy-8-azabicyclo[3.2.1]octan-6-yl acetate, 338

Compound 338 was synthesized by following the procedure described in Example 17 above.

ES MS: [M+H]⁺ 574.0 (observed), 574.7 (calcd.).

### Example 23

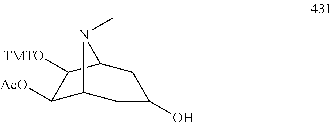

### rel-(1R,5S,6S,7R)-7-(Tris(4-methoxyphenyl)methoxy)-3-hydroxy-8-methyl-8-azabicyclo[3.2.1]octan-6-yl acetate, 431

The compound 431 was synthesized by following the procedure described in Example 17 above.

ES MS: [M+H]⁺ 550.2 (observed), 549.7 (calcd.) [M+Na]⁺ 582.1 (observed).

### Example 24

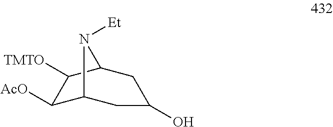

### rel-(1R,5S,6S,7R)-7-(Tris(4-methoxyphenyl)methoxy)-3-hydroxy-8-ethyl-8-azabicyclo[3.2.1]octan-6-yl acetate, 432

The compound 432 was synthesized by following the procedure described in Example 17 above.

ES MS: [M+H]⁺ 564.1 (observed), 563.7 (calcd.) [M+Na]⁺ 586.8 (observed).

### Example 25

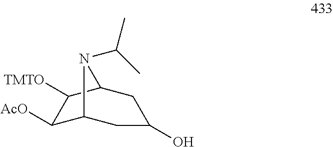

### rel-(1R,5S,6S,7R)-7-(Tris(4-methoxyphenyl)methoxy)-3-hydroxy-8-isopropyl-8-azabicyclo[3.2.1]octan-6-yl acetate, 433

The compound 433 was synthesized by following the procedure described in Example 17 above.

ES MS: [M+H]⁺ 577.6 (observed), 577.7 (calcd.) [M+Na]⁺ 599.9 (observed).

### Example 26

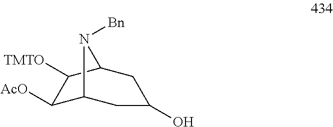

### rel-(1R,5S,6S,7R)-7-(Tris(4-methoxyphenyl)methoxy)-3-hydroxy-8-benzyl-8-azabicyclo[3.2.1]octan-6-yl acetate, 434

The compound 434 was synthesized by following the procedure described in Example 17 above.

ES MS: [M+H]⁺ 626.6 (observed), 625.7 (calcd.) [M+Na]⁺ 647.1 (observed).

### Example 27

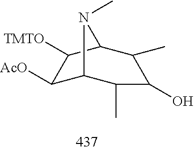

### rel-(1R,5S,6S,7R)-7-(Tris(4-methoxyphenyl)methoxy)-2,4,8-trimethyl-3-hydroxy-8-azabicyclo[3.2.1]octan-6-yl acetate, 437

Compound 437 was synthesized by following the procedure described in Example 17 above.

ES MS: [M+H]⁺ 578.4 (observed), 775.7 (calcd.).

### Example 28

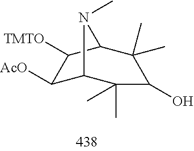

### rel-(1R,5S,6S,7R)-7-(Tris(4-methoxyphenyl)methoxy)-2,2,4,4,8-pentamethyl-3-hydroxy-8-azabicyclo[3.2.1]octan-6-yl acetate, 438

Compound 438 was synthesized by following the procedure described in Example 17 above.

ES MS: [M+H]⁺ 606.5 (observed), 605.8 (calcd.).

### Example 29

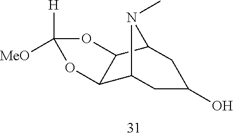

### 9-Methyl-2-methoxy-hexahydro-6H-cyclohepta-1,3-dioxol-4,8-imin-6-ol, 31

Compound 31 was synthesized from compound 26 by following the procedure described in Example 17 above.

ES MS: [M+H]⁺ 215.7 (observed), 216.2 (calcd.).

### Example 30

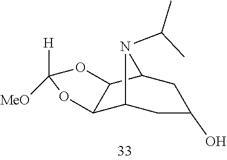 [SMILES: COC1OC2C(O1)C1CC(O)CC2N1C(C)C]

### 9-Isopropyl-2-methoxy-hexahydro-6H-cyclohepta-1,3-dioxol-4,8-imin-6-ol, 33

Compound 33 was synthesized from compound 27 by following the procedure described in Example 17 above.

ES MS: [M+H]⁺ 244.9 (observed), 244.3 (calcd.).

### Example 31

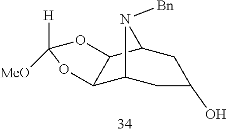 [SMILES: COC1OC2C(O1)C1CC(O)CC2N1Cc1ccccc1]

### 9-Benzyl-2-methoxy-hexahydro-6H-cyclohepta-1,3-dioxol-4,8-imin-6-ol, 34

Compound 34 was synthesized from compound 28 by following the procedure described in Example 17 above.

ES MS: [M+H]⁺ 291.5 (observed), 292.3 (calcd.).

### Example 32

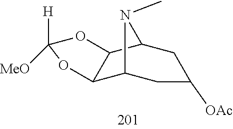 [SMILES: COC1OC2C(O1)C1CC(OC(C)=O)CC2N1C]

### (9-Methyl-2-methoxy-hexahydro-6H-cyclohepta-1,3-dioxol-4,8-imin-6-yl) acetate, 201

Acetic anhydride (g, 30 mmol) was added to magnetically stirred solution of compound 31 (5.14 g, 20 mmol) in pyridine (30 mL) under cooling in an ice bath. The reaction mixture was stirred at room temperature overnight to show the complete disappearance of the starting material by TLC. The excess acylating agent was quenched by adding water (1.8 mL, 100 mmol) followed by TEA (4.04 g, 40 mmol). The mixture was stirred at room temperature for 2 h and was evaporated to an oil. The residue was dissolved in ethyl acetate (100 mL), washed with 5% aq. NaHCO₃ (2×20 mL) and with brine (20 mL). The organic phase was dried over Na₂SO₄, evaporated to an oil, and coevaporated with acetonitrile (2×25 mL). The residue was dissolved in ethyl acetate (10 mL) and added, in a slow rate to ice-cold hexanes (100 mL), which resulted in precipitation of the desired product as a colorless thick oil. The solution was decanted, and the residue was dried on an oil pump to yield the desired product as a ca. 10:1 mixture of diastereomers, a colorless thick oil (6.71 g, 87%).

¹H-NMR (500 MHz, DMSO-d₆) δ 5.97+5.70 (1H, s, 1:10), 5.28 (1H, m), 4.26+4.46 (2H, s, 1:10), 3.29+3.43 (3H, s, 10:1); 2.43 (s, 3H), 2.23 (2H, s), 2.12 (2H, m), 1.77 (3H, s), 1.47 (2H, m). ES MS: [M+H]⁺ 257.4 (observed), 258.3 (calcd.).

### Example 33

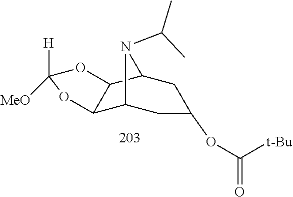 [SMILES: COC1OC2C(O1)C1CC(OC(=O)C(C)(C)C)CC2N1C(C)C]

### (9-Isopropyl-2-methoxy-hexahydro-6H-cyclohepta-1,3-dioxol-4,8-imin-6-yl) pivalate, 203

Compound 203 was synthesized from compound 33 by following the procedure described in Example 32 above.

ES MS: [M+H]⁺ 239.1 (observed), 238.4 (calcd.).

### Example 34

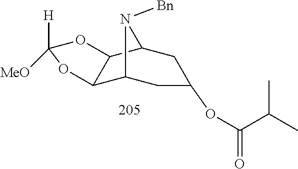 [SMILES: COC1OC2C(O1)C1CC(OC(=O)C(C)C)CC2N1Cc1ccccc1]

### (9-Benzyl-2-methoxy-hexahydro-6H-cyclohepta-1,3-dioxol-4,8-imin-6-yl) isobutyrate, 205

Compound 205 was synthesized from compound 34 by following the procedure described in Example 32 above.

ES MS: [M+H]⁺ 361.8 (observed), 362.4 (calcd.).

### Example 35

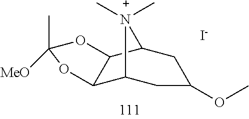 [SMILES: COC1CC2C3OC(C)(OC)OC3C(C1)[N+]2(C)C ; [I-]]

### 9,9-Dimethyl-2,6-dimethoxy-hexahydro-6H-cyclohepta-1,3-dioxol-4,8-iminium iodide, 111

Compound 21 (5.73 g, 25 mmol) was dissolved in anhydrous DMF (20 mL) containing sodium hydride (60% suspension in oil, 1.05 g, 26.25 mmol). The mixture was magnetically stirred in a pressure flask for 30 min at 60° C. The flask was brought to room temperature, and methyl iodide (14.2 g, 100 mmol) was added. The mixture was stirred at 45° C. overnight, after which time the reaction was found complete by TLC. The mixture was evaporated in vacuo and co-evaporated with toluene (5×50 mL). The residue was suspended in anhydrous acetone (50 mL), heated with stirring at 45° C. for 20 min, and was cooled to 0° C. The liquid phase was removed, and the treatment was repeated. The solid was filtered off, washed with cold acetone, and dried in vacuo to give the desired product as an off-white solid (g, 73.2%).

¹H-NMR (500 MHz, DMSO-d₆) δ 6.12+5.93 (total 1H, s, 1:10), 4.34+4.58 (total 2H, s), 3.60+3.58 (3H, s, 10:1); 3.41 (3H, s), 3.30 (3H, s), 3.25 (3H, s), 3.08 (1H, m), 2.31 (2H, m), 2.19 (2H, m). ES MS: [M+H]⁺ 257.4 (observed), 258.3 (calcd.).

### Example 36

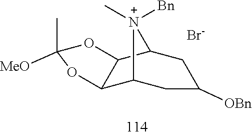 [SMILES: COC1(C)OC2C(O1)C1CC(OCc3ccccc3)CC2[N+]1(C)Cc1ccccc1 ; [Br-]]

### 9-methyl-9-benzyl-2-methoxy6-benzyloxy-hexahydro-6H-cyclohepta-1,3-dioxol-4,8-iminium iodide, 114

Compound 114, an off-white solid (58.5%), was synthesized from compound 21 by following the procedure described in Example 32. ES MS: [M]⁺ 411.1 (observed), 410.5 (calcd.).

### Example 37

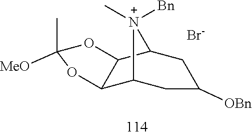 [SMILES: COC1(C)OC2C(O1)C1CC(OCc3ccccc3)CC2[N+]1(C)Cc1ccccc1 ; [Br-]]

### 9-methyl-9-benzyl-2-methoxy-6-benzyloxy-hexahydro-6H-cyclohepta-1,3-dioxol-4,8-iminium bromide, 114

Compound 114, an off-white solid (58.5%), was synthesized from compound 21 by following the procedure described in Example 32. ES MS: [M]⁺ 411.1 (observed), 410.5 (calcd.).

### Example 38

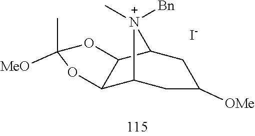 [SMILES: COC1CC2C3OC(C)(OC)OC3C(C1)[N+]2(C)Cc1ccccc1 ; [I-]]

### 9-methyl-9-benzyl-2,6-dimethoxy-hexahydro-6H-cyclohepta-1,3-dioxol-4,8-iminium bromide, 115

Compound 115, an off-white solid (63.6%), was synthesized from compound 24 by following the procedure described in Example 32. ES MS: [M]⁺ 333.6 (observed), 334.4 (calcd.).

### Example 39

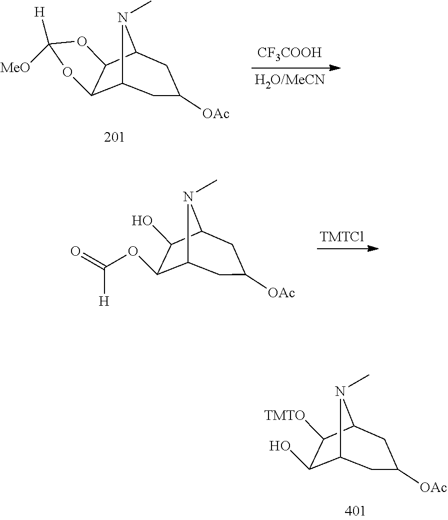

### rel-(1R,5S,6S,7R)-7-[(Tris(4-methoxyphenyl)methoxy]-8-methyl-3-acetoxy-8-azabicyclo[3.2.1]octan-6-ol, 401

Trifluoroacetic acid (0.5 M in anhydrous MeCN, 2.0 mL, 1 mmol) was added to a solution of compound 201 (5.15 g, 20 mmol) in MeCN (50 mL) containing water (0.72 g, 40 mmol). The mixture was kept at room temperature for 6 h, after which time the hydrolysis of 201 to the respective formate ester was found complete. A chromatographic column packed with silica gel (30 mL) was washed with methanol (2 vol., 60 mL) and with anhydrous acetonitrile (2 vol., 60 mL). The reaction mixture was passed through the column while collecting the eluates. The column was then washed with another 2 vol. of acetonitrile. The combined eluates were evaporate to dryness and coevaporated with acetonitrile (3×50 mL). The oily residue was dissolved in anhydrous acetonitrile (20 mL) and DIPEA (3.23 g, 25 mmol), after which solid TMTCl (7.56 g, 20.5 mmol) was added. The reaction mixture was stirred for 3 h at room temperature, and water (1.8 g, 100 mmol) and TEA (2.02 g, 20 mmol) were added. After stirring for another 2 h, solvents were evaporated. The oil obtained was dissolved in ethyl acetate (200 mL), was washed with 5% aq. NaHCO₃ (2×50 mL) and with brine (50 mL). Organic phase was dried over Na₂SO₄, evaporated, and coevaporated with toluene (2×50 mL). The product was isolated by column purification on silica gel using a linear gradient from ether to 5% MeOH in DCM. Evaporation of collected fractions and drying in vacuo gave the desired product as a solid foam (8.62 g, 78.7%). ¹H-NMR (500 MHz, DMSO-d₆) δ 6.08 (m, 6H); 6.07 (m, 6H); 4.60 (m, 1H); 4.49 (d, 1H); 3.97 (d, 1H); 2.94 (s, 1H); 2.26 (s, 3H); 1.99 (s, 3H); 1.70-1.5 (m, 4H); 1.32 (m, 2H). ES MS: [M+H]⁺ 549.3 (observed), 548.6 (calcd.).

### Example 40

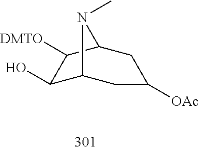

### rel-(1R,5S,6S,7R)-7-[(Bis(4-methoxyphenyl)(phenyl)methoxy]-8-methyl-3-acetoxy-8-azabicyclo[3.2.1]octan-6-ol, 301

Compound 301 isolated as a solid foam (79.5%), was synthesized from compound 201 by following the procedure described in Example 38. ES MS: [M]⁺ 517.8 (observed), 518.6 (calcd.).

### Example 41

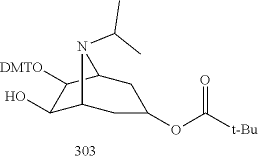

### rel-(1R,5S,6S,7R)-7-[(Bis(4-methoxyphenyl)(phenyl)methoxy]-8-isopropyl-3-pivaloyloxy-8-azabicyclo[3.2.1]octan-6-ol, 303

Compound 301 isolated as a solid foam (79.5%), was synthesized from compound 201 by following the procedure described in Example 38. ES MS: [M]⁺ 588.7 (observed), 588.8 (calcd.).

### Example 42

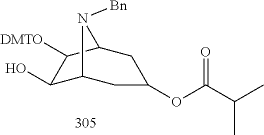

### rel-(1R,5S,6S,7R)-7-[(Bis(4-methoxyphenyl)(phenyl)methoxy]-8-benzyl-3-isobutyryloxy-8-azabicyclo[3.2.1]octan-6-ol, 305

Compound 305 isolated as a solid foam (79.5%), was synthesized from compound 205 by following the procedure described in Example 38. ES MS: [M]⁺ 622.0 (observed), 622.8 (calcd.).

### Example 43

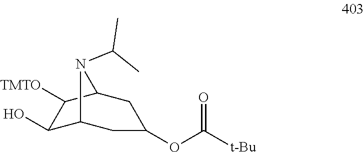

### rel-(1R,5S,6S,7R)-7-[(Tris(4-methoxyphenyl)methoxy]-8-isopropyl-3-pivaloyloxy-8-azabicyclo[3.2.1]octan-6-ol, 403

Compound 403 isolated as a solid foam (81.2%), was synthesized from compound 203 by following the procedure described in Example 38. ES MS: [M]⁺ 620.2 (observed), 619.8 (calcd.).

### Example 44

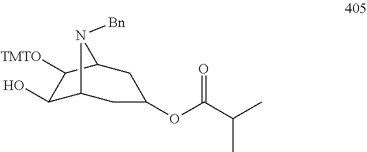

### rel-(1R,5S,6S,7R)-7-[(Tris(4-methoxyphenyl)methoxy]-8-benzyl-3-isobutyryloxy-8-azabicyclo[3.2.1]octan-6-ol, 405

Compound 305 isolated as a solid foam (73.1%), was synthesized from compound 205 by following the procedure described in Example 38. ES MS: [M]⁺ 652.0 (observed), 652.8 (calcd.).

### Example 45

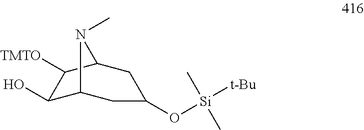

### rel-(1R,5S,6S,7R)-7-[(Tris(4-methoxyphenyl)methoxy]-8-methyl-3-[(t-butyl)dimethylsilyloxy]-8-azabicyclo[3.2.1]octan-6-ol, 416

Compound 416 isolated as a solid foam (72.6%), was synthesized from compound 216 by following the procedure described in Example 38. ES MS: [M]⁺ 620.6, 621.6 (observed), 620.3, 621.3 (calcd.).

### Example 46

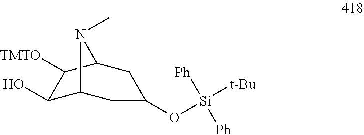

### rel-(1R,5S,6S,7R)-7-[(Tris(4-methoxyphenyl)methoxy]-8-methyl-3-[(t-butyl) diphenylsilyloxy]-8-azabicyclo[3.2.1]octan-6-ol, 418

Compound 418 isolated as a solid foam (69.6%), was synthesized from compound 218 by following the procedure described in Example 38. ES MS: [M]⁺ 744.3, 745.6 (observed), 744.4, 745.4 (calcd.).

### Example 47

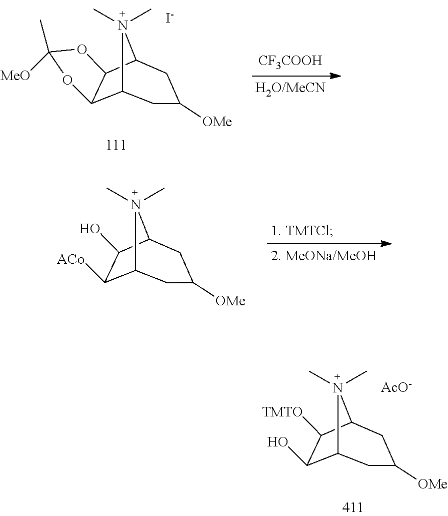

### rel-(1R,5S,6S,7R)-7-[(Tris(4-methoxyphenyl)methoxy]-8,8-dimethyl-3-methoxy-8-azoniabicyclo[3.2.1]octan-6-ol acetate, 411

Trifluoroacetic acid (2.0 g, 1.5 mmol) was added to a solution of compound 211 (5.78 g, 15 mmol) in MeOH (50 mL) containing water (1.08 g, 60 mmol). The mixture was kept at room temperature for 1 h, after which time the hydrolysis of 211 to the respective acetate ester was found complete. The reaction mixture was evaporated to dryness and coevaporated with pyridine (3×50 mL). The oily residue was dissolved in anhydrous pyridine (50 mL) and DIPEA (3.23 g, 25 mmol), after which solid TMTCl (5.90 g, 16 mmol) was added. The reaction mixture was stirred for 3 h at room temperature. Sodium methoxide (30% w/w in MeOH, 8.1 g, 45 mmol) was added. After stirring for another 2 h, excess sodium methoxide was quenched with triethylammonium acetate buffer (1 M, 50 mL), and solvents were evaporated. The oil obtained was dissolved in DCM-MeOH (19:1, 200 mL) and washed with brine (2×50 mL). The organic phase was dried over Na₂SO₄, evaporated, and dissolved in DCM-MeOH (19:1, 60 mL). The product was isolated by column purification on silica gel using a linear gradient from 5% to 40% MeOH in DCM. Collected fractions were evaporated, the residue was dissolved in DCM-MeOH (19:1, 200 mL) and washed with triethylammonium acetate buffer (0.5 M, 2×30 mL). The organic phase was dried over Na₂SO₄, evaporated, and dried in vacuo to yield the desired product as a solid foam (5.40 g, 60.7%). ES MS: [M]⁺ 533.6 (observed), 534.7 (calcd.).

### Example 48

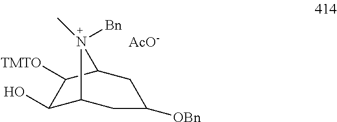

### rel-(1R,5S,6S,7R)-7-[(Tris(4-methoxyphenyl)methoxy]-8-methyl-8-benzyl-3-benzyloxy-8-azoniabicyclo[3.2.1]octan-6-ol acetate, 414

Compound 414 isolated as a solid foam (73.1%), was synthesized from compound 214 by following the procedure described in Example 46. ES MS: [M]⁺ 686.9 (observed), 686.4 (calcd.).

### Example 49

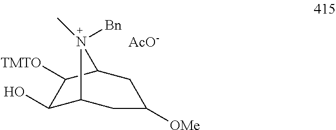

### rel-(1R,5S,6S,7R)-7-[(Tris(4-methoxyphenyl)methoxy]-8-methyl-8-benzyl-3-methoxy-8-azoniabicyclo[3.2.1]octan-6-ol acetate, 415

Compound 414 isolated as a solid foam (65.3%), was synthesized from compound 215 by following the procedure described in Example 46. ES MS: [M]⁺ 611.4 (observed), 610.3 (calcd.).

### Example 50

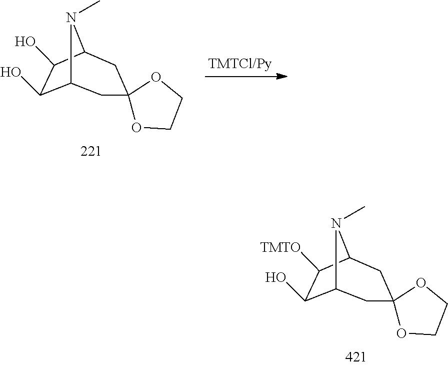

### rel-(1R,5S,6S,7R)-7-[(Tris(4-methoxyphenyl)methoxy]-8-methyl-azaspiro[bicyclo[3.2.1]octane-3,2′-[1,3]dioxolane]-6-ol, 421

Solid TMTCl (5.90 g, 7.95 mmol) was added to compound 221 (1.61 g, 7.5 mmol) in anhydrous pyridine (40 mL) and DIPEA (1.25 g, 9.9 mmol). The reaction mixture was stirred at room temperature overnight and solvents were evaporated. The oil obtained was dissolved in ethyl acetate (200 mL) and washed with 5% aq. NaHCO₃ (2×50 mL) and with brine (50 mL). Organic phase was dried over Na₂SO₄, evaporated, and coevaporated with toluene (2×50 mL). The product was isolated by column purification on silica gel using a linear gradient from ether to 5% MeOH in DCM. Evaporation of collected fractions and drying in vacuo gave the desired product as a solid foam (3.10 g, 75.4%). ES MS: [M+H]⁺ 548.1 (observed), 547.3 (calcd.).

### Example 51

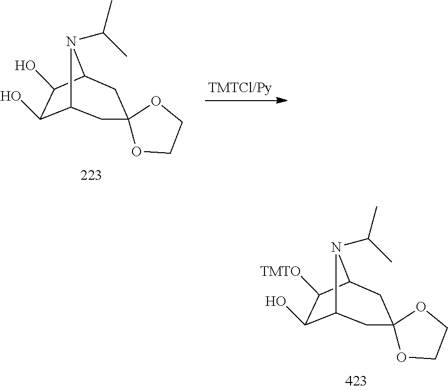

### rel-(1R,5S,6S,7R)-7-[(Tris(4-methoxyphenyl)methoxy]-8-isopropyl-azaspiro[bicyclo[3.2.1]octane-3,2′-[1,3]dioxolane]-6-ol, 423

Compound 423 isolated as a solid foam (62.4%), was synthesized from compound 223 by following the procedure described in Example 50. ES MS: [M+H]⁺ 577.5 (observed), 576.7 (calcd.).

### Example 52

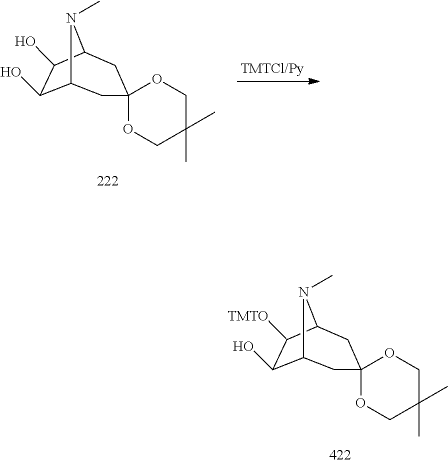

### rel-(1R,5S,6S,7R)-7-[(Tris(4-methoxyphenyl)methoxy]-5′,5′,8-trimethyl-azaspiro[bicyclo[3.2.1]octane-3,2′-[1,3]dioxolane]-6-ol, 422

Solid TMTCl (1.75 g, 4.75 mmol) was added to compound 222 (1.16 g, 4.5 mmol) in anhydrous pyridine (40 mL) and DIPEA (0.69 g, 5.5 mmol). The reaction mixture was stirred at room temperature overnight and solvents were evaporated. The oil obtained was dissolved in ethyl acetate (200 mL) and washed with 5% aq. NaHCO₃ (2×50 mL) and with brine (50 mL). Organic phase was dried over Na₂SO₄, evaporated, and coevaporated with toluene (2×50 mL). The product was isolated by column purification on silica gel using a linear gradient from ether to 5% MeOH in DCM. Evaporation of collected fractions and drying in vacuo gave the desired product as a solid foam (1.93 g, 72.6%). ES MS: [M+H]⁺ 591.5 (observed), 590.7 (calcd.).

### Example 53

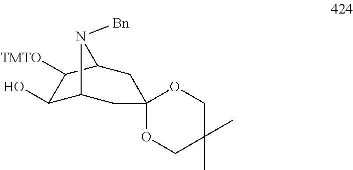

### rel-(1R,5S,6S,7R)-7-[(Tris(4-methoxyphenyl)methoxy]-5′,5′-dimethyl-8-benzyl-azaspiro[bicyclo[3.2.1]octane-3,2′-[1,3]dioxolane]-6-ol, 424

Compound 424 isolated as a solid foam (72.9%), was synthesized from compound 224 by following the procedure described in Example 52. ES MS: [M+H]⁺ 667.7 (observed), 666.8 (calcd.).

### Example 54

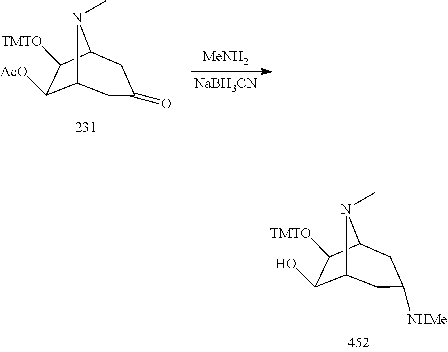

### rel-(1R,5S,6S,7R)-7-[(Tris(4-methoxyphenyl)methoxy]-8-methyl-3-(methylamino)-aza[bicyclo[3.2.1]octane-6-ol, 452

The reaction was carried out by modification of the method disclosed in Lewin, A. H.; Sun, G.; Fudala, L.; Navarro, H.; Zhou, L.-M.; Popick, P.; Faynsteyn, A.; Skolnick, P. Molecular Features Associated with Polyamine Modulation of NMDA Receptors. J. Medicinal Chem. 1998, 41 (6), 988-995. A mixture of 40% aq. methylamine (1.94 g, 25 mmol) and MeOH (40 mL) was neutralized with conc. aq. HCl to pH 6.5-7. Compound 231 (3.27 g, 6.0 mmol) was added to this mixture followed by NaBH₃CN (1.2 g). The solution was stirred at room temperature for 3 days and evaporated. The residue was distributed between H₂O (10 mL) and DCM (100 mL). The organic phase was washed with 1M NaH₂PO₄ (4×50 mL). The aqueous phase was basified with solid NaOH and extracted with DCM (3×50 mL). After drying over Na₂SO₄, the combined DCM solutions were evaporated and dried under vacuum. The crude product (2.65 g, 85%) was used in the next step without further purification.

### Example 55

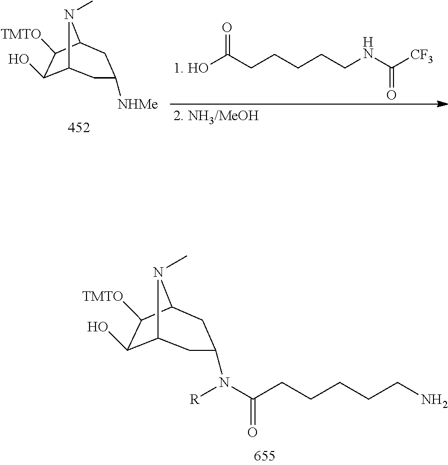

### rel-(1R,5S,6S,7R)-7-[(Tris(4-methoxyphenyl)methoxy]-8-methyl-3-[6-(trifluoroacetylamino)hexanmido]-aza[bicyclo[3.2.1]octane-6-ol, 655

N-trifluoroacetyl-6-aminohexanoic acid was converted to its N-oxysuccinimidyl ester, 2,5-dioxo-1-pyrrolidinyl 6-[(2,2,2-trifluoroacetyl) amino]hexanoate, as disclosed in WO2008102606. This (1.78 g, 5.5 mmol) was dissolved in pyridine (25 mL), and the solution obtained was added to the crude compound 452 (2.59 g, 5 mmol). The mixture was stirred overnight at room temperature and was quenched by adding TEA (1.02 g, 10 mmol) and water (1 mL). After stirring for 2 h, 10% methanolic ammonia (10 mL) was added, and stirring was continued for 3 h. The solvents were evaporated, the residue was dissolved in ethyl acetate (100 mL). The solution obtained was washed 0.5 M aq NaOH (2×20 mL) and brine (2×50 mL). After drying over Na₂SO₄, the organic phase was evaporated. The material obtained was purified by column chromatography on silica gel eluting with a linear gradient from DCM to DCM: MeOH: 30% aq. ammonia (80:15:5). Upon evaporation of collected fractions, the desired product was obtained as a white solid foam (2.60 g, 82.4%). ES MS: [M+H]⁺ 633.3 (observed), 632.8 (calcd.).

### Example 56

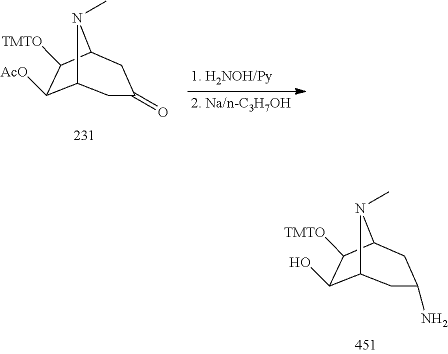

### rel-(1R,5S,6S,7R)-7-[(Tris(4-methoxyphenyl)methoxy]-8-methyl-3-amino-aza[bicyclo[3.2.1]octane-6-ol, 451

Compound 451 was prepared by modification of the method disclosed in Lewin, A. H.; Sun, G.; Fudala, L.; Navarro, H.; Zhou, L.-M.; Popick, P.; Faynsteyn, A.; Skolnick, P. Molecular Features Associated with Polyamine Modulation of NMDA Receptors. J. Medicinal Chem. 1998, 41 (6), 988-995.

A mixture of compound 231 (7.5 g, 5 mmol), H₂NOH—HCl (5.44 g, 78 mmol), and pyridine (10 mL) in EtOH (110 mL) was stirred at room temperature for 24 h. The mixture was concentrated, and the residue was distributed between 2.5 M aq. NaOH (40 mL) and ethyl acetate (100 mL). The aqueous phase was extracted twice with ethyl acetate (50 mL), and the combined organic phases were washed with brine (50 mL) and dried over K₂CO₃. The solvent was evaporated, and the residue was used in the next step without purification.

The product of the previous step was dissolved in anhydrous n-propanol (100 mL). To this, sodium wirings (2.3 g, 100 mmol) were added over 20 min, and the mixture was refluxed for 1.5 h. After cooling, water (5 mL) was added, and the solvent was evaporated. The residue was distributed between water (50 mL) and ethyl acetate (100 mL). The aqueous phase was extracted twice with ethyl acetate (50 mL), and the combined organic phases were washed with brine (50 mL) and dried over K₂CO₃. The crude material obtained was purified by column chromatography on silica gel eluting with a linear gradient from DCM to DCM: MeOH: 30% aq. ammonia (80:15:5). Upon evaporation of collected fractions, the desired product was obtained as a white solid foam (1.48 g, 58.7%). ES MS: [M+H]⁺ 506.5 (observed), 505.6 (calcd.).

### Example 57

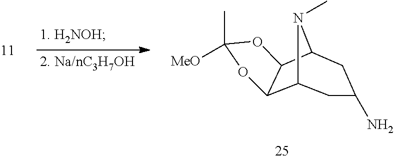 [SMILES: COC1(C)OC2C(O1)C1CC(N)CC2N1C]

### 2,9-Dimethyl-2-methoxy-6-amino-hexahydro-6H-cyclohepta-1,3-dioxol-4,8-imin, 25

Compound 25 isolated as a hygroscopic solid (46.7%), was synthesized from compound 11 by following the procedure described in Example 56. ES MS: [M+H]⁺ 230.1 (observed), 229.3 (calcd.).

### Example 58

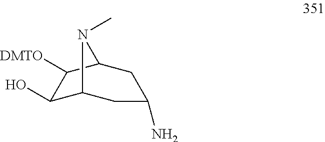

### rel-(1R,5S,6S,7R)-7-[(Bis(4-methoxyphenyl)(phenyl)methoxy]-8-methyl-3-amino-aza[bicyclo[3.2.1]octane-6-ol, 351

Compound 351 isolated as a white solid foam (60.6%), was synthesized from compound 131 by following the procedure described in Example 56. ES MS: [M+H]⁺ 476.1 (observed), 475.6 (calcd.).

### Example 59

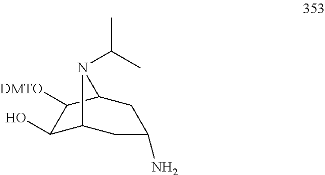

### rel-(1R,5S,6S,7R)-7-[(Bis(4-methoxyphenyl)(phenyl)methoxy]-8-isopropyl-3-amino-aza[bicyclo[3.2.1]octane-6-ol, 353

Compound 353 isolated as a white solid foam (64.4%), was synthesized from compound 133 by following the procedure described in Example 56. ES MS: [M+H]⁺ 504.3 (observed), 503.7 (calcd.).

### Example 60

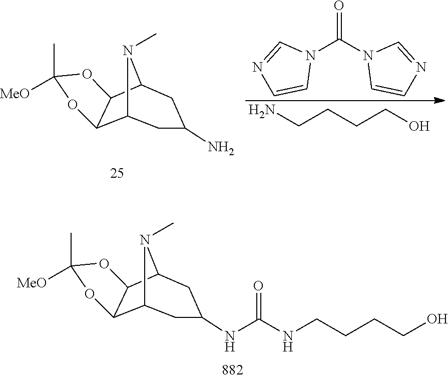

### N-(2,9-Dimethyl-2-methoxy-6-amino-hexahydro-6H-cyclohepta-1,3-dioxol-4,8-imin-6-yl)-N′-(4-hydroxybutyl) urea, 882

Compound 25 (457 mg, 2.0 mmol) in pyridine (10 mL) was added dropwise to N,N′-carbonyldiimidazole (324 mg, 2.0 mmol) in pyridine (10 mL) under stirring and cooling in an ice bath. The mixture was stirred overnight at room temperature. Next, 4-aminobutanol (267 mg, 3.0 mmol) in acetonitrile (4 mL) was added, and the mixture was stirred for 24 h. Solvents were evaporated, the residue was distributed between dichloromethane (100 mL) and 1M NaH₂PO₄ (20 mL). The organic phase was washed with 1M NaH₂PO₄ (2×50 mL) and then with brine (30 mL). After drying over Na₂SO₄, the organic phase was evaporated. and dried under vacuum. The product was isolated by column purification on silica gel using a linear gradient from DCM to 15% MeOH in DCM. Evaporation of collected fractions and drying in vacuo gave the desired product as a solid foam (620 mg, 90.3%). ES MS: [M+H]⁺ 345.0 (observed), 344.4 (calcd.).

### Example 61

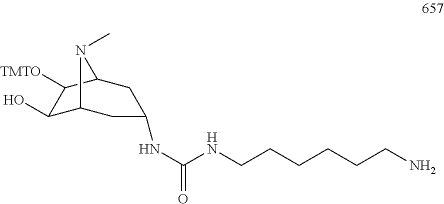

### rel-(1R,5S,6S,7R)—N-[7-[(Tris(4-methoxyphenyl)methoxy]-8-methyl-6-hydroxy-aza[bicyclo[3.2.1]octane-6-yl]-N′-(6-aminohexyl) urea, 657

Compound 657 isolated as a white solid foam (92.1%), was synthesized from compound 451 by following the procedure described in Example 60. ES MS: [M+H]⁺ 648.5 (observed), 647.8 (calcd.).

### Example 62

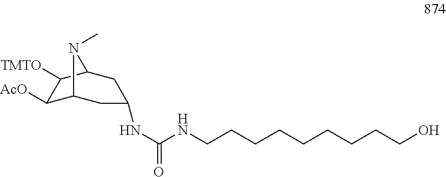

### rel-(1R,5S,6S,7R)—N-[7-[(Tris(4-methoxyphenyl)methoxy]-8-methyl-6-acetoxy-aza[bicyclo[3.2.1]octane-6-yl]-N′-(9-hydroxynonyl) urea, 874

Fluorenylmethyloxycarbonyl chloride (543 mg, 2.1 mmol) in acetonitrile was added dropwise to compound 451 (1009 mg, 2.0 mmol) in acetonitrile (10 mL) under stirring in an ice bath. The mixture was stirred for 1 h, after which time pyridine (5 mL) and acetic anhydride (306 mg, 3 mmol) were added. The mixture was left overnight, and then 10% aq. diethylamine (2.9 g, 4 mmol) was added. Upon stirring for 1 h, the mixture was evaporated, and the residue was dissolved in ethyl acetate (50 mL) and washed with 5% aq. NaHCO₃ (2×50 mL) and with brine (50 mL). Organic phase was dried over Na₂SO₄ and evaporated.

The residue was dissolved in pyridine (10 mL) and was added dropwise to N,N′-carbonyldiimidazole (359 mg, 2.1 mmol) in pyridine (10 mL) under stirring and cooling in an ice bath. The mixture was stirred overnight at room temperature. Next, 9-aminononanol (477 mg, 3.0 mmol) in acetonitrile (4 mL) was added, and the mixture was stirred for 24 h. Solvents were evaporated, the residue was distributed between dichloromethane (100 mL) and 1M NaH₂PO₄ (20 mL). The organic phase was washed with 1M NaH₂PO₄ (2×50 mL) and then with brine (30 mL). After drying over Na₂SO₄, the organic phase was evaporated. and dried under vacuum. The product was isolated by column purification on silica gel using a linear gradient from DCM to 15% MeOH in DCM. Evaporation of collected fractions and drying in vacuo gave the desired product as a solid foam (1121 mg, 76.6%). ES MS: [M+H]⁺ 733.4 (observed), 732.9 (calcd.).

### Example 63

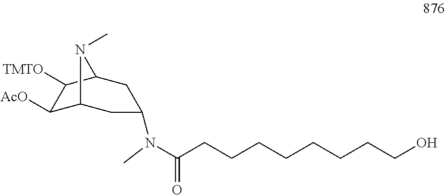

### rel-(1R,5S,6S,7R)—N-methyl-N-[7-[(Tris(4-methoxyphenyl)methoxy]-8-methyl-6-acetoxy-aza[bicyclo[3.2.1]octane-6-yl]-(9-hydroxy) nonanamide, 876

Fluorenylmethyloxycarbonyl chloride (310 mg, 1.2 mmol) in acetonitrile was added dropwise to compound 452 (561 mg, 1.0 mmol) and DIPEA (139 mg, 1.1 mmol) in acetonitrile (10 mL) under stirring in an ice bath. The mixture was stirred for 6 h, after which time pyridine (5 mL) and acetic anhydride (204 mg, 2 mmol) were added. The mixture was left overnight, and then 10% aq. diethylamine (2.9 g, 4 mmol) was added. Upon stirring for 1 h, the mixture was evaporated, and the residue was dissolved in ethyl acetate (50 mL) and washed with 5% aq. NaHCO₃ (2×50 mL) and with brine (50 mL). Organic phase was dried over Na₂SO₄ and evaporated. The residue was dried on an oil pump.

Disuccinimidyl carbonate (384 mg, 1.5 mmol) was reacted with 9-hydroxynonanoic acid (261 mg, 1.5 mmol) in pyridine (5 mL) overnight. The resulting solution was added to the dried product of the previous step, and the mixture was allowed to react overnight. Solvents were evaporated, the residue was distributed between ethyl acetate (100 mL) and 5% aq. NaHCO₃ (20 mL). The organic phase was washed with 5% aq. NaHCO₃ (2×20 mL) and then with brine (30 mL). After drying over Na₂SO₄, the organic phase was evaporated. and dried under vacuum. The product was isolated by column purification on silica gel using a linear gradient from DCM to 15% MeOH in DCM. Evaporation of collected fractions and drying in vacuo gave the desired product as a solid foam (510 mg, 71.2%). ES MS: [M+H]⁺ 718.7 (observed), 717.9 (calcd.).

### Example 64

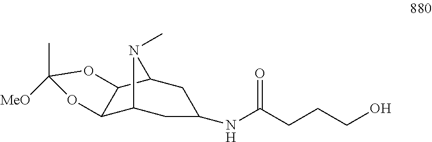 [SMILES: COC1(C)OC2C(O1)C1CC(NC(=O)CCCO)CC2N1C]

### N-(2,9-Dimethyl-2-methoxy-6-amino-hexahydro-6H-cyclohepta-1,3-dioxol-4,8-imin-6-yl)-(4-hydroxy) butanamide, 880

Disuccinimidyl carbonate (563 mg, 2.2 mmol) was reacted with 4-hydroxybutanoic acid (348 mg, 1.5 mmol) in pyridine (5 mL) overnight. The resulting solution was added to compound 25 (458 mg, 2.0 mmol), and the mixture was allowed to react overnight. Solvents were evaporated, the residue was distributed between ethyl acetate (100 mL) and 5% aq. NaHCO₃ (20 mL). The organic phase was washed with 5% aq. NaHCO₃ (2×20 mL) and then with brine (30 mL). After drying over Na₂SO₄, the organic phase was evaporated. and dried under vacuum. The product was isolated by column purification on silica gel using a linear gradient from DCM to 25% MeOH in DCM. Evaporation of collected fractions and drying in vacuo gave the desired product as a solid foam (560 mg, 89.1%). ES MS: [M+H]⁺ 316.2 (observed), 315.4 (calcd.).

### Example 65

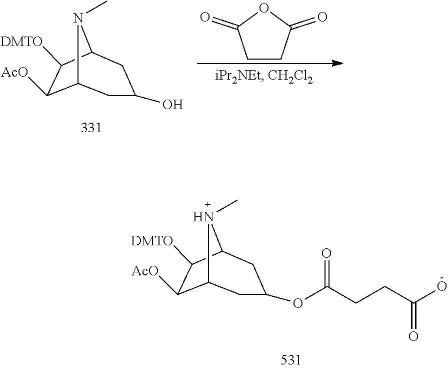

### (1S,5R,6R,7S)-[[6-Acetoxy-7-(bis(4-methoxyphenyl)(phenyl)methoxy)-3-hydroxy-8-methyl-8-azabicyclo[3.2.1]octan-3-yl]oxy]-4-oxobutanoic acid, 531

To a solution of (1S,5R,6R,7S)-7-(bis(4-methoxyphenyl)(phenyl)methoxy)-3-hydroxy-8-methyl-8-azabicyclo[3.2.1]octan-6-yl acetate 331 (30.02 g, 58 mmol) in DCM (300 mL) was added iPr₂NEt (22.5 g, 174 mmol), dimethyl amino pyridine (360 mg, 2.9 mmol) and succinic anhydride (11.7 g, 116.5 mmol). The mixture was stirred at room temperature for 3 hours and quenched with water: triethylamine (9:1)(100 mL). The organic layer was diluted with DCM (300 mL) and was extracted with water (500 mL). The organic extract was separated and evaporated. The crude residue was treated with a solution of water-triethylamine (19:1)(300 mL) and extracted with a solution of ethyl acetate:hexane (80:20) (500 mL). The organic extract was discarded and the water layer was acidified with 5% aq. citric acid until pH 6.5-7.0 was reached. The product separated as an oil was extracted with DCM (3×500 mL), extracts were dried over sodium sulfate, evaporated, and dried on an oil pump. The product 531 was obtained as white foam (34.03 g, 95.0%).

¹H-NMR (500 MHz, CDCl₃): δ 7.4 (m, 2H); 7.3 (m, 8H); 6.8 (m, 4H); 5.3 (m, 1H); 4.6 (m, 1H); 4.4 (d, 1H); 3.8 (s, 6H); 3.6 (s, 1H); 2.9 (s, 3H); 2.6 (m, 1H); 2.5 (m, 4H); 2.2 (s, 3H); 2.1 (m, 2H); 1.9 (m, 1H); 1.5 (m, 1H). ¹³C-NMR (100 MHz, DMSO-d₆): δ 14.05, 20.72, 20.96, 28.56, 28.7, 54.87, 55.08, 59.71, 113.33, 126.94, 127.89, 129.77, 129.86, 158.31, 169.65, 171.36, 173.19. ES MS: [M+H]⁺ 618.3, [2M+H]⁺ 1235.5 (obtained); 617.3 (calcd.).

### Example 66

### (rel-(1R,3-endo,5S,6S,7R)-7-(bis(4-methoxyphenyl)(phenyl)methoxy)-3-pivaloyloxy-8-(i-propyl)-8-azabicyclo[3.2.1]octane-6-yl) hemisuccinate, 503. (FIG. 2)

A solution of compound 303 (2.17 mg, 3.69 mmol) and succinic anhydride (1.84 g, 18.44 mmol) in pyridine (67.1 mL) was stirred for 3 days at 35° C. The mixture was quenched with water (0.66 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 503 (2.20 g, 86.7%) as a white solid foam. ES MS: [M−1]⁻ 687.8.

### Example 67

### (rel-(1R,3-endo,5S,6S,7R)-7-(bis(4-methoxyphenyl)(phenyl)methoxy)-3-isobutyryloxy-8-benzyl-8-azabicyclo[3.2.1]octane-6-yl) hemisuccinate, 505. (FIG. 2)

A solution of compound 305 (3.07 mg, 4.94 mmol) and succinic anhydride (2.47 g, 24.68 mmol) in pyridine (89.8 mL) was stirred for 3 days at 35° C. The mixture was quenched with water (0.89 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 505 (3.03 g, 85.0%) as a white solid foam. ES MS: [M−1]⁻ 721.9.

### Example 68

### (rel-(1R,3-endo,5S,6S,7R)-7-(tris(4-methoxyphenyl)methoxy)-3-acetoxy-8-methyl-8-azabicyclo[3.2.1]octane-6-yl)hemisuccinate, 601. (FIG. 2)

A solution of compound 401 (2.82 mg, 5.15 mmol) and succinic anhydride (2.57 g, 25.73 mmol) in pyridine (93.6 mL) was stirred for 3 days at 35° C. The mixture was quenched with water (0.93 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 601 (3.06 g, 91.7%) as a white solid foam. ES MS: [M−1]⁻ 647.7.

### Example 69

### (rel-(1R,3-endo,5S,6S,7R)-7-(tris(4-methoxyphenyl)methoxy)-3-pivaloyloxy-8-(i-propyl)-8-azabicyclo[3.2.1]octane-6-yl) hemisuccinate, 603. (FIG. 2)

A solution of compound 403 (3.17 mg, 5.12 mmol) and succinic anhydride (2.56 g, 25.61 mmol) in pyridine (93.2 mL) was stirred for 3 days at 35° C. The mixture was quenched with water (0.92 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 603 (3.04 g, 82.7%) as a white solid foam. ES MS: [M−1]⁻ 717.9.

### Example 70

### (rel-(1R,3-endo,5S,6S,7R)-7-(tris(4-methoxyphenyl)methoxy)-3-isobutyryloxy-8-methyl-8-azabicyclo[3.2.1]octane-6-yl) hemisuccinate, 605. (FIG. 2)

A solution of compound 405 (3.07 mg, 4.70 mmol) and succinic anhydride (2.35 g, 23.51 mmol) in pyridine (85.6 mL) was stirred for 3 days at 35° C. The mixture was quenched with water (0.85 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 605 (3.11 g, 88.1%) as a white solid foam. ES MS: [M−1]⁻ 751.9.

### Example 71

### (rel-(1R,3-endo,5S,6S,7R)-7-(tris(4-methoxyphenyl)methoxy)-3-[(t-butyl)dimethylsilyloxy]-8-methyl-8-azabicyclo[3.2.1]octane-6-yl) hemisuccinate, 616. (FIG. 2)

A solution of compound 416 (2.67 mg, 4.30 mmol) and succinic anhydride (2.15 g, 21.51 mmol) in pyridine (78.3 mL) was stirred for 3 days at 35° C. The mixture was quenched with water (0.77 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 616 (2.72 g, 87.9%) as a white solid foam. ES MS: [M−1]⁻ 720.0.

### Example 72

### (rel-(1R,3-endo,5S,6S,7R)-7-(tris(4-methoxyphenyl)methoxy)-3-[(t-butyl) diphenylsilyloxy]-8-methyl-8-azabicyclo[3.2.1]octane-6-yl) hemisuccinate, 618. (FIG. 2)

A solution of compound 418 (3.13 mg, 4.21 mmol) and succinic anhydride (2.11 g, 21.05 mmol) in pyridine (76.6 mL) was stirred for 3 days at 35° C. The mixture was quenched with water (0.76 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 618 (2.95 g, 82.9%) as a white solid foam. ES MS: [M−1]⁻ 844.1.

### Example 73

### (rel-(1R,3-endo,5S,6S,7R)-7-(tris(4-methoxyphenyl)methoxy)-8-methyl-8-azaspiro[bicyclo[3.2.1]octane-3,2′-[1,3]dioxolane]-6-yl) hemisuccinate, 621. (FIG. 4)

A solution of compound 421 (2.19 mg, 4.01 mmol) and succinic anhydride (2.00 g, 20.03 mmol) in pyridine (72.9 mL) was stirred for 3 days at 35° C. The mixture was quenched with water (0.72 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 621 (2.28 g, 87.8%) as a white solid foam. ES MS: [M−1]⁻ 647.7.

### Example 74

### (rel-(1R,3-endo,5S,6S,7R)-7-(tris(4-methoxyphenyl)methoxy)-8-methyl-8-azaspiro[bicyclo[3.2.1]octane-3,2′-[1,3]dioxane]-6-yl) hemisuccinate, 622. (FIG. 4)

A solution of compound 422 (3.12 mg, 5.30 mmol) and succinic anhydride (2.65 g, 26.49 mmol) in pyridine (96.4 mL) was stirred for 3 days at 35° C. The mixture was quenched with water (0.95 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 622 (3.20 g, 87.6%) as a white solid foam. ES MS: [M−1]⁻ 689.8.

### Example 75

### (rel-(1R,3-endo,5S,6S,7R)-7-(tris(4-methoxyphenyl)methoxy)-8-(i-propyl)-8-azaspiro[bicyclo[3.2.1]octane-3,2′-[1,3]dioxolane]-6-yl) hemisuccinate, 623. (FIG. 4)

A solution of compound 423 (1.89 mg, 3.28 mmol) and succinic anhydride (1.64 g, 16.42 mmol) in pyridine (59.7 mL) was stirred for 3 days at 35° C. The mixture was quenched with water (0.59 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 623 (1.99 g, 89.8%) as a white solid foam. ES MS: [M−1]⁻ 675.8.

### Example 76

### (rel-(1R,3-endo,5S,6S,7R)-7-(tris(4-methoxyphenyl)methoxy)-8-benzyl-8-azaspiro[bicyclo[3.2.1]octane-3,2′-[1,3]dioxane]-6-yl) hemisuccinate, 624. (FIG. 4)

A solution of compound 424 (4.16 mg, 6.24 mmol) and succinic anhydride (3.12 g, 31.22 mmol) in pyridine (113.6 mL) was stirred for 3 days at 35° C. The mixture was quenched with water (1.12 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 624 (4.16 g, 86.9%) as a white solid foam. ES MS: [M−1]⁻ 765.9.

### Example 77

### (rel-(1R,3-endo,5S,6S,7R)-7-(bis(4-methoxyphenyl)(phenyl)methoxy)-6-acetoxy-8-methyl-8-azabicyclo[3.2.1]octane-3-yl) hemisuccinate, 531. (FIG. 6)

A solution of compound 331 (4.20 mg, 8.12 mmol) and succinic anhydride (4.06 g, 40.61 mmol) in pyridine (147.8 mL) was stirred for 3 days at 35° C. The mixture was quenched with water (1.46 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 531 (4.38 g, 87.4%) as a white solid foam. ES MS: [M−1]⁻ 617.7.

### Example 78

### (rel-(1R,3-endo,5S,6S,7R)-7-(bis(4-methoxyphenyl)(phenyl)methoxy)-6-acetoxy-8-ethyl-8-azabicyclo[3.2.1]octane-3-yl) hemisuccinate, 532. (FIG. 6)

A solution of compound 332 (2.39 mg, 4.49 mmol) and succinic anhydride (2.25 g, 22.46 mmol) in pyridine (81.7 mL) was stirred for 3 days at 35° C. The mixture was quenched with water (0.81 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 532 (2.37 g, 83.4%) as a white solid foam. ES MS: [M−1]⁻ 631.7.

### Example 79

### (rel-(1R,3-endo,5S,6S,7R)-7-(bis(4-methoxyphenyl)(phenyl)methoxy)-6-acetoxy-8-(i-propyl)-8-azabicyclo[3.2.1]octane-3-yl) hemisuccinate, 533. (FIG. 6)

A solution of compound 333 (2.71 mg, 4.96 mmol) and succinic anhydride (2.48 g, 24.81 mmol) in pyridine (90.3 mL) was stirred for 3 days at 35° C. The mixture was quenched with water (0.89 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 533 (2.80 g, 87.5%) as a white solid foam. ES MS: [M−1]⁻ 645.8.

### Example 80

### (rel-(1R,3-endo,5S,6S,7R)-7-(bis(4-methoxyphenyl)(phenyl)methoxy)-6-acetoxy-8-benzyl-8-azabicyclo[3.2.1]octane-3-yl) hemisuccinate, 534. (FIG. 6)

A solution of compound 334 (2.93 mg, 4.93 mmol) and succinic anhydride (2.47 g, 24.65 mmol) in pyridine (89.7 mL) was stirred for 3 days at 35° C. The mixture was quenched with water (0.89 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 534 (2.95 g, 86.4%) as a white solid foam. ES MS: [M−1]⁻ 693.8.

### Example 81

### (rel-(1R,3-endo,5S,6S,7R)-7-(bis(4-methoxyphenyl)(phenyl)methoxy)-6-acetoxy-8-methyl-8-azabicyclo[3.2.1]octane-3-yl) diglycolate, 535. (FIG. 6)

A solution of compound 331 (3.14 mg, 6.06 mmol) and succinic anhydride (3.03 g, 30.29 mmol) in pyridine (110.2 mL) was stirred for 3 days at 35° C. The mixture was quenched with water (1.09 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 535 (3.19 g, 83.0%) as a white solid foam. ES MS: [M−1]⁻ 633.7.

### Example 82

### (rel-(1R,3-endo,5S,6S,7R)-7-(bis(4-methoxyphenyl)(phenyl)methoxy)-6-acetoxy-2,4,8-trimethyl-8-azabicyclo[3.2.1]octane-3-yl) hemisuccinate, 537. (FIG. 6)

A solution of compound 337 (3.20 mg, 5.86 mmol) and succinic anhydride (2.93 g, 29.28 mmol) in pyridine (106.6 mL) was stirred for 3 days at 35° C. The mixture was quenched with water (1.05 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 537 (3.48 g, 92.0%) as a white solid foam. ES MS: [M−1]⁻ 645.8.

### Example 83

### (rel-(1R,3-endo,5S,6S,7R)-7-(bis(4-methoxyphenyl)(phenyl)methoxy)-6-acetoxy-2,2,4,4,8-pentamethyl-8-azabicyclo[3.2.1]octane-3-yl) hemisuccinate, 538. (FIG. 6)

A solution of compound 338 (4.00 mg, 6.97 mmol) and succinic anhydride (3.49 g, 34.83 mmol) in pyridine (126.8 mL) was stirred for 3 days at 35° C. The mixture was quenched with water (1.25 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 538 (4.27 g, 91.0%) as a white solid foam. ES MS: [M−1]⁻ 673.8.

### Example 84

### (rel-(1R,3-endo,5S,6S,7R)-7-(tris(4-methoxyphenyl)methoxy)-6-acetoxy-8-methyl-8-azabicyclo[3.2.1]octane-3-yl) hemisuccinate, 631. (FIG. 6)

A solution of compound 431 (3.49 mg, 6.36 mmol) and succinic anhydride (3.18 g, 31.78 mmol) in pyridine (115.7 mL) was stirred for 3 days at 35° C. The mixture was quenched with water (1.14 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 631 (3.60 g, 87.5%) as a white solid foam. ES MS: [M−1]⁻ 647.7.

### Example 85

### (rel-(1R,3-endo,5S,6S,7R)-7-(tris(4-methoxyphenyl)methoxy)-6-acetoxy-8-ethyl-8-azabicyclo[3.2.1]octane-3-yl) hemisuccinate, 632. (FIG. 6)

A solution of compound 432 (2.60 mg, 4.63 mmol) and succinic anhydride (2.32 g, 23.14 mmol) in pyridine (84.2 mL) was stirred for 3 days at 35° C. The mixture was quenched with water (0.83 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 632 (2.69 g, 88.0%) as a white solid foam. ES MS: [M−1]⁻ 661.8.

### Example 86

### (rel-(1R,3-endo,5S,6S,7R)-7-(tris(4-methoxyphenyl)methoxy)-6-acetoxy-8-(i-propyl)-8-azabicyclo[3.2.1]octane-3-yl) hemisuccinate, 633. (FIG. 6)

A solution of compound 433 (2.80 mg, 4.86 mmol) and succinic anhydride (2.43 g, 24.28 mmol) in pyridine (88.4 mL) was stirred for 3 days at 35° C. The mixture was quenched with water (0.87 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 633 (2.94 g, 89.7%) as a white solid foam. ES MS: [M−1]⁻ 675.8.

### Example 87

### (rel-(1R,3-endo,5S,6S,7R)-7-(tris(4-methoxyphenyl)methoxy)-6-acetoxy-8-benzyl-8-azabicyclo[3.2.1]octane-3-yl) hemisuccinate, 634. (FIG. 6)

A solution of compound 434 (2.50 mg, 4.00 mmol) and succinic anhydride (2.00 g, 19.98 mmol) in pyridine (72.7 mL) was stirred for 3 days at 35° C. The mixture was quenched with water (0.72 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 634 (2.64 g, 91.2%) as a white solid foam. ES MS: [M−1]⁻ 723.8.

### Example 88

### (rel-(1R,3-endo,5S,6S,7R)-7-(tris(4-methoxyphenyl)methoxy)-6-acetoxy-8-methyl-8-azabicyclo[3.2.1]octane-3-yl) [2-[4-(carboxymethoxy) phenoxy]acetate], 636. (FIG. 6)

A solution of compound 431 (2.83 mg, 5.17 mmol) and succinic anhydride (2.59 g, 25.83 mmol) in pyridine (94.0 mL) was stirred for 3 days at 35° C. The mixture was quenched with water (0.93 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 636 (3.36 g, 86.0%) as a white solid foam. ES MS: [M−1]⁻ 755.8.

### Example 89

### (rel-(1R,3-endo,5S,6S,7R)-7-(tris(4-methoxyphenyl)methoxy)-6-acetoxy-2,4,8-trimethyl-8-azabicyclo[3.2.1]octane-3-yl) hemisuccinate, 637. (FIG. 6)

A solution of compound 437 (3.00 mg, 5.20 mmol) and succinic anhydride (2.60 g, 25.98 mmol) in pyridine (94.6 mL) was stirred for 3 days at 35° C. The mixture was quenched with water (0.94 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 637 (3.04 g, 86.7%) as a white solid foam. ES MS: [M−1]⁻ 675.8.

### Example 90

### (rel-(1R,3-endo,5S,6S,7R)-7-(tris(4-methoxyphenyl)methoxy)-6-acetoxy-2,2,4,4,8-pentamethyl-8-azabicyclo[3.2.1]octane-3-yl) hemisuccinate, 638. (FIG. 6)

A solution of compound 438 (3.15 mg, 5.22 mmol) and succinic anhydride (2.61 g, 26.08 mmol) in pyridine (94.9 mL) was stirred for 3 days at 35° C. The mixture was quenched with water (0.94 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 638 (3.19 g, 86.9%) as a white solid foam. ES MS: [M−1]⁻ 703.8.

### Example 91

### (rel-(1R,3-endo,5S,6S,7R)-7-(tris(4-methoxyphenyl)methoxy)-3-methoxy-8,8-dimethyl-8-azoniabicyclo[3.2.1]octane-6-yl) hemisuccinate, 671. (FIG. 3)

A solution of compound 411 (2.83 mg, 4.77 mmol) and succinic anhydride (2.39 g, 23.86 mmol) in pyridine (86.8 mL) was stirred for 3 days at 35° C. The mixture was quenched with water (0.86 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 671 (3.23 g, 85.2%) as a white solid foam. ES MS: [M−1]⁻ 794.0.

### Example 92

### (rel-(1R,3-endo,5S,6S,7R)-7-(tris(4-methoxyphenyl)methoxy)-3-benzyloxy-8-methyl-8-benzyl-8-azoniabicyclo[3.2.1]octane-6-yl) hemisuccinate, 674. (FIG. 3)

A solution of compound 414 (3.55 mg, 4.77 mmol) and succinic anhydride (2.38 g, 23.83 mmol) in pyridine (86.7 mL) was stirred for 3 days at 35° C. The mixture was quenched with water (0.86 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 674 (3.99 g, 88.4%) as a white solid foam. ES MS: [M−1]⁻ 946.2.

### Example 93

### (rel-(1R,3-endo,5S,6S,7R)-7-(tris(4-methoxyphenyl)methoxy)-3-methoxy-8-methyl-8-benzyl-8-azoniabicyclo[3.2.1]octane-6-yl) hemisuccinate, 675. (FIG. 3)

A solution of compound 415 (3.39 mg, 5.06 mmol) and succinic anhydride (2.53 g, 25.31 mmol) in pyridine (92.1 mL) was stirred for 3 days at 35° C. The mixture was quenched with water (0.91 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 675 (3.59 g, 81.4%) as a white solid foam. ES MS: [M−1]⁻ 870.1.

### Example 94

### N-[(rel-(1R,3-endo,5S,6S,7R)-7-(bis(4-methoxyphenyl)(phenyl)methoxy)-6-acetoxy-8-methyl-8-azabicyclo[3.2.1]octane-3-yl)]succinamic acid, 551. (FIG. 5)

A solution of compound 351 (2.41 mg, 4.66 mmol) and succinic anhydride (2.33 g, 23.29 mmol) in pyridine (84.8 mL) was stirred for 3 days at 35° C. The mixture was quenched with water (0.84 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 551 (2.45 g, 85.2%) as a white solid foam. ES MS: [M−1]⁻ 616.7.

### Example 95

### N-[(rel-(1R,3-endo,5S,6S,7R)-7-(bis(4-methoxyphenyl)(phenyl)methoxy)-6-acetoxy-8-(i-propyl)-8-azabicyclo[3.2.1]octane-3-yl)]succinamic acid, 553. (FIG. 5)

A solution of compound 353 (2.00 mg, 3.67 mmol) and succinic anhydride (1.84 g, 18.35 mmol) in pyridine (66.8 mL) was stirred for 3 days at 35° C. The mixture was quenched with water (0.66 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 553 (2.01 g, 84.9%) as a white solid foam. ES MS: [M−1]⁻ 644.8.

### Example 96

### N-[(rel-(1R,3-endo,5S,6S,7R)-7-(tris(4-methoxyphenyl)methoxy)-6-acetoxy-8-methyl-8-azabicyclo[3.2.1]octane-3-yl)]succinamic acid, 651. (FIG. 5)

A solution of compound 451 (1.82 mg, 3.33 mmol) and succinic anhydride (1.66 g, 16.64 mmol) in pyridine (60.5 mL) was stirred for 3 days at 35° C. The mixture was quenched with water (0.60 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA (95:5) to give compound 651 (1.80 g, 83.5%) as a white solid foam. ES MS: [M−1]⁻ 646.

### Example 97

### N-methyl-N-[(rel-(1R,3-endo,5S,6S,7R)-7-(tris(4-methoxyphenyl)methoxy)-6-acetoxy-8-methyl-8-azabicyclo[3.2.1]octane-3-yl)]succinamic acid, 652

A solution of compound 452 (2.23 mg, 3.97 mmol) and succinic anhydride (1.99 g, 19.87 mmol) in pyridine (72.3 mL) was stirred for 3 days at 35° C. The mixture was quenched with water (0.72 mL) and triethylamine (5.0 mL) for 2 h at room temperature and evaporated in vacuo. The residue was dissolved in DCM (100 mL) and washed with 0.5 M aq. triethylammonium acetate. The organic phase was dried over Na₂SO₄, filtered, and evaporated in vacuo. The crude material was purified on a silica gel column using a linear gradient of MeOH (0 to 10% MeOH) in DCM-TEA.

### Example 98

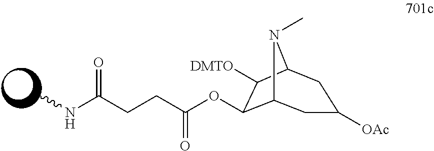

### (rel-(1R,3-endo,5S,6S,7R)-7-[(Bis(4-methoxyphenyl)(phenyl)methoxy]-3-acetoxy-8-methyl-aza[bicyclo[3.2.1]octane-6-yl) hemisuccinate, covalently bound to aminopropyl CPG500, 701c

TBTU (0.26 g, 0.81 mmol) was added to a solution of compound 501 (0.48 g, 0.77 mmol) and N,N-diisopropylethylamine (0.239 g, 1.85 mmol) in a mixture of anhydrous pyridine (1.25 mL) and acetonitrile (8 mL). The mixture was stirred for 15 min and transferred to a suspension of aminopropyl CPG500 (10 g) in anhydrous acetonitrile (45 mL), and the resulting suspension was shaken for 4 h. The suspension was then charged with N-methylimidazole (2.0 mL) and acetic anhydride (2.0 mL) and was shaken again for 45 min. The solid support was filtered off, washed on the filter with acetonitrile (5×50 mL) and dried in vacuo. The loading of the finished solid support 701c (69.0 μmol/g) was determined, as applicable, by the standard di- or trimethoxytrityl assay as disclosed in Guzaev, A. P. and Pon, R. T. Curr. Protoc. Nucleic Acid Chem. 2013, 52, pp. 3.2.1-3.2.23.

### Example 99

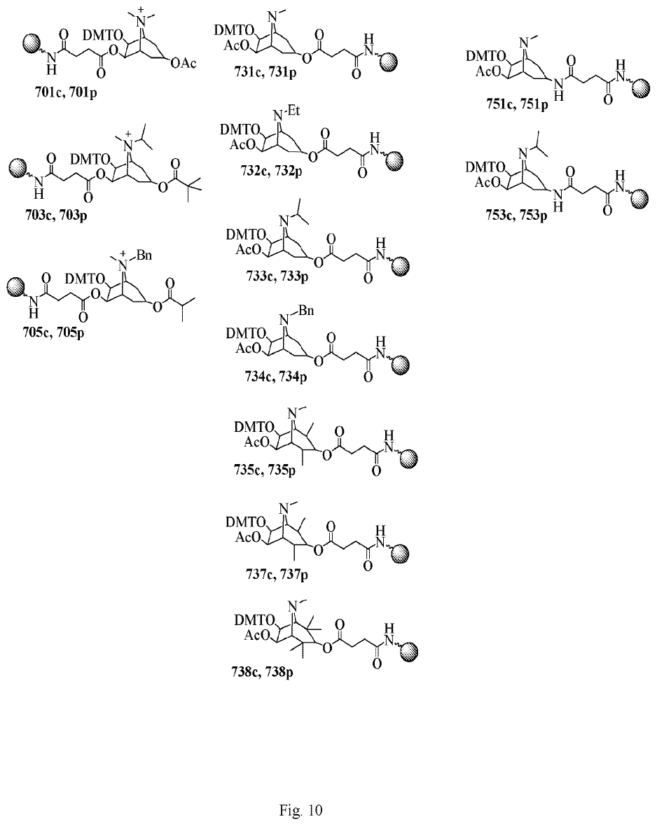

### (rel-(1R,3-endo,5S,6S,7R)-7-(bis(4-methoxyphenyl)(phenyl)methoxy)-3-pivaloyloxy-8-(i-propyl)-8-azabicyclo[3.2.1]octane-6-yl) hemisuccinate, covalently bound to aminopropyl CPG1500, Solid support 703c. (FIG. 10)

Following the method described in Example 98, compound 503 (0.36 g, 0.53 mmol) was activated with TBTU (0.178 g, 0.55 mmol) and N,N-diisopropylethylamine (0.164 g, 1.27 mmol) in a mixture of pyridine (0.85 mL) and acetonitrile (6 mL) and reacted with aminopropyl CPG (15.0 g) in anhydrous acetonitrile (68 mL). The solid support was capped with N-methylimidazole (1.4 mL) and acetic anhydride (1.4 mL), filtered off, washed with acetonitrile (5×68 mL), and dried to give the loading of 32.0 μmol/g.

### Example 100

### (rel-(1R,3-endo,5S,6S,7R)-7-(bis(4-methoxyphenyl)(phenyl)methoxy)-3-isobutyryloxy-8-benzyl-8-azabicyclo[3.2.1]octane-6-yl) hemisuccinate, covalently bound to aminopropyl CPG1000, Solid support 705c. (FIG. 10)

Following the method described in Example 98, compound 505 (0.71 g, 0.99 mmol) was activated with TBTU (0.334 g, 1.04 mmol) and N,N-diisopropylethylamine (0.307 g, 2.38 mmol) in a mixture of pyridine (1.60 mL) and acetonitrile (10 mL) and reacted with aminopropyl CPG (20.0 g) in anhydrous acetonitrile (90 mL). The solid support was capped with N-methylimidazole (2.6 mL) and acetic anhydride (2.6 mL), filtered off, washed with acetonitrile (5×90 mL), and dried to give the loading of 43.7 μmol/g.

### Example 101

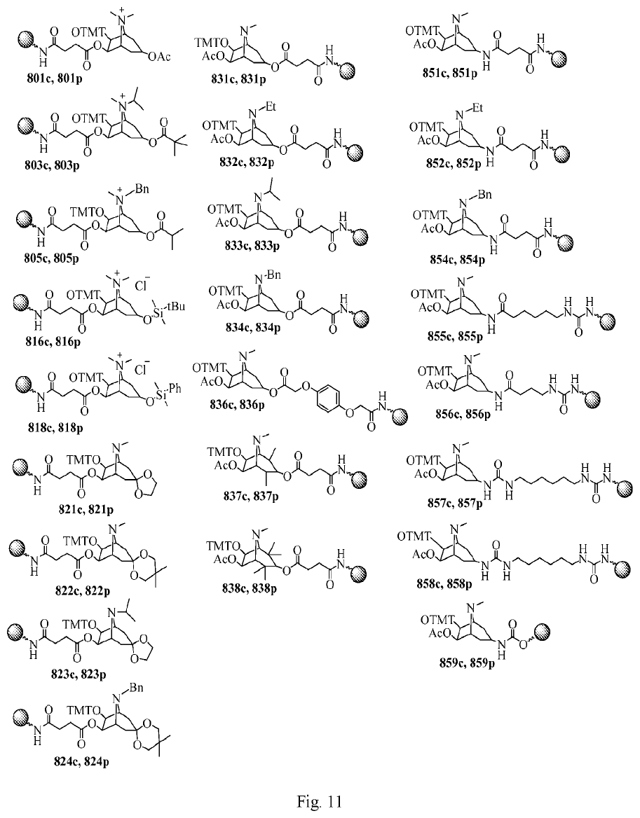

### (rel-(1R,3-endo,5S,6S,7R)-7-(tris(4-methoxyphenyl)methoxy)-3-acetoxy-8-methyl-8-azabicyclo[3.2.1]octane-6-yl) hemisuccinate, covalently bound to aminopropyl CPG1000, Solid support 801c. (FIG. 11)

Following the method described in Example 98, compound 601 (0.8 g, 1.24 mmol) was activated with TBTU (0.417 g, 1.30 mmol) and N,N-diisopropylethylamine (0.384 g, 2.97 mmol) in a mixture of pyridine (2.00 mL) and acetonitrile (13 mL) and reacted with aminopropyl CPG (25.0 g) in anhydrous acetonitrile (113 mL). The solid support was capped with N-methylimidazole (3.3 mL) and acetic anhydride (3.3 mL), filtered off, washed with acetonitrile (5×113 mL), and dried to give the loading of 44.2 μmol/g.

### Example 102

### (rel-(1R,3-endo,5S,6S,7R)-7-(tris(4-methoxyphenyl)methoxy)-3-pivaloyloxy-8-(i-propyl)-8-azabicyclo[3.2.1]octane-6-yl) hemisuccinate, covalently bound to aminopropyl CPG1000, Solid support 803c. (FIG. 11)

Following the method described in Example 98, compound 603 (0.53 g, 0.74 mmol) was activated with TBTU (0.250 g, 0.78 mmol) and N,N-diisopropylethylamine (0.23 g, 1.78 mmol) in a mixture of pyridine (1.20 mL) and acetonitrile (8 mL) and reacted with aminopropyl CPG (15.0 g) in anhydrous acetonitrile (68 mL). The solid support was capped with N-methylimidazole (2.0 mL) and acetic anhydride (2 mL), filtered off, washed with acetonitrile (5×68 mL), and dried to give the loading of 44.8 μmol/g.

### Example 103

### (rel-(1R,3-endo,5S,6S,7R)-7-(tris(4-methoxyphenyl)methoxy)-3-isobutyryloxy-8-methyl-8-azabicyclo[3.2.1]octane-6-yl) hemisuccinate, covalently bound to aminopropyl CPG500, Solid support 805c

Following the method described in Example 98, compound 605 (1.05 g, 1.4 mmol) was activated with TBTU (0.473 g, 1.47 mmol) and N,N-diisopropylethylamine (0.435 g, 3.37 mmol) in a mixture of pyridine (2.27 mL) and acetonitrile (15 mL) and reacted with aminopropyl CPG (17.0 g) in anhydrous acetonitrile (77 mL). The solid support was capped with N-methylimidazole (3.7 mL) and acetic anhydride (3.7 mL), filtered off, washed with acetonitrile (5×77 mL), and dried to give the loading of 73.1 μmol/g.

### Example 104

### (rel-(1R,3-endo,5S,6S,7R)-7-(tris(4-methoxyphenyl)methoxy)-3-[(t-butyl)dimethylsilyloxy]-8-methyl-8-azabicyclo[3.2.1]octane-6-yl) hemisuccinate, covalently bound to aminopropyl CPG1500, Solid support 816c. (FIG. 11)

Following the method described in Example 98, compound 616 (0.3 g, 0.42 mmol) was activated with TBTU (0.142 g, 0.44 mmol) and N,N-diisopropylethylamine (0.131 g, 1.01 mmol) in a mixture of pyridine (0.68 mL) and acetonitrile (4 mL) and reacted with aminopropyl CPG (12.0 g) in anhydrous acetonitrile (54 mL). The solid support was capped with N-methylimidazole (1.1 mL) and acetic anhydride (1.1 mL), filtered off, washed with acetonitrile (5×54 mL), and dried to give the loading of 32.4 μmol/g.

### Example 105

### (rel-(1R,3-endo,5S,6S,7R)-7-(tris(4-methoxyphenyl)methoxy)-3-[(t-butyl) diphenylsilyloxy]-8-methyl-8-azabicyclo[3.2.1]octane-6-yl) hemisuccinate, covalently bound to aminopropyl CPG1000, Solid support 818c. (FIG. 11)

Following the method described in Example 98, compound 618 (0.29 g, 0.35 mmol) was activated with TBTU (0.117 g, 0.36 mmol) and N,N-diisopropylethylamine (0.107 g, 0.83 mmol) in a mixture of pyridine (0.56 mL) and acetonitrile (4 mL) and reacted with aminopropyl CPG (7.0 g) in anhydrous acetonitrile (32 mL). The solid support was capped with N-methylimidazole (0.9 mL) and acetic anhydride (0.9 mL), filtered off, washed with acetonitrile (5× 32 mL), and dried to give the loading of 43.8 μmol/g.

### Example 106

### (rel-(1R,3-endo,5S,6S,7R)-7-(tris(4-methoxyphenyl)methoxy)-8-methyl-8-azaspiro[bicyclo[3.2.1]octane-3,2′-[1,3]dioxolane]-6-yl) hemisuccinate, covalently bound to aminopropyl CPG500, Solid support 821c. (FIG. 11)

Following the method described in Example 98, compound 621 (0.85 g, 1.32 mmol) was activated with TBTU (0.445 g, 1.39 mmol) and N,N-diisopropylethylamine (0.409 g, 3.17 mmol) in a mixture of pyridine (2.14 mL) and acetonitrile (14 mL) and reacted with aminopropyl CPG (16.0 g) in anhydrous acetonitrile (72 mL). The solid support was capped with N-methylimidazole (3.5 mL) and acetic anhydride (3.5 mL), filtered off, washed with acetonitrile (5×72 mL), and dried to give the loading of 74.4 μmol/g.

### Example 107

### (rel-(1R,3-endo,5S,6S,7R)-7-(tris(4-methoxyphenyl)methoxy)-8-methyl-8-azaspiro[bicyclo[3.2.1]octane-3,2′-[1,3]dioxane]-6-yl) hemisuccinate, covalently bound to aminopropyl CPG1000, Solid support 822c. (FIG. 11)

Following the method described in Example 98, compound 622 (0.68 g, 0.99 mmol) was activated with TBTU (0.334 g, 1.04 mmol) and N,N-diisopropylethylamine (0.307 g, 2.38 mmol) in a mixture of pyridine (1.60 mL) and acetonitrile (10 mL) and reacted with aminopropyl CPG (20.0 g) in anhydrous acetonitrile (90 mL). The solid support was capped with N-methylimidazole (2.6 mL) and acetic anhydride (2.6 mL), filtered off, washed with acetonitrile (5×90 mL), and dried to give the loading of 44.1 μmol/g.

### Example 108

### (rel-(1R,3-endo,5S,6S,7R)-7-(tris(4-methoxyphenyl)methoxy)-8-(i-propyl)-8-azaspiro[bicyclo[3.2.1]octane-3,2′-[1,3]dioxolane]-6-yl) hemisuccinate, covalently bound to aminopropyl CPG1500, Solid support 823c. (FIG. 11)

Following the method described in Example 98, compound 623 (0.55 g, 0.81 mmol) was activated with TBTU (0.273 g, 0.85 mmol) and N,N-diisopropylethylamine (0.251 g, 1.94 mmol) in a mixture of pyridine (1.31 mL) and acetonitrile (8 mL) and reacted with aminopropyl CPG (23.0 g) in anhydrous acetonitrile (104 mL). The solid support was capped with N-methylimidazole (2.1 mL) and acetic anhydride (2.1 mL), filtered off, washed with acetonitrile (5×104 mL), and dried to give the loading of 31.6 μmol/g.

### Example 109

### (rel-(1R,3-endo,5S,6S,7R)-7-(tris(4-methoxyphenyl)methoxy)-8-benzyl-8-azaspiro[bicyclo[3.2.1]octane-3,2′-[1,3]dioxane]-6-yl) hemisuccinate, covalently bound to aminopropyl CPG500, Solid support 824c. (FIG. 11)

Following the method described in Example 98, compound 624 (1.26 g, 1.65 mmol) was activated with TBTU (0.556 g, 1.73 mmol) and N,N-diisopropylethylamine (0.512 g, 3.96 mmol) in a mixture of pyridine (2.67 mL) and acetonitrile (17 mL) and reacted with aminopropyl CPG (20.0 g) in anhydrous acetonitrile (90 mL). The solid support was capped with N-methylimidazole (4.3 mL) and acetic anhydride (4.4 mL), filtered off, washed with acetonitrile (5×90 mL), and dried to give the loading of 72.9 μmol/g.

### Example 110

### (rel-(1R,3-endo,5S,6S,7R)-7-(bis(4-methoxyphenyl)(phenyl)methoxy)-6-acetoxy-8-methyl-8-azabicyclo[3.2.1]octane-3-yl) hemisuccinate, covalently bound to aminopropyl CPG500, Solid support 731c. (FIG. 10)

Following the method described in Example 98, compound 531 (2.55 g, 4.13 mmol) was activated with TBTU (1.391 g, 4.33 mmol) and N,N-diisopropylethylamine (1.28 g, 9.9 mmol) in a mixture of pyridine (6.67 mL) and acetonitrile (43 mL) and reacted with aminopropyl CPG (50.0 g) in anhydrous acetonitrile (225 mL). The solid support was capped with N-methylimidazole (10.9 mL) and acetic anhydride (10.9 mL), filtered off, washed with acetonitrile (5×225 mL), and dried to give the loading of 73.9 μmol/g.

### Example 111

### (rel-(1R,3-endo,5S,6S,7R)-7-(bis(4-methoxyphenyl)(phenyl)methoxy)-6-acetoxy-8-ethyl-8-azabicyclo[3.2.1]octane-3-yl) hemisuccinate, covalently bound to aminopropyl CPG1000, Solid support 732c. (FIG. 10)

Following the method described in Example 98, compound 532 (0.78 g, 1.24 mmol) was activated with TBTU (0.417 g, 1.30 mmol) and N,N-diisopropylethylamine (0.384 g, 2.97 mmol) in a mixture of pyridine (2.00 mL) and acetonitrile (13 mL) and reacted with aminopropyl CPG (25.0 g) in anhydrous acetonitrile (113 mL). The solid support was capped with N-methylimidazole (3.3 mL) and acetic anhydride (3.3 mL), filtered off, washed with acetonitrile (5×113 mL), and dried to give the loading of 44.4 μmol/g.

### Example 112

### (rel-(1R,3-endo,5S,6S,7R)-7-(bis(4-methoxyphenyl)(phenyl)methoxy)-6-acetoxy-8-(i-propyl)-8-azabicyclo[3.2.1]octane-3-yl) hemisuccinate, covalently bound to aminopropyl CPG1500, Solid support 733c. (FIG. 10)

Following the method described in Example 98, compound 533 (0.5 g, 0.77 mmol) was activated with TBTU (0.261 g, 0.81 mmol) and N,N-diisopropylethylamine (0.24 g, 1.86 mmol) in a mixture of pyridine (1.25 mL) and acetonitrile (8 mL) and reacted with aminopropyl CPG (22.0 g) in anhydrous acetonitrile (99 mL). The solid support was capped with N-methylimidazole (2.0 mL) and acetic anhydride (2 mL), filtered off, washed with acetonitrile (5×99 mL), and dried to give the loading of 31.8 μmol/g.

### Example 113

### (rel-(1R,3-endo,5S,6S,7R)-7-(bis(4-methoxyphenyl)(phenyl)methoxy)-6-acetoxy-8-benzyl-8-azabicyclo[3.2.1]octane-3-yl) hemisuccinate, covalently bound to aminopropyl CPG1000, Solid support 734c. (FIG. 10)

Following the method described in Example 98, compound 534 (0.69 g, 0.99 mmol) was activated with TBTU (0.334 g, 1.04 mmol) and N,N-diisopropylethylamine (0.307 g, 2.38 mmol) in a mixture of pyridine (1.60 mL) and acetonitrile (10 mL) and reacted with aminopropyl CPG (20.0 g) in anhydrous acetonitrile (90 mL). The solid support was capped with N-methylimidazole (2.6 mL) and acetic anhydride (2.6 mL), filtered off, washed with acetonitrile (5×90 mL), and dried to give the loading of 41.5 μmol/g.

### Example 114

### (rel-(1R,3-endo,5S,6S,7R)-7-(bis(4-methoxyphenyl)(phenyl)methoxy)-6-acetoxy-8-methyl-8-azabicyclo[3.2.1]octane-3-yl) diglycolate, covalently bound to aminopropyl CPG500, Solid support 735c. (FIG. 10)

Following the method described in Example 98, compound 535 (1.44 g, 2.27 mmol) was activated with TBTU (0.765 g, 2.38 mmol) and N,N-diisopropylethylamine (0.704 g, 5.45 mmol) in a mixture of pyridine (3.67 mL) and acetonitrile (24 mL) and reacted with aminopropyl CPG (27.5 g) in anhydrous acetonitrile (124 mL). The solid support was capped with N-methylimidazole (6.0 mL) and acetic anhydride (6 mL), filtered off, washed with acetonitrile (5×124 mL), and dried to give the loading of 72.6 μmol/g.

### Example 115

### (rel-(1R,3-endo,5S,6S,7R)-7-(bis(4-methoxyphenyl)(phenyl)methoxy)-6-acetoxy-2,4,8-trimethyl-8-azabicyclo[3.2.1]octane-3-yl) hemisuccinate, covalently bound to aminopropyl CPG500, Solid support 737c. (FIG. 10)

Following the method described in Example 98, compound 537 (1.07 g, 1.65 mmol) was activated with TBTU (0.556 g, 1.73 mmol) and N,N-diisopropylethylamine (0.512 g, 3.96 mmol) in a mixture of pyridine (2.67 mL) and acetonitrile (17 mL) and reacted with aminopropyl CPG (20.0 g) in anhydrous acetonitrile (90 mL). The solid support was capped with N-methylimidazole (4.3 mL) and acetic anhydride (4.4 mL), filtered off, washed with acetonitrile (5×90 mL), and dried to give the loading of 74.9 μmol/g.

### Example 116

### (rel-(1R,3-endo,5S,6S,7R)-7-(bis(4-methoxyphenyl)(phenyl)methoxy)-6-acetoxy-2,2,4,4,8-pentamethyl-8-azabicyclo[3.2.1]octane-3-yl) hemisuccinate, covalently bound to aminopropyl CPG500, Solid support 738c. (FIG. 10)

Following the method described in Example 98, compound 538 (1.39 g, 2.06 mmol) was activated with TBTU (0.695 g, 2.17 mmol) and N,N-diisopropylethylamine (0.64 g, 4.95 mmol) in a mixture of pyridine (3.34 mL) and acetonitrile (22 mL) and reacted with aminopropyl CPG (25.0 g) in anhydrous acetonitrile (113 mL). The solid support was capped with N-methylimidazole (5.4 mL) and acetic anhydride (5.4 mL), filtered off, washed with acetonitrile (5×113 mL), and dried to give the loading of 73.5 μmol/g.

### Example 117

### (rel-(1R,3-endo,5S,6S,7R)-7-(tris(4-methoxyphenyl)methoxy)-6-acetoxy-8-methyl-8-azabicyclo[3.2.1]octane-3-yl) hemisuccinate, covalently bound to aminopropyl CPG500, Solid support 831c. (FIG. 11)

Following the method described in Example 98, compound 631 (1.07 g, 1.65 mmol) was activated with TBTU (0.556 g, 1.73 mmol) and N,N-diisopropylethylamine (0.512 g, 3.96 mmol) in a mixture of pyridine (2.67 mL) and acetonitrile (17 mL) and reacted with aminopropyl CPG (20.0 g) in anhydrous acetonitrile (90 mL). The solid support was capped with N-methylimidazole (4.3 mL) and acetic anhydride (4.4 mL), filtered off, washed with acetonitrile (5×90 mL), and dried to give the loading of 72.7 μmol/g.

### Example 118

### (rel-(1R,3-endo,5S,6S,7R)-7-(tris(4-methoxyphenyl)methoxy)-6-acetoxy-8-ethyl-8-azabicyclo[3.2.1]octane-3-yl) hemisuccinate, covalently bound to aminopropyl CPG1000, Solid support 832c. (FIG. 11)

Following the method described in Example 98, compound 632 (0.66 g, 0.99 mmol) was activated with TBTU (0.334 g, 1.04 mmol) and N,N-diisopropylethylamine (0.307 g, 2.38 mmol) in a mixture of pyridine (1.60 mL) and acetonitrile (10 mL) and reacted with aminopropyl CPG (20.0 g) in anhydrous acetonitrile (90 mL). The solid support was capped with N-methylimidazole (2.6 mL) and acetic anhydride (2.6 mL), filtered off, washed with acetonitrile (5×90 mL), and dried to give the loading of 42.2 μmol/g.

### Example 119

### (rel-(1R,3-endo,5S,6S,7R)-7-(tris(4-methoxyphenyl)methoxy)-6-acetoxy-8-(i-propyl)-8-azabicyclo[3.2.1]octane-3-yl) hemisuccinate, covalently bound to aminopropyl CPG1500, Solid support 833c. (FIG. 11)

Following the method described in Example 98, compound 633 (0.48 g, 0.7 mmol) was activated with TBTU (0.237 g, 0.74 mmol) and N,N-diisopropylethylamine (0.218 g, 1.69 mmol) in a mixture of pyridine (1.14 mL) and acetonitrile (7 mL) and reacted with aminopropyl CPG (20.0 g) in anhydrous acetonitrile (90 mL). The solid support was capped with N-methylimidazole (1.9 mL) and acetic anhydride (1.9 mL), filtered off, washed with acetonitrile (5×90 mL), and dried to give the loading of 31.5 μmol/g.

### Example 120

### (rel-(1R,3-endo,5S,6S,7R)-7-(tris(4-methoxyphenyl)methoxy)-6-acetoxy-8-benzyl-8-azabicyclo[3.2.1]octane-3-yl) hemisuccinate, covalently bound to aminopropyl CPG1000, Solid support 834c. (FIG. 11)

Following the method described in Example 98, compound 634 (0.7 g, 0.97 mmol) was activated with TBTU (0.325 g, 1.01 mmol) and N,N-diisopropylethylamine (0.299 g, 2.32 mmol) in a mixture of pyridine (1.56 mL) and acetonitrile (10 mL) and reacted with aminopropyl CPG (19.5 g) in anhydrous acetonitrile (88 mL). The solid support was capped with N-methylimidazole (2.5 mL) and acetic anhydride (2.6 mL), filtered off, washed with acetonitrile (5×88 mL), and dried to give the loading of 45.0 μmol/g.

### Example 121

### (rel-(1R,3-endo,5S,6S,7R)-7-(tris(4-methoxyphenyl)methoxy)-6-acetoxy-8-methyl-8-azabicyclo[3.2.1]octane-3-yl) [2-[4-(carboxymethoxy) phenoxy]acetate], covalently bound to aminopropyl CPG500, Solid support 836c. (FIG. 11)

Following the method described in Example 98, compound 636 (0.53 g, 0.7 mmol) was activated with TBTU (0.237 g, 0.74 mmol) and N,N-diisopropylethylamine (0.218 g, 1.69 mmol) in a mixture of pyridine (1.14 mL) and acetonitrile (7 mL) and reacted with aminopropyl CPG (20.0 g) in anhydrous acetonitrile (90 mL). The solid support was capped with N-methylimidazole (1.9 mL) and acetic anhydride (1.9 mL), filtered off, washed with acetonitrile (5×90 mL), and dried to give the loading of 33.6 μmol/g.

### Example 122

### (rel-(1R,3-endo,5S,6S,7R)-7-(tris(4-methoxyphenyl)methoxy)-6-acetoxy-2,4,8-trimethyl-8-azabicyclo[3.2.1]octane-3-yl) hemisuccinate, covalently bound to aminopropyl CPG500, Solid support 837c. (FIG. 11)

Following the method described in Example 98, compound 637 (1.12 g, 1.65 mmol) was activated with TBTU (0.556 g, 1.73 mmol) and N,N-diisopropylethylamine (0.512 g, 3.96 mmol) in a mixture of pyridine (2.67 mL) and acetonitrile (17 mL) and reacted with aminopropyl CPG (20.0 g) in anhydrous acetonitrile (90 mL). The solid support was capped with N-methylimidazole (4.3 mL) and acetic anhydride (4.4 mL), filtered off, washed with acetonitrile (5×90 mL), and dried to give the loading of 75.0 μmol/g.

### Example 123

### (rel-(1R,3-endo,5S,6S,7R)-7-(tris(4-methoxyphenyl)methoxy)-6-acetoxy-2,2,4,4,8-pentamethyl-8-azabicyclo[3.2.1]octane-3-yl) hemisuccinate, covalently bound to aminopropyl CPG500, Solid support 838c. (FIG. 11)

Following the method described in Example 98, compound 638 (1.1 g, 1.56 mmol) was activated with TBTU (0.527 g, 1.64 mmol) and N,N-diisopropylethylamine (0.485 g, 3.75 mmol) in a mixture of pyridine (2.53 mL) and acetonitrile (16 mL) and reacted with aminopropyl CPG (19.0 g) in anhydrous acetonitrile (85 mL). The solid support was capped with N-methylimidazole (4.1 mL) and acetic anhydride (4.1 mL), filtered off, washed with acetonitrile (5×85 mL), and dried to give the loading of 72.3 μmol/g.

### Example 124

### (rel-(1R,3-endo,5S,6S,7R)-7-(tris(4-methoxyphenyl)methoxy)-3-methoxy-8,8-dimethyl-8-azoniabicyclo[3.2.1]octane-6-yl) hemisuccinate, covalently bound to aminopropyl CPG1000, Solid support 871c. (FIG. 13)

Following the method described in Example 98, compound 671 (0.87 g, 1.09 mmol) was activated with TBTU (0.367 g, 1.14 mmol) and N,N-diisopropylethylamine (0.338 g, 2.61 mmol) in a mixture of pyridine (1.76 mL) and acetonitrile (11 mL) and reacted with aminopropyl CPG (22.0 g) in anhydrous acetonitrile (99 mL). The solid support was capped with N-methylimidazole (2.9 mL) and acetic anhydride (2.9 mL), filtered off, washed with acetonitrile (5×99 mL), and dried to give the loading of 42.7 μmol/g.

### Example 125

### (rel-(1R,3-endo,5S,6S,7R)-7-(tris(4-methoxyphenyl)methoxy)-3-benzyloxy-8-methyl-8-benzyl-8-azoniabicyclo[3.2.1]octane-6-yl) hemisuccinate, covalently bound to aminopropyl CPG500, Solid support 874c. (FIG. 13)

Following the method described in Example 98, compound 674 (1.72 g, 1.82 mmol) was activated with TBTU (0.612 g, 1.91 mmol) and N,N-diisopropylethylamine (0.563 g, 4.36 mmol) in a mixture of pyridine (2.94 mL) and acetonitrile (19 mL) and reacted with aminopropyl CPG (22.0 g) in anhydrous acetonitrile (99 mL). The solid support was capped with N-methylimidazole (4.8 mL) and acetic anhydride (4.8 mL), filtered off, washed with acetonitrile (5×99 mL), and dried to give the loading of 74.1 μmol/g.

### Example 126

### (rel-(1R,3-endo,5S,6S,7R)-7-(tris(4-methoxyphenyl)methoxy)-3-methoxy-8-benzyl-8-azabicyclo[3.2.1]octane-6-yl) hemisuccinate, covalently bound to aminopropyl CPG1000, Solid support 875c. (FIG. 13)

Following the method described in Example 98, compound 675 (0.99 g, 1.14 mmol) was activated with TBTU (0.384 g, 1.20 mmol) and N,N-diisopropylethylamine (0.353 g, 2.73 mmol) in a mixture of pyridine (1.84 mL) and acetonitrile (12 mL) and reacted with aminopropyl CPG (23.0 g) in anhydrous acetonitrile (104 mL). The solid support was capped with N-methylimidazole (3.0 mL) and acetic anhydride (3 mL), filtered off, washed with acetonitrile (5×104 mL), and dried to give the loading of 43.3 μmol/g.

### Example 127

### N-[(rel-(1R,3-endo,5S,6S,7R)-7-(bis(4-methoxyphenyl)(phenyl)methoxy)-6-acetoxy-8-methyl-8-azabicyclo[3.2.1]octane-3-yl)]succinamic acid, covalently bound to aminopropyl CPG500, Solid support 751c. (FIG. 10)

Following the method described in Example 98, compound 551 (0.81 g, 1.31 mmol) was activated with TBTU (0.442 g, 1.38 mmol) and N,N-diisopropylethylamine (0.407 g, 3.15 mmol) in a mixture of pyridine (2.12 mL) and acetonitrile (14 mL) and reacted with aminopropyl CPG (15.9 g) in anhydrous acetonitrile (72 mL). The solid support was capped with N-methylimidazole (3.5 mL) and acetic anhydride (3.5 mL), filtered off, washed with acetonitrile (5×72 mL), and dried to give the loading of 74.8 μmol/g.

### Example 128

### N-[(rel-(1R,3-endo,5S,6S,7R)-7-(bis(4-methoxyphenyl)(phenyl)methoxy)-6-acetoxy-8-(i-propyl)-8-azabicyclo[3.2.1]octane-3-yl)]succinamic acid, covalently bound to aminopropyl CPG1500, Solid support 753c. (FIG. 10)

Following the method described in Example 98, compound 553 (0.35 g, 0.54 mmol) was activated with TBTU (0.181 g, 0.56 mmol) and N,N-diisopropylethylamine (0.166 g, 1.29 mmol) in a mixture of pyridine (0.87 mL) and acetonitrile (6 mL) and reacted with aminopropyl CPG (15.2 g) in anhydrous acetonitrile (68 mL). The solid support was capped with N-methylimidazole (1.4 mL) and acetic anhydride (1.4 mL), filtered off, washed with acetonitrile (5×68 mL), and dried to give the loading of 32.0 μmol/g.

### Example 129

### N-[(rel-(1R,3-endo,5S,6S,7R)-7-(tris(4-methoxyphenyl)methoxy)-6-acetoxy-8-methyl-8-azabicyclo[3.2.1]octane-3-yl)]succinamic acid, covalently bound to aminopropyl CPG1500, Solid support 851c. (FIG. 11)

Following the method described in Example 98, compound 651 (0.28 g, 0.44 mmol) was activated with TBTU (0.148 g, 0.46 mmol) and N,N-diisopropylethylamine (0.137 g, 1.06 mmol) in a mixture of pyridine (0.71 mL) and acetonitrile (5 mL) and reacted with aminopropyl CPG (12.5 g) in anhydrous acetonitrile (56 mL). The solid support was capped with N-methylimidazole (1.2 mL) and acetic anhydride (1.2 mL), filtered off, washed with acetonitrile (5×56 mL), and dried to give the loading of 32.9 μmol/g.

### Example 130

### N-[(rel-(1R,3-endo,5S,6S,7R)-7-(tris(4-methoxyphenyl)methoxy)-6-acetoxy-8-methyl-8-azabicyclo[3.2.1]octane-3-yl)]succinamic acid, covalently bound to hydroxypropyl CPG500, Solid support 859c. (FIG. 11)

Following the method described in Example 98, compound 651 (0.67 g, 1.03 mmol) was activated with TBTU (0.348 g, 1.08 mmol) and N,N-diisopropylethylamine (0.32 g, 2.48 mmol) in a mixture of pyridine (1.67 mL) and acetonitrile (11 mL) and reacted with aminopropyl CPG (12.5 g) in anhydrous acetonitrile (56 mL). The solid support was capped with N-methylimidazole (2.7 mL) and acetic anhydride (2.7 mL), filtered off, washed with acetonitrile (5×56 mL), and dried to give the loading of 74.6 μmol/g.

### Example 131

### N-methyl-N-[(rel-(1R,3-endo,5S,6S,7R)-7-(tris(4-methoxyphenyl)methoxy)-6-acetoxy-8-methyl-8-azabicyclo[3.2.1]octane-3-yl)]succinamic acid, covalently bound to aminopropyl CPG500, Solid support 852c. (FIG. 11)

Following the method described in Example 98, compound 652 (0.66 g, 1 mmol) was activated with TBTU (0.338 g, 1.05 mmol) and N,N-diisopropylethylamine (0.311 g, 2.41 mmol) in a mixture of pyridine (1.62 mL) and acetonitrile (10 mL) and reacted with aminopropyl CPG (12.2 g) in anhydrous acetonitrile (55 mL). The solid support was capped with N-methylimidazole (2.6 mL) and acetic anhydride (2.7 mL), filtered off, washed with acetonitrile (5×55 mL), and dried to give the loading of 73.6 μmol/g.

### Example 132

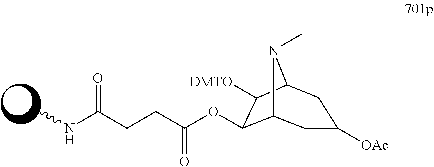

### (rel-(1R,3-endo,5S,6S,7R)-7-[(Bis(4-methoxyphenyl)(phenyl)methoxy]-3-acetoxy-8-methyl-aza[bicyclo[3.2.1]octane-6-yl) hemisuccinate, covalently bound to aminomethyl MPPS, 701p

TBTU (0.948 g, 2.95 mmol) was added to a solution of compound 401 (1.736 g, 2.81 mmol) and N,N-diisopropylethylamine (0.87 g, 2.95 mmol) in a mixture of anhydrous pyridine (4.55 mL) and acetonitrile (29 mL). The mixture was stirred for 15 min and transferred to a suspension of aminomethyl MPPS (7.3 g) in anhydrous acetonitrile (37 mL), and the resulting suspension was shaken for 4 h. The suspension was then charged with N-methylimidazole (7.4 mL) and acetic anhydride (7.4 mL) and was shaken again for 45 min. The solid support was filtered off, washed on the filter with acetonitrile (5×37 mL) and dried in vacuo. The loading of the finished solid support 701p (341 μmol/g) was determined, as applicable, by the standard di- or trimethoxytrityl assay as disclosed in Guzaev, A. P. and Pon, R. T. Curr. Protoc. Nucleic Acid Chem. 2013, 52, pp. 3.2.1-3.2.23.

### Example 133

### (rel-(1R,3-endo,5S,6S,7R)-7-(bis(4-methoxyphenyl)(phenyl)methoxy)-3-isobutyryloxy-8-benzyl-8-azabicyclo[3.2.1]octane-6-yl) hemisuccinate, covalently bound to aminomethyl MPPS, Solid support 705p. (FIG. 10)

Following the method described in Example 132, compound 505 (2.255 g, 3.12 mmol) was activated with TBTU (1.053 g, 3.28 mmol) and N,N-diisopropylethylamine (0.969 g, 7.50 mmol) in a mixture of pyridine (5.05 mL) and acetonitrile (33 mL) and reacted with aminomethyl MPPS (8.1 g) in anhydrous acetonitrile (41 mL). The solid support was capped with N-methylimidazole (8.2 mL) and acetic anhydride (8.3 mL), filtered off, washed with acetonitrile (5×41 mL), and dried to give the loading of 336 μmol/g.

### Example 134

### (rel-(1R,3-endo,5S,6S,7R)-7-(tris(4-methoxyphenyl)methoxy)-3-acetoxy-8-methyl-8-azabicyclo[3.2.1]octane-6-yl) hemisuccinate, covalently bound to aminomethyl MPPS, Solid support 801p. (FIG. 11)

Following the method described in Example 132, compound 601 (1.976 g, 3.05 mmol) was activated with TBTU (1.029 g, 3.20 mmol) and N,N-diisopropylethylamine (0.946 g, 7.32 mmol) in a mixture of pyridine (4.94 mL) and acetonitrile (32 mL) and reacted with aminomethyl MPPS (7.9 g) in anhydrous acetonitrile (40 mL). The solid support was capped with N-methylimidazole (8.0 mL) and acetic anhydride (8.1 mL), filtered off, washed with acetonitrile (5×40 mL), and dried to give the loading of 336 μmol/g.

### Example 135

### (rel-(1R,3-endo,5S,6S,7R)-7-(tris(4-methoxyphenyl)methoxy)-3-pivaloyloxy-8-(i-propyl)-8-azabicyclo[3.2.1]octane-6-yl) hemisuccinate, covalently bound to aminomethyl MPPS, Solid support 803p. (FIG. 11)

Following the method described in Example 132, compound 603 (2.531 g, 3.53 mmol) was activated with TBTU (1.189 g, 3.70 mmol) and N,N-diisopropylethylamine (1.094 g, 8.46 mmol) in a mixture of pyridine (5.70 mL) and acetonitrile (37 mL) and reacted with aminomethyl MPPS (9.2 g) in anhydrous acetonitrile (46 mL). The solid support was capped with N-methylimidazole (9.3 mL) and acetic anhydride (9.3 mL), filtered off, washed with acetonitrile (5× 46 mL), and dried to give the loading of 320 μmol/g.

### Example 136

### (rel-(1R,3-endo,5S,6S,7R)-7-(tris(4-methoxyphenyl)methoxy)-3-isobutyryloxy-8-methyl-8-azabicyclo[3.2.1]octane-6-yl) hemisuccinate, covalently bound to aminomethyl MPPS, Solid support 805p

Following the method described in Example 132, compound 605 (1.892 g, 2.52 mmol) was activated with TBTU (0.848 g, 2.64 mmol) and N,N-diisopropylethylamine (0.781 g, 6.04 mmol) in a mixture of pyridine (4.07 mL) and acetonitrile (26 mL) and reacted with aminomethyl MPPS (6.5 g) in anhydrous acetonitrile (33 mL). The solid support was capped with N-methylimidazole (6.6 mL) and acetic anhydride (6.6 mL), filtered off, washed with acetonitrile (5×33 mL), and dried to give the loading of 333 μmol/g.

### Example 137

### (rel-(1R,3-endo,5S,6S,7R)-7-(tris(4-methoxyphenyl)methoxy)-3-[(t-butyl)dimethylsilyloxy]-8-methyl-8-azabicyclo[3.2.1]octane-6-yl) hemisuccinate, covalently bound to aminomethyl MPPS, Solid support 816p. (FIG. 11)

Following the method described in Example 132, compound 616 (2.277 g, 3.16 mmol) was activated with TBTU (1.066 g, 3.32 mmol) and N,N-diisopropylethylamine (0.981 g, 7.59 mmol) in a mixture of pyridine (5.12 mL) and acetonitrile (33 mL) and reacted with aminomethyl MPPS (8.2 g) in anhydrous acetonitrile (41 mL). The solid support was capped with N-methylimidazole (8.3 mL) and acetic anhydride (8.4 mL), filtered off, washed with acetonitrile (5× 41 mL), and dried to give the loading of 326 μmol/g.

### Example 138

### (rel-(1R,3-endo,5S,6S,7R)-7-(tris(4-methoxyphenyl)methoxy)-3-[(t-butyl) diphenylsilyloxy]-8-methyl-8-azabicyclo[3.2.1]octane-6-yl) hemisuccinate, covalently bound to aminomethyl MPPS, Solid support 818p. (FIG. 11)

Following the method described in Example 132, compound 618 (2.670 g, 3.16 mmol) was activated with TBTU (1.066 g, 3.32 mmol) and N,N-diisopropylethylamine (0.981 g, 7.59 mmol) in a mixture of pyridine (5.12 mL) and acetonitrile (33 mL) and reacted with aminomethyl MPPS (8.2 g) in anhydrous acetonitrile (41 mL). The solid support was capped with N-methylimidazole (8.3 mL) and acetic anhydride (8.4 mL), filtered off, washed with acetonitrile (5× 41 mL), and dried to give the loading of 334 μmol/g.

### Example 139

### (rel-(1R,3-endo,5S,6S,7R)-7-(tris(4-methoxyphenyl)methoxy)-8-methyl-8-azaspiro[bicyclo[3.2.1]octane-3,2′-[1,3]dioxolane]-6-yl) hemisuccinate, covalently bound to aminomethyl MPPS, Solid support 821p. (FIG. 11)

Following the method described in Example 132, compound 621 (1.308 g, 2.02 mmol) was activated with TBTU (0.681 g, 2.12 mmol) and N,N-diisopropylethylamine (0.626 g, 4.85 mmol) in a mixture of pyridine (3.27 mL) and acetonitrile (21 mL) and reacted with aminomethyl MPPS (5.2 g) in anhydrous acetonitrile (26 mL). The solid support was capped with N-methylimidazole (5.3 mL) and acetic anhydride (5.3 mL), filtered off, washed with acetonitrile (5×26 mL), and dried to give the loading of 332 μmol/g.

### Example 140

### (rel-(1R,3-endo,5S,6S,7R)-7-(tris(4-methoxyphenyl)methoxy)-8-methyl-8-azaspiro[bicyclo[3.2.1]octane-3,2′-[1,3]dioxane]-6-yl) hemisuccinate, covalently bound to aminomethyl MPPS, Solid support 822p. (FIG. 11)

Following the method described in Example 132, compound 622 (2.363 g, 3.43 mmol) was activated with TBTU (1.155 g, 3.60 mmol) and N,N-diisopropylethylamine (1.063 g, 8.22 mmol) in a mixture of pyridine (5.54 mL) and acetonitrile (36 mL) and reacted with aminomethyl MPPS (8.9 g) in anhydrous acetonitrile (45 mL). The solid support was capped with N-methylimidazole (9.0 mL) and acetic anhydride (9.1 mL), filtered off, washed with acetonitrile (5×45 mL), and dried to give the loading of 324 μmol/g.

### Example 141

### (rel-(1R,3-endo,5S,6S,7R)-7-(tris(4-methoxyphenyl)methoxy)-8-(i-propyl)-8-azaspiro[bicyclo[3.2.1]octane-3,2′-[1,3]dioxolane]-6-yl) hemisuccinate, covalently bound to aminomethyl MPPS, Solid support 823p. (FIG. 11)

Following the method described in Example 132, compound 623 (1.302 g, 1.93 mmol) was activated with TBTU (0.649 g, 2.02 mmol) and N,N-diisopropylethylamine (0.598 g, 4.62 mmol) in a mixture of pyridine (3.12 mL) and acetonitrile (20 mL) and reacted with aminomethyl MPPS (5.0 g) in anhydrous acetonitrile (25 mL). The solid support was capped with N-methylimidazole (5.1 mL) and acetic anhydride (5.1 mL), filtered off, washed with acetonitrile (5×25 mL), and dried to give the loading of 324 μmol/g.

### Example 142

### (rel-(1R,3-endo,5S,6S,7R)-7-(tris(4-methoxyphenyl)methoxy)-8-benzyl-8-azaspiro[bicyclo[3.2.1]octane-3,2′-[1,3]dioxane]-6-yl) hemisuccinate, covalently bound to aminomethyl MPPS, Solid support 824p. (FIG. 11)

Following the method described in Example 132, compound 624 (2.721 g, 3.55 mmol) was activated with TBTU (1.198 g, 3.73 mmol) and N,N-diisopropylethylamine (1.102 g, 8.53 mmol) in a mixture of pyridine (5.75 mL) and acetonitrile (37 mL) and reacted with aminomethyl MPPS (9.2 g) in anhydrous acetonitrile (46 mL). The solid support was capped with N-methylimidazole (9.3 mL) and acetic anhydride (9.4 mL), filtered off, washed with acetonitrile (5×46 mL), and dried to give the loading of 324 μmol/g.

### Example 143

### (rel-(1R,3-endo,5S,6S,7R)-7-(bis(4-methoxyphenyl)(phenyl)methoxy)-6-acetoxy-8-methyl-8-azabicyclo[3.2.1]octane-3-yl) hemisuccinate, covalently bound to aminomethyl MPPS, Solid support 731p. (FIG. 10)

Following the method described in Example 132, compound 531 (1.633 g, 2.64 mmol) was activated with TBTU (0.891 g, 2.78 mmol) and N,N-diisopropylethylamine (0.820 g, 6.34 mmol) in a mixture of pyridine (4.28 mL) and acetonitrile (28 mL) and reacted with aminomethyl MPPS (6.9 g) in anhydrous acetonitrile (35 mL). The solid support was capped with N-methylimidazole (7.0 mL) and acetic anhydride (7.0 mL), filtered off, washed with acetonitrile (5×35 mL), and dried to give the loading of 343 μmol/g.

### Example 144

### (rel-(1R,3-endo,5S,6S,7R)-7-(bis(4-methoxyphenyl)(phenyl)methoxy)-6-acetoxy-8-ethyl-8-azabicyclo[3.2.1]octane-3-yl) hemisuccinate, covalently bound to aminomethyl MPPS, Solid support 732p. (FIG. 10)

Following the method described in Example 132, compound 532 (1.583 g, 2.51 mmol) was activated with TBTU (0.845 g, 2.63 mmol) and N,N-diisopropylethylamine (0.777 g, 6.01 mmol) in a mixture of pyridine (4.05 mL) and acetonitrile (26 mL) and reacted with aminomethyl MPPS (6.5 g) in anhydrous acetonitrile (33 mL). The solid support was capped with N-methylimidazole (6.6 mL) and acetic anhydride (6.6 mL), filtered off, washed with acetonitrile (5×33 mL), and dried to give the loading of 327 μmol/g.

### Example 145

### (rel-(1R,3-endo,5S,6S,7R)-7-(bis(4-methoxyphenyl)(phenyl)methoxy)-6-acetoxy-8-(i-propyl)-8-azabicyclo[3.2.1]octane-3-yl) hemisuccinate, covalently bound to aminomethyl MPPS, Solid support 733p. (FIG. 10)

Following the method described in Example 132, compound 533 (2.170 g, 3.36 mmol) was activated with TBTU (1.133 g, 3.53 mmol) and N,N-diisopropylethylamine (1.042 g, 8.06 mmol) in a mixture of pyridine (5.43 mL) and acetonitrile (35 mL) and reacted with aminomethyl MPPS (8.7 g) in anhydrous acetonitrile (44 mL). The solid support was capped with N-methylimidazole (8.8 mL) and acetic anhydride (8.9 mL), filtered off, washed with acetonitrile (5×44 mL), and dried to give the loading of 326 μmol/g.

### Example 146

### (rel-(1R,3-endo,5S,6S,7R)-7-(bis(4-methoxyphenyl)(phenyl)methoxy)-6-acetoxy-8-benzyl-8-azabicyclo[3.2.1]octane-3-yl) hemisuccinate, covalently bound to aminomethyl MPPS, Solid support 734p. (FIG. 10)

Following the method described in Example 132, compound 534 (2.163 g, 3.12 mmol) was activated with TBTU (1.051 g, 3.27 mmol) and N,N-diisopropylethylamine (0.967 g, 7.48 mmol) in a mixture of pyridine (5.04 mL) and acetonitrile (33 mL) and reacted with aminomethyl MPPS (8.1 g) in anhydrous acetonitrile (41 mL). The solid support was capped with N-methylimidazole (8.2 mL) and acetic anhydride (8.2 mL), filtered off, washed with acetonitrile (5×41 mL), and dried to give the loading of 331 μmol/g.

### Example 147

### (rel-(1R,3-endo,5S,6S,7R)-7-(bis(4-methoxyphenyl)(phenyl)methoxy)-6-acetoxy-8-methyl-8-azabicyclo[3.2.1]octane-3-yl) diglycolate, covalently bound to aminomethyl MPPS, Solid support 735p. (FIG. 10)

Following the method described in Example 132, compound 535 (1.762 g, 2.78 mmol) was activated with TBTU (0.937 g, 2.92 mmol) and N,N-diisopropylethylamine (0.862 g, 6.67 mmol) in a mixture of pyridine (4.50 mL) and acetonitrile (29 mL) and reacted with aminomethyl MPPS (7.2 g) in anhydrous acetonitrile (36 mL). The solid support was capped with N-methylimidazole (7.3 mL) and acetic anhydride (7.3 mL), filtered off, washed with acetonitrile (5×36 mL), and dried to give the loading of 335 μmol/g.

### Example 148

### (rel-(1R,3-endo,5S,6S,7R)-7-(bis(4-methoxyphenyl)(phenyl)methoxy)-6-acetoxy-2,4,8-trimethyl-8-azabicyclo[3.2.1]octane-3-yl) hemisuccinate, covalently bound to aminomethyl MPPS, Solid support 737p. (FIG. 10)

Following the method described in Example 132, compound 537 (2.086 g, 3.23 mmol) was activated with TBTU (1.089 g, 3.39 mmol) and N,N-diisopropylethylamine (1.002 g, 7.75 mmol) in a mixture of pyridine (5.22 mL) and acetonitrile (34 mL) and reacted with aminomethyl MPPS (8.4 g) in anhydrous acetonitrile (42 mL). The solid support was capped with N-methylimidazole (8.5 mL) and acetic anhydride (8.5 mL), filtered off, washed with acetonitrile (5×42 mL), and dried to give the loading of 326 μmol/g.

### Example 149

### (rel-(1R,3-endo,5S,6S,7R)-7-(bis(4-methoxyphenyl)(phenyl)methoxy)-6-acetoxy-2,2,4,4,8-pentamethyl-8-azabicyclo[3.2.1]octane-3-yl) hemisuccinate, covalently bound to aminomethyl MPPS, Solid support 738p. (FIG. 10)

Following the method described in Example 132, compound 538 (2.522 g, 3.74 mmol) was activated with TBTU (1.262 g, 3.93 mmol) and N,N-diisopropylethylamine (1.161 g, 8.98 mmol) in a mixture of pyridine (6.05 mL) and acetonitrile (39 mL) and reacted with aminomethyl MPPS (9.7 g) in anhydrous acetonitrile (49 mL). The solid support was capped with N-methylimidazole (9.8 mL) and acetic anhydride (9.9 mL), filtered off, washed with acetonitrile (5×49 mL), and dried to give the loading of 331 μmol/g.

### Example 150

### (rel-(1R,3-endo,5S,6S,7R)-7-(tris(4-methoxyphenyl)methoxy)-6-acetoxy-8-methyl-8-azabicyclo[3.2.1]octane-3-yl) hemisuccinate, covalently bound to aminomethyl MPPS, Solid support 831p. (FIG. 11)

Following the method described in Example 132, compound 631 (2.362 g, 3.65 mmol) was activated with TBTU (1.230 g, 3.83 mmol) and N,N-diisopropylethylamine (1.131 g, 8.75 mmol) in a mixture of pyridine (5.90 mL) and acetonitrile (38 mL) and reacted with aminomethyl MPPS (9.5 g) in anhydrous acetonitrile (48 mL). The solid support was capped with N-methylimidazole (9.6 mL) and acetic anhydride (9.6 mL), filtered off, washed with acetonitrile (5×48 mL), and dried to give the loading of 337 μmol/g.

### Example 151

### (rel-(1R,3-endo,5S,6S,7R)-7-(tris(4-methoxyphenyl)methoxy)-6-acetoxy-8-ethyl-8-azabicyclo[3.2.1]octane-3-yl) hemisuccinate, covalently bound to aminomethyl MPPS, Solid support 832p. (FIG. 11)

Following the method described in Example 132, compound 632 (1.897 g, 2.87 mmol) was activated with TBTU (0.966 g, 3.01 mmol) and N,N-diisopropylethylamine (0.889 g, 6.88 mmol) in a mixture of pyridine (4.64 mL) and acetonitrile (30 mL) and reacted with aminomethyl MPPS (7.4 g) in anhydrous acetonitrile (37 mL). The solid support was capped with N-methylimidazole (7.5 mL) and acetic anhydride (7.6 mL), filtered off, washed with acetonitrile (5×37 mL), and dried to give the loading of 341 μmol/g.

### Example 152

### (rel-(1R,3-endo,5S,6S,7R)-7-(tris(4-methoxyphenyl)methoxy)-6-acetoxy-8-(i-propyl)-8-azabicyclo[3.2.1]octane-3-yl) hemisuccinate, covalently bound to aminomethyl MPPS, Solid support 833p. (FIG. 11)

Following the method described in Example 132, compound 633 (2.259 g, 3.34 mmol) was activated with TBTU (1.127 g, 3.51 mmol) and N,N-diisopropylethylamine (1.037 g, 8.02 mmol) in a mixture of pyridine (5.41 mL) and acetonitrile (35 mL) and reacted with aminomethyl MPPS (8.7 g) in anhydrous acetonitrile (44 mL). The solid support was capped with N-methylimidazole (8.8 mL) and acetic anhydride (8.8 mL), filtered off, washed with acetonitrile (5×44 mL), and dried to give the loading of 338 μmol/g.

### Example 153

### (rel-(1R,3-endo,5S,6S,7R)-7-(tris(4-methoxyphenyl)methoxy)-6-acetoxy-8-benzyl-8-azabicyclo[3.2.1]octane-3-yl) hemisuccinate, covalently bound to aminomethyl MPPS, Solid support 834p. (FIG. 11)

Following the method described in Example 132, compound 634 (1.712 g, 2.36 mmol) was activated with TBTU (0.797 g, 2.48 mmol) and N,N-diisopropylethylamine (0.734 g, 5.68 mmol) in a mixture of pyridine (3.83 mL) and acetonitrile (25 mL) and reacted with aminomethyl MPPS (6.1 g) in anhydrous acetonitrile (31 mL). The solid support was capped with N-methylimidazole (6.2 mL) and acetic anhydride (6.2 mL), filtered off, washed with acetonitrile (5×31 mL), and dried to give the loading of 343 μmol/g.

### Example 154

### (rel-(1R,3-endo,5S,6S,7R)-7-(tris(4-methoxyphenyl)methoxy)-6-acetoxy-8-methyl-8-azabicyclo[3.2.1]octane-3-yl) [2-[4-(carboxymethoxy) phenoxy]acetate], covalently bound to aminomethyl MPPS, Solid support 836p. (FIG. 11)

Following the method described in Example 132, compound 636 (2.722 g, 3.60 mmol) was activated with TBTU (1.214 g, 3.78 mmol) and N,N-diisopropylethylamine (1.117 g, 8.64 mmol) in a mixture of pyridine (5.83 mL) and acetonitrile (38 mL) and reacted with aminomethyl MPPS (9.4 g) in anhydrous acetonitrile (47 mL). The solid support was capped with N-methylimidazole (9.5 mL) and acetic anhydride (9.5 mL), filtered off, washed with acetonitrile (5×47 mL), and dried to give the loading of 343 μmol/g.

### Example 155

### (rel-(1R,3-endo,5S,6S,7R)-7-(tris(4-methoxyphenyl)methoxy)-6-acetoxy-2,4,8-trimethyl-8-azabicyclo[3.2.1]octane-3-yl) hemisuccinate, covalently bound to aminomethyl MPPS, Solid support 837p. (FIG. 11)

Following the method described in Example 132, compound 637 (1.811 g, 2.68 mmol) was activated with TBTU (0.904 g, 2.81 mmol) and N,N-diisopropylethylamine (0.831 g, 6.43 mmol) in a mixture of pyridine (4.34 mL) and acetonitrile (28 mL) and reacted with aminomethyl MPPS (7.0 g) in anhydrous acetonitrile (35 mL). The solid support was capped with N-methylimidazole (7.1 mL) and acetic anhydride (7.1 mL), filtered off, washed with acetonitrile (5×35 mL), and dried to give the loading of 322 μmol/g.

### Example 156

### (rel-(1R,3-endo,5S,6S,7R)-7-(tris(4-methoxyphenyl)methoxy)-6-acetoxy-2,2,4,4,8-pentamethyl-8-azabicyclo[3.2.1]octane-3-yl) hemisuccinate, covalently bound to aminomethyl MPPS, Solid support 838p. (FIG. 11)

Following the method described in Example 132, compound 638 (1.959 g, 2.78 mmol) was activated with TBTU (0.938 g, 2.92 mmol) and N,N-diisopropylethylamine (0.863 g, 6.68 mmol) in a mixture of pyridine (4.50 mL) and acetonitrile (29 mL) and reacted with aminomethyl MPPS (7.2 g) in anhydrous acetonitrile (36 mL). The solid support was capped with N-methylimidazole (7.3 mL) and acetic anhydride (7.4 mL), filtered off, washed with acetonitrile (5×36 mL), and dried to give the loading of 341 μmol/g.

### Example 157

### (rel-(1R,3-endo,5S,6S,7R)-7-(tris(4-methoxyphenyl)methoxy)-3-methoxy-8,8-dimethyl-8-azoniabicyclo[3.2.1]octane-6-yl) hemisuccinate, covalently bound to aminomethyl MPPS, Solid support 871p. (FIG. 13)

Following the method described in Example 132, compound 671 (2.296 g, 2.89 mmol) was activated with TBTU (0.973 g, 3.03 mmol) and N,N-diisopropylethylamine (0.896 g, 6.93 mmol) in a mixture of pyridine (4.67 mL) and acetonitrile (30 mL) and reacted with aminomethyl MPPS (7.5 g) in anhydrous acetonitrile (38 mL). The solid support was capped with N-methylimidazole (7.6 mL) and acetic anhydride (7.6 mL), filtered off, washed with acetonitrile (5×38 mL), and dried to give the loading of 322 μmol/g.

### Example 158

### (rel-(1R,3-endo,5S,6S,7R)-7-(tris(4-methoxyphenyl)methoxy)-3-benzyloxy-8-methyl-8-benzyl-8-azoniabicyclo[3.2.1]octane-6-yl) hemisuccinate, covalently bound to aminomethyl MPPS, Solid support 874p. (FIG. 13)

Following the method described in Example 132, compound 674 (2.042 g, 2.16 mmol) was activated with TBTU (0.727 g, 2.26 mmol) and N,N-diisopropylethylamine (0.669 g, 5.17 mmol) in a mixture of pyridine (3.49 mL) and acetonitrile (23 mL) and reacted with aminomethyl MPPS (5.6 g) in anhydrous acetonitrile (28 mL). The solid support was capped with N-methylimidazole (5.7 mL) and acetic anhydride (5.7 mL), filtered off, washed with acetonitrile (5×28 mL), and dried to give the loading of 329 μmol/g.

### Example 159

### (rel-(1R,3-endo,5S,6S,7R)-7-(tris(4-methoxyphenyl)methoxy)-3-methoxy-8-benzyl-8-azabicyclo[3.2.1]octane-6-yl) hemisuccinate, covalently bound to aminomethyl MPPS, Solid support 875p. (FIG. 13)

Following the method described in Example 132, compound 675 (2.683 g, 3.08 mmol) was activated with TBTU (1.038 g, 3.23 mmol) and N,N-diisopropylethylamine (0.955 g, 7.39 mmol) in a mixture of pyridine (4.98 mL) and acetonitrile (32 mL) and reacted with aminomethyl MPPS (8.0 g) in anhydrous acetonitrile (40 mL). The solid support was capped with N-methylimidazole (8.1 mL) and acetic anhydride (8.1 mL), filtered off, washed with acetonitrile (5×40 mL), and dried to give the loading of 337 μmol/g.

### Example 160

### N-[(rel-(1R,3-endo,5S,6S,7R)-7-(bis(4-methoxyphenyl)(phenyl)methoxy)-6-acetoxy-8-methyl-8-azabicyclo[3.2.1]octane-3-yl)]succinamic acid, covalently bound to aminomethyl MPPS, Solid support 751p. (FIG. 10)

Following the method described in Example 132, compound 551 (1.585 g, 2.57 mmol) was activated with TBTU (0.866 g, 2.70 mmol) and N,N-diisopropylethylamine (0.797 g, 6.17 mmol) in a mixture of pyridine (4.16 mL) and acetonitrile (27 mL) and reacted with aminomethyl MPPS (6.7 g) in anhydrous acetonitrile (34 mL). The solid support was capped with N-methylimidazole (6.8 mL) and acetic anhydride (6.8 mL), filtered off, washed with acetonitrile (5×34 mL), and dried to give the loading of 346 μmol/g.

### Example 161

### N-[(rel-(1R,3-endo,5S,6S,7R)-7-(bis(4-methoxyphenyl)(phenyl)methoxy)-6-acetoxy-8-(i-propyl)-8-azabicyclo[3.2.1]octane-3-yl)]succinamic acid, covalently bound to aminomethyl MPPS, Solid support 753p. (FIG. 10)

Following the method described in Example 132, compound 553 (1.626 g, 2.52 mmol) was activated with TBTU (0.850 g, 2.65 mmol) and N,N-diisopropylethylamine (0.782 g, 6.05 mmol) in a mixture of pyridine (4.08 mL) and acetonitrile (26 mL) and reacted with aminomethyl MPPS (6.6 g) in anhydrous acetonitrile (33 mL). The solid support was capped with N-methylimidazole (6.6 mL) and acetic anhydride (6.7 mL), filtered off, washed with acetonitrile (5×33 mL), and dried to give the loading of 347 μmol/g.

### Example 162

### N-[(rel-(1R,3-endo,5S,6S,7R)-7-(tris(4-methoxyphenyl)methoxy)-6-acetoxy-8-methyl-8-azabicyclo[3.2.1]octane-3-yl)]succinamic acid, covalently bound to aminomethyl MPPS, Solid support 851p. (FIG. 11)

Following the method described in Example 132, compound 651 (1.508 g, 2.33 mmol) was activated with TBTU (0.786 g, 2.45 mmol) and N,N-diisopropylethylamine (0.724 g, 5.60 mmol) in a mixture of pyridine (3.77 mL) and acetonitrile (24 mL) and reacted with aminomethyl MPPS (6.1 g) in anhydrous acetonitrile (30 mL). The solid support was capped with N-methylimidazole (6.1 mL) and acetic anhydride (6.2 mL), filtered off, washed with acetonitrile (5×30 mL), and dried to give the loading of 352 μmol/g.

### Example 163

### N-[(rel-(1R,3-endo,5S,6S,7R)-7-(tris(4-methoxyphenyl)methoxy)-6-acetoxy-8-methyl-8-azabicyclo[3.2.1]octane-3-yl)]succinamic acid, covalently bound to hydroxymethyl MPPS, Solid support 851p. (FIG. 11)

Following the method described in Example 132, compound 651 (1.508 g, 2.33 mmol) was activated with TBTU (0.786 g, 2.45 mmol) and N,N-diisopropylethylamine (0.724 g, 5.60 mmol) in a mixture of pyridine (3.77 mL) and acetonitrile (24 mL) and reacted with aminomethyl MPPS (6.1 g) in anhydrous acetonitrile (30 mL). The solid support was capped with N-methylimidazole (6.1 mL) and acetic anhydride (6.2 mL), filtered off, washed with acetonitrile (5×30 mL), and dried to give the loading of 352 μmol/g.

### Example 164

### N-methyl-N-[(rel-(1R,3-endo,5S,6S,7R)-7-(tris(4-methoxyphenyl)methoxy)-6-acetoxy-8-methyl-8-azabicyclo[3.2.1]octane-3-yl)]succinamic acid, covalently bound to aminomethyl MPPS, Solid support 852p. (FIG. 11)

Following the method described in Example 132, compound 652 (1.526 g, 2.31 mmol) was activated with TBTU (0.778 g, 2.42 mmol) and N,N-diisopropylethylamine (0.716 g, 5.54 mmol) in a mixture of pyridine (3.73 mL) and acetonitrile (24 mL) and reacted with aminomethyl MPPS (6.0 g) in anhydrous acetonitrile (30 mL). The solid support was capped with N-methylimidazole (6.1 mL) and acetic anhydride (6.1 mL), filtered off, washed with acetonitrile (5×30 mL), and dried to give the loading of 353 μmol/g.

### Example 165

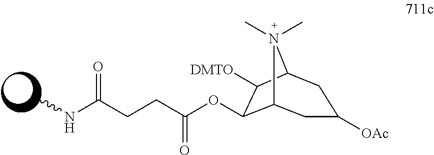

### (rel-(1R,3-endo,5S,6S,7R)-7-[(Bis(4-methoxyphenyl)(phenyl)methoxy]-3-acetoxy-8,8-dimethyl-azoniabicyclo[3.2.1]octane-6-yl) hemisuccinate, covalently bound to aminopropyl CPG500, 711c

Methyl iodide (0.4 M in MeCN, 43 mL) and N,N-diisopropylethylamine (0.535 g) were added to a solid support 701c (10 g). The resulting suspension was shaken for 12 h. The solid support was filtered off, washed on the filter with 0.25M DIPEA-HCl in acetonitrile (55 mL) and then with acetonitrile (5×50 mL) and dried in vacuo. The loading of the finished solid support 701c (69.0 μmol/g) was determined, as applicable, by the standard di- or trimethoxytrityl assay as disclosed in Guzaev, A. P. and Pon, R. T. Curr. Protoc. Nucleic Acid Chem. 2013, 52, pp. 3.2.1-3.2.23.

### Example 166

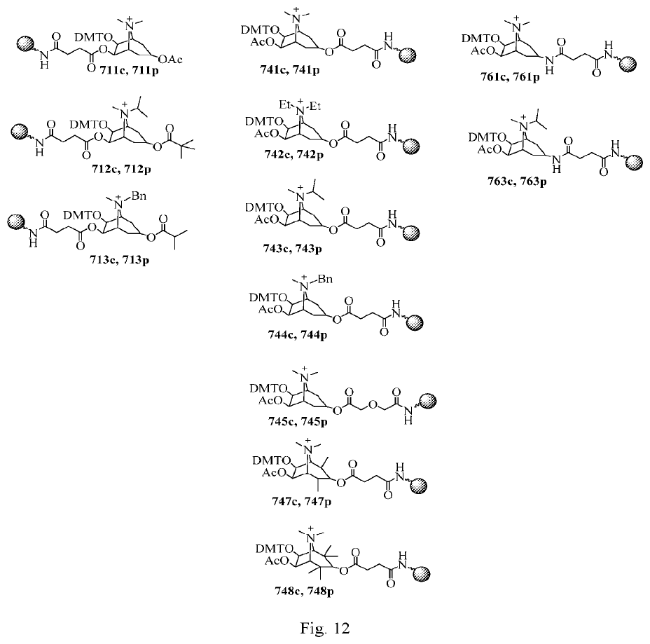

### (rel-(1R,3-endo,5S,6S,7R)-7-(bis(4-methoxyphenyl)(phenyl)methoxy)-3-pivaloyloxy-8-methyl-8-(i-propyl)-8-azoniabicyclo[3.2.1]octane-6-yl) hemisuccinate, covalently bound to aminopropyl CPG1500, Solid support 712c. (FIG. 12)

Following the method described in Example 165, solid support 703c (10.00 g) was alkylated with methyl iodide (0.35 M in acetonitrile, 20.013 mL) and N,N-diisopropylethylamine (0.248 g). The solid support was filtered off, washed with 025M DIPEA-HCl in acetonitrile (26 mL) and with acetonitrile (5×50 mL), and dried to give the loading of 32.0 μmol/g.

### Example 167

### (rel-(1R,3-endo,5S,6S,7R)-7-(bis(4-methoxyphenyl)(phenyl)methoxy)-3-isobutyryloxy-8-methyl-8-benzyl-8-azoniabicyclo[3.2.1]octane-6-yl) hemisuccinate, covalently bound to aminopropyl CPG1000, Solid support 713c. (FIG. 12)

Following the method described in Example 165, solid support 705c (10.00 g) was alkylated with methyl iodide (0.35 M in acetonitrile, 27.313 mL) and N,N-diisopropylethylamine (0.339 g). The solid support was filtered off, washed with 025M DIPEA-HCl in acetonitrile (35 mL) and with acetonitrile (5×50 mL), and dried to give the loading of 43.7 μmol/g.

### Example 168

### (rel-(1R,3-endo,5S,6S,7R)-7-(tris(4-methoxyphenyl)methoxy)-3-acetoxy-8,8-dimethyl-8-azabicyclo[3.2.1]octane-6-yl) hemisuccinate, covalently bound to aminopropyl CPG1000, Solid support 811c. (FIG. 13)

Following the method described in Example 165, solid support 801c (10.00 g) was alkylated with methyl iodide (0.35 M in acetonitrile, 27.625 mL) and N,N-diisopropylethylamine (0.343 g). The solid support was filtered off, washed with 025M DIPEA-HCl in acetonitrile (35 mL) and with acetonitrile (5×50 mL), and dried to give the loading of 44.2 μmol/g.

### Example 169

### (rel-(1R,3-endo,5S,6S,7R)-7-(tris(4-methoxyphenyl)methoxy)-3-pivaloyloxy-8-methyl-8-(i-propyl)-8-azoniabicyclo[3.2.1]octane-6-yl) hemisuccinate, covalently bound to aminopropyl CPG1000, Solid support 812c

Following the method described in Example 165, solid support 803c (10.00 g) was alkylated with methyl iodide (0.35 M in acetonitrile, 28.000 mL) and N,N-diisopropylethylamine (0.347 g). The solid support was filtered off, washed with 025M DIPEA-HCl in acetonitrile (36 mL) and with acetonitrile (5×50 mL), and dried to give the loading of 44.8 μmol/g.

### Example 170

### (rel-(1R,3-endo,5S,6S,7R)-7-(tris(4-methoxyphenyl)methoxy)-3-isobutyryloxy-8-methyl-8-benzyl-8-azoniabicyclo[3.2.1]octane-6-yl) hemisuccinate, covalently bound to aminopropyl CPG500, Solid support 813c. (FIG. 13)

Following the method described in Example 165, solid support 805c (10.00 g) was alkylated with methyl iodide (0.35 M in acetonitrile, 45.688 mL) and N,N-diisopropylethylamine (0.567 g). The solid support was filtered off, washed with 025M DIPEA-HCl in acetonitrile (58 mL) and with acetonitrile (5×50 mL), and dried to give the loading of 73.1 μmol/g.

### Example 171

### (rel-(1R,3-endo,5S,6S,7R)-7-(tris(4-methoxyphenyl)methoxy)-3-[(t-butyl)dimethylsilyloxy]-8,8-dimethyl-8-azoniabicyclo[3.2.1]octane-6-yl) hemisuccinate, covalently bound to aminopropyl CPG1500, Solid support 817c. (FIG. 13)

Following the method described in Example 165, solid support 816c (10.00 g) was alkylated with methyl iodide (0.35 M in acetonitrile, 20.250 mL) and N,N-diisopropylethylamine (0.251 g). The solid support was filtered off, washed with 025M DIPEA-HCl in acetonitrile (26 mL) and with acetonitrile (5×50 mL), and dried to give the loading of 32.4 μmol/g.

### Example 172

### (rel-(1R,3-endo,5S,6S,7R)-7-(tris(4-methoxyphenyl)methoxy)-3-[(t-butyl) diphenylsilyloxy]-8,8-dimethyl-8-azoniabicyclo[3.2.1]octane-6-yl) hemisuccinate, covalently bound to aminopropyl CPG1000, Solid support 819c. (FIG. 13)

Following the method described in Example 165, solid support 818c (10.00 g) was alkylated with methyl iodide (0.35 M in acetonitrile, 27.375 mL) and N,N-diisopropylethylamine (0.340 g). The solid support was filtered off, washed with 025M DIPEA-HCl in acetonitrile (35 mL) and with acetonitrile (5×50 mL), and dried to give the loading of 43.8 μmol/g.

### Example 173

### (rel-(1R,3-endo,5S,6S,7R)-7-(tris(4-methoxyphenyl)methoxy)-8,8-dimethyl-8-azoniaspiro[bicyclo[3.2.1]octane-3,2′-[1,3]dioxolane]-6-yl) hemisuccinate, covalently bound to aminopropyl CPG500, Solid support 825p. (FIG. 13)

Following the method described in Example 165, solid support 821c (10.00 g) was alkylated with methyl iodide (0.35 M in acetonitrile, 46.500 mL) and N,N-diisopropylethylamine (0.577 g). The solid support was filtered off, washed with 025M DIPEA-HCl in acetonitrile (60 mL) and with acetonitrile (5×50 mL), and dried to give the loading of 74.4 μmol/g.

### Example 174

### (rel-(1R,3-endo,5S,6S,7R)-7-(tris(4-methoxyphenyl)methoxy)-5′,5′,8-trimethyl-8-azoniaspiro[bicyclo[3.2.1]octane-3,2′-[1,3]dioxane]-6-yl) hemisuccinate, covalently bound to aminopropyl CPG1000, Solid support 826p. (FIG. 13)

Following the method described in Example 165, solid support 822c (10.00 g) was alkylated with methyl iodide (0.35 M in acetonitrile, 27.563 mL) and N,N-diisopropylethylamine (0.342 g). The solid support was filtered off, washed with 025M DIPEA-HCl in acetonitrile (35 mL) and with acetonitrile (5×50 mL), and dried to give the loading of 44.1 μmol/g.

### Example 175

### (rel-(1R,3-endo,5S,6S,7R)-7-(tris(4-methoxyphenyl)methoxy)-8-methyl-8-(i-propyl)-8-azoniaspiro[bicyclo[3.2.1]octane-3,2′-[1,3]dioxolane]-6-yl) hemisuccinate, covalently bound to aminopropyl CPG1500, Solid support 827p. (FIG. 13)

Following the method described in Example 165, solid support 823c (10.00 g) was alkylated with methyl iodide (0.35 M in acetonitrile, 19.750 mL) and N,N-diisopropylethylamine (0.245 g). The solid support was filtered off, washed with 025M DIPEA-HCl in acetonitrile (25 mL) and with acetonitrile (5×50 mL), and dried to give the loading of 31.6 μmol/g.

### Example 176

### (rel-(1R,3-endo,5S,6S,7R)-7-(tris(4-methoxyphenyl)methoxy)-5′,5′,8-trimethyl-8-azoniaspiro[bicyclo[3.2.1]octane-3,2′-[1,3]dioxane]-6-yl) hemisuccinate, covalently bound to aminopropyl CPG500, Solid support 828p. (FIG. 13)

Following the method described in Example 165, solid support 824c (10.00 g) was alkylated with methyl iodide (0.35 M in acetonitrile, 45.563 mL) and N,N-diisopropylethylamine (0.565 g). The solid support was filtered off, washed with 025M DIPEA-HCl in acetonitrile (58 mL) and with acetonitrile (5×50 mL), and dried to give the loading of 72.9 μmol/g.

### Example 177

### (rel-(1R,3-endo,5S,6S,7R)-7-(bis(4-methoxyphenyl)(phenyl)methoxy)-6-acetoxy-8,8-dimethyl-8-azoniabicyclo[3.2.1]octane-3-yl) hemisuccinate, covalently bound to aminopropyl CPG500, Solid support 741c. (FIG. 12)

Following the method described in Example 165, solid support 731c (10.00 g) was alkylated with methyl iodide (0.35 M in acetonitrile, 46.188 mL) and N,N-diisopropylethylamine (0.573 g). The solid support was filtered off, washed with 025M DIPEA-HCl in acetonitrile (59 mL) and with acetonitrile (5×50 mL), and dried to give the loading of 73.9 μmol/g.

### Example 178

### (rel-(1R,3-endo,5S,6S,7R)-7-(bis(4-methoxyphenyl)(phenyl)methoxy)-6-acetoxy-8,8-diethyl-8-azoniabicyclo[3.2.1]octane-3-yl) hemisuccinate, covalently bound to aminopropyl CPG1000, Solid support 742c. (FIG. 12)

Following the method described in Example 165, solid support 732c (10.00 g) was alkylated with ethyl iodide (0.35 M in acetonitrile, 27.750 mL) and N,N-diisopropylethylamine (0.344 g). The solid support was filtered off, washed with 025M DIPEA-HCl in acetonitrile (36 mL) and with acetonitrile (5×50 mL), and dried to give the loading of 44.4 μmol/g.

### Example 179

### (rel-(1R,3-endo,5S,6S,7R)-7-(bis(4-methoxyphenyl)(phenyl)methoxy)-6-acetoxy-8-methyl-8-(i-propyl)-8-azoniabicyclo[3.2.1]octane-3-yl) hemisuccinate, covalently bound to aminopropyl CPG1500, Solid support 743c. (FIG. 12)

Following the method described in Example 165, solid support 733c (10.00 g) was alkylated with methyl iodide (0.35 M in acetonitrile, 19.875 mL) and N,N-diisopropylethylamine (0.247 g). The solid support was filtered off, washed with 025M DIPEA-HCl in acetonitrile (25 mL) and with acetonitrile (5×50 mL), and dried to give the loading of 31.8 μmol/g.

### Example 180

### (rel-(1R,3-endo,5S,6S,7R)-7-(bis(4-methoxyphenyl)(phenyl)methoxy)-6-acetoxy-8-methyl-8-benzyl-8-azoniabicyclo[3.2.1]octane-3-yl) hemisuccinate, covalently bound to aminopropyl CPG1000, Solid support 744c. (FIG. 12)

Following the method described in Example 165, solid support 734c (10.00 g) was alkylated with methyl iodide (0.35 M in acetonitrile, 25.938 mL) and N,N-diisopropylethylamine (0.322 g). The solid support was filtered off, washed with 025M DIPEA-HCl in acetonitrile (33 mL) and with acetonitrile (5×50 mL), and dried to give the loading of 41.5 μmol/g.

### Example 181

### (rel-(1R,3-endo,5S,6S,7R)-7-(bis(4-methoxyphenyl)(phenyl)methoxy)-6-acetoxy-8,8-dimethyl-8-azoniabicyclo[3.2.1]octane-3-yl) diglycolate, covalently bound to aminopropyl CPG500, Solid support 745c. (FIG. 12)

Following the method described in Example 165, solid support 735c (10.00 g) was alkylated with methyl iodide (0.35 M in acetonitrile, 45.375 mL) and N,N-diisopropylethylamine (0.563 g). The solid support was filtered off, washed with 025M DIPEA-HCl in acetonitrile (58 mL) and with acetonitrile (5×50 mL), and dried to give the loading of 72.6 μmol/g.

### Example 182

### (rel-(1R,3-endo,5S,6S,7R)-7-(bis(4-methoxyphenyl)(phenyl)methoxy)-6-acetoxy-2,4,8,8-tetramethyl-8-azoniabicyclo[3.2.1]octane-3-yl) hemisuccinate, covalently bound to aminopropyl CPG500, Solid support 747c. (FIG. 12)

Following the method described in Example 165, solid support 737c (10.00 g) was alkylated with methyl iodide (0.35 M in acetonitrile, 46.813 mL) and N,N-diisopropylethylamine (0.581 g). The solid support was filtered off, washed with 025M DIPEA-HCl in acetonitrile (60 mL) and with acetonitrile (5×50 mL), and dried to give the loading of 74.9 μmol/g.

### Example 183

### (rel-(1R,3-endo,5S,6S,7R)-7-(bis(4-methoxyphenyl)(phenyl)methoxy)-6-acetoxy-2,2,4,4,8,8-hexamethyl-8-azoniabicyclo[3.2.1]octane-3-yl) hemisuccinate, covalently bound to aminopropyl CPG500, Solid support 748c. (FIG. 12)

Following the method described in Example 165, solid support 738c (10.00 g) was alkylated with methyl iodide (0.35 M in acetonitrile, 45.938 mL) and N,N-diisopropylethylamine (0.570 g). The solid support was filtered off, washed with 025M DIPEA-HCl in acetonitrile (59 mL) and with acetonitrile (5×50 mL), and dried to give the loading of 73.5 μmol/g.

### Example 184

### (rel-(1R,3-endo,5S,6S,7R)-7-(tris(4-methoxyphenyl)methoxy)-6-acetoxy-8,8-dimethyl-8-azoniabicyclo[3.2.1]octane-3-yl) hemisuccinate, covalently bound to aminopropyl CPG500, Solid support 841c. (FIG. 13)

Following the method described in Example 165, solid support 831c (10.00 g) was alkylated with methyl iodide (0.35 M in acetonitrile, 45.438 mL) and N,N-diisopropylethylamine (0.564 g). The solid support was filtered off, washed with 025M DIPEA-HCl in acetonitrile (58 mL) and with acetonitrile (5×50 mL), and dried to give the loading of 72.7 μmol/g.

### Example 185

### (rel-(1R,3-endo,5S,6S,7R)-7-(tris(4-methoxyphenyl)methoxy)-6-acetoxy-8,8-diethyl-8-azoniabicyclo[3.2.1]octane-3-yl) hemisuccinate, covalently bound to aminopropyl CPG1000, Solid support 842c. (FIG. 13)

Following the method described in Example 165, solid support 832c (10.00 g) was alkylated with ethyl iodide (0.35 M in acetonitrile, 26.375 mL) and N,N-diisopropylethylamine (0.327 g). The solid support was filtered off, washed with 025M DIPEA-HCl in acetonitrile (34 mL) and with acetonitrile (5×50 mL), and dried to give the loading of 42.2 μmol/g.

### Example 186

### (rel-(1R,3-endo,5S,6S,7R)-7-(tris(4-methoxyphenyl)methoxy)-6-acetoxy-8-methyl-8-(i-propyl)-8-azoniabicyclo[3.2.1]octane-3-yl) hemisuccinate, covalently bound to aminopropyl CPG1500, Solid support 843c. (FIG. 13)

Following the method described in Example 165, solid support 833c (10.00 g) was alkylated with methyl iodide (0.35 M in acetonitrile, 19.685 mL) and N,N-diisopropylethylamine (0.244 g). The solid support was filtered off, washed with 025M DIPEA-HCl in acetonitrile (25 mL) and with acetonitrile (5×50 mL), and dried to give the loading of 31.5 μmol/g.

### Example 187

### (rel-(1R,3-endo,5S,6S,7R)-7-(tris(4-methoxyphenyl)methoxy)-6-acetoxy-8-methyl-8-benzyl-8-azoniabicyclo[3.2.1]octane-3-yl) hemisuccinate, covalently bound to aminopropyl CPG1000, Solid support 844c. (FIG. 13)

Following the method described in Example 165, solid support 834c (10.00 g) was alkylated with methyl iodide (0.35 M in acetonitrile, 28.125 mL) and N,N-diisopropylethylamine (0.349 g). The solid support was filtered off, washed with 025M DIPEA-HCl in acetonitrile (36 mL) and with acetonitrile (5×50 mL), and dried to give the loading of 45.0 μmol/g.

### Example 188

### (rel-(1R,3-endo,5S,6S,7R)-7-(tris(4-methoxyphenyl)methoxy)-6-acetoxy-8,8-dimethyl-8-azoniabicyclo[3.2.1]octane-3-yl) [2-[4-(carboxymethoxy) phenoxy]acetate], covalently bound to aminopropyl CPG500, Solid support 846c. (FIG. 13)

Following the method described in Example 165, solid support 836c (10.00 g) was alkylated with methyl iodide (0.35 M in acetonitrile, 20.982 mL) and N,N-diisopropylethylamine (0.260 g). The solid support was filtered off, washed with 025M DIPEA-HCl in acetonitrile (27 mL) and with acetonitrile (5×50 mL), and dried to give the loading of 33.6 μmol/g.

### Example 189

### (rel-(1R,3-endo,5S,6S,7R)-7-(tris(4-methoxyphenyl)methoxy)-6-acetoxy-2,4,8,8-tetramethyl-8-azoniabicyclo[3.2.1]octane-3-yl) hemisuccinate, covalently bound to aminopropyl CPG500, Solid support 847c. (FIG. 13)

Following the method described in Example 165, solid support 837c (10.00 g) was alkylated with methyl iodide (0.35 M in acetonitrile, 46.875 mL) and N,N-diisopropylethylamine (0.582 g). The solid support was filtered off, washed with 025M DIPEA-HCl in acetonitrile (60 mL) and with acetonitrile (5×50 mL), and dried to give the loading of 75.0 μmol/g.

### Example 190

### (rel-(1R,3-endo,5S,6S,7R)-7-(tris(4-methoxyphenyl)methoxy)-6-acetoxy-2,2,4,4,8,8-hexamethyl-8-azoniabicyclo[3.2.1]octane-3-yl) hemisuccinate, covalently bound to aminopropyl CPG500, Solid support 848c. (FIG. 13)

Following the method described in Example 165, solid support 838c (10.00 g) was alkylated with methyl iodide (0.35 M in acetonitrile, 45.188 mL) and N,N-diisopropylethylamine (0.561 g). The solid support was filtered off, washed with 025M DIPEA-HCl in acetonitrile (58 mL) and with acetonitrile (5×50 mL), and dried to give the loading of 72.3 μmol/g.

### Example 191

### N-[(rel-(1R,3-endo,5S,6S,7R)-7-(bis(4-methoxyphenyl)(phenyl)methoxy)-6-acetoxy-8,8-dimethyl-8-azoniabicyclo[3.2.1]octane-3-yl)]succinamic acid, covalently bound to aminopropyl CPG500, Solid support 761c. (FIG. 12)

Following the method described in Example 165, solid support 751c (10.00 g) was alkylated with methyl iodide (0.35 M in acetonitrile, 46.750 mL) and N,N-diisopropylethylamine (0.580 g). The solid support was filtered off, washed with 025M DIPEA-HCl in acetonitrile (60 mL) and with acetonitrile (5×50 mL), and dried to give the loading of 74.8 μmol/g.

### Example 192

### N-[(rel-(1R,3-endo,5S,6S,7R)-7-(bis(4-methoxyphenyl)(phenyl)methoxy)-6-acetoxy-8-methyl-8-(i-propyl)-8-azoniabicyclo[3.2.1]octane-3-yl)]succinamic acid, covalently bound to aminopropyl CPG1500, Solid support 763c. (FIG. 12)

Following the method described in Example 165, solid support 753c (10.00 g) was alkylated with methyl iodide (0.35 M in acetonitrile, 20.000 mL) and N,N-diisopropylethylamine (0.248 g). The solid support was filtered off, washed with 025M DIPEA-HCl in acetonitrile (26 mL) and with acetonitrile (5×50 mL), and dried to give the loading of 32.0 μmol/g.

### Example 193

### N-[(rel-(1R,3-endo,5S,6S,7R)-7-(tris(4-methoxyphenyl)methoxy)-6-acetoxy-8,8-dimethyl-8-azoniabicyclo[3.2.1]octane-3-yl)]succinamic acid, covalently bound to aminopropyl CPG1500, Solid support 861c. (FIG. 13)

Following the method described in Example 165, solid support 851c (10.00 g) was alkylated with methyl iodide (0.35 M in acetonitrile, 20.563 mL) and N,N-diisopropylethylamine (0.255 g). The solid support was filtered off, washed with 025M DIPEA-HCl in acetonitrile (26 mL) and with acetonitrile (5×50 mL), and dried to give the loading of 32.9 μmol/g.

### Example 194

### N-[(rel-(1R,3-endo,5S,6S,7R)-7-(tris(4-methoxyphenyl)methoxy)-6-acetoxy-8,8-dimethyl-8-azoniabicyclo[3.2.1]octane-3-yl)]succinamic acid, covalently bound to hydroxypropyl CPG500, Solid support 861c. (FIG. 13)

Following the method described in Example 165, solid support 859c (10.00 g) was alkylated with methyl iodide (0.35 M in acetonitrile, 46.625 mL) and N,N-diisopropylethylamine (0.579 g). The solid support was filtered off, washed with 025M DIPEA-HCl in acetonitrile (60 mL) and with acetonitrile (5×50 mL), and dried to give the loading of 74.6 μmol/g.

### Example 195

### N-methyl-N-[(rel-(1R,3-endo,5S,6S,7R)-7-(tris(4-methoxyphenyl)methoxy)-6-acetoxy-8,8-dimethyl-8-azoniabicyclo[3.2.1]octane-3-yl)]succinamic acid, covalently bound to aminopropyl CPG500, Solid support 862c. (FIG. 13)

Following the method described in Example 165, solid support 852c (10.00 g) was alkylated with methyl iodide (0.35 M in acetonitrile, 46.000 mL) and N,N-diisopropylethylamine (0.571 g). The solid support was filtered off, washed with 025M DIPEA-HCl in acetonitrile (59 mL) and with acetonitrile (5×50 mL), and dried.

### Example 196

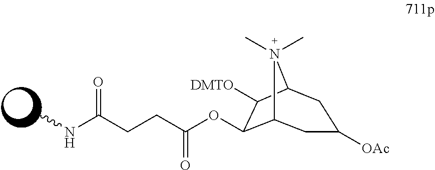

### (rel-(1R,3-endo,5S,6S,7R)-7-[(Bis(4-methoxyphenyl)(phenyl)methoxy]-3-acetoxy-8,8-dimethyl-azoniabicyclo[3.2.1]octane-6-yl) hemisuccinate, covalently bound to aminomethyl MPPS, 711p. (FIG. 12)

Methyl iodide (1 M in MeCN, 68 mL) and N,N-diisopropylethylamine (2.2 g) were added to a solid support 701p (10 g). The resulting suspension was shaken for 12 h. The solid support was filtered off, washed on the filter with 0.25M DIPEA-HCl in acetonitrile (135 mL) and then with acetonitrile (5×50 mL) and dried in vacuo. The loading of the finished solid support 701c (341.0 μmol/g) was determined, as applicable, by the standard di- or trimethoxytrityl assay as disclosed in Guzaev, A. P. and Pon, R. T. Curr. Protoc. Nucleic Acid Chem. 2013, 52, pp. 3.2.1-3.2.23.

### Example 197

### (rel-(1R,3-endo,5S,6S,7R)-7-(bis(4-methoxyphenyl)(phenyl)methoxy)-3-pivaloyloxy-8-(i-propyl)-8-azoniabicyclo[3.2.1]octane-6-yl) hemisuccinate, covalently bound to aminomethyl MPPS, Solid support 712p. (FIG. 12)

Following the method described in Example 196, solid support 703p (10.0 g) was alkylated with methyl iodide (1 M in acetonitrile, 67.6 mL) and N,N-diisopropylethylamine (2.184 g). The solid support was filtered off, washed with 0.25M DIPEA-HCl in acetonitrile (135 mL) and with acetonitrile (5×50 mL), and dried to give the loading of 338 μmol/g. EXAMPLE 198

### (rel-(1R,3-endo,5S,6S,7R)-7-(bis(4-methoxyphenyl)(phenyl)methoxy)-3-isobutyryloxy-8-benzyl-8-azoniabicyclo[3.2.1]octane-6-yl) hemisuccinate, covalently bound to aminomethyl MPPS, Solid support 713p. (FIG. 12)

Following the method described in Example 196, solid support 705p (10.0 g) was alkylated with methyl iodide (1 M in acetonitrile, 67.29 mL) and N,N-diisopropylethylamine (2.174 g). The solid support was filtered off, washed with 0.25M DIPEA-HCl in acetonitrile (135 mL) and with acetonitrile (5×50 mL), and dried to give the loading of 336 μmol/g.

### Example 199

### (rel-(1R,3-endo,5S,6S,7R)-7-(tris(4-methoxyphenyl)methoxy)-3-acetoxy-8,8-dimethyl-8-azoniabicyclo[3.2.1]octane-6-yl) hemisuccinate, covalently bound to aminomethyl MPPS, Solid support 811p. (FIG. 13)

Following the method described in Example 196, solid support 801p (10.0 g) was alkylated with methyl iodide (1 M in acetonitrile, 67.19 mL) and N,N-diisopropylethylamine (2.171 g). The solid support was filtered off, washed with 0.25M DIPEA-HCl in acetonitrile (134 mL) and with acetonitrile (5×50 mL), and dried to give the loading of 336 μmol/g.

### Example 200

### (rel-(1R,3-endo,5S,6S,7R)-7-(tris(4-methoxyphenyl)methoxy)-3-pivaloyloxy-8-methyl-8-(i-propyl)-8-azoniabicyclo[3.2.1]octane-6-yl) hemisuccinate, covalently bound to aminomethyl MPPS, Solid support 812p

Following the method described in Example 196, solid support 803p (10.0 g) was alkylated with methyl iodide (1 M in acetonitrile, 64.02 mL) and N,N-diisopropylethylamine (2.069 g). The solid support was filtered off, washed with 0.25M DIPEA-HCl in acetonitrile (128 mL) and with acetonitrile (5×50 mL), and dried to give the loading of 320 μmol/g.

### Example 201

### (rel-(1R,3-endo,5S,6S,7R)-7-(tris(4-methoxyphenyl)methoxy)-3-isobutyryloxy-8-methyl-8-benzyl-8-azoniabicyclo[3.2.1]octane-6-yl) hemisuccinate, covalently bound to aminomethyl MPPS, Solid support 813p. (FIG. 13)

Following the method described in Example 196, solid support 805p (10.0 g) was alkylated with methyl iodide (1 M in acetonitrile, 66.52 mL) and N,N-diisopropylethylamine (2.150 g). The solid support was filtered off, washed with 0.25M DIPEA-HCl in acetonitrile (133 mL) and with acetonitrile (5×50 mL), and dried to give the loading of 333 μmol/g.

### Example 202

### (rel-(1R,3-endo,5S,6S,7R)-7-(tris(4-methoxyphenyl)methoxy)-3-[(t-butyl)dimethylsilyloxy]-8-methyl-8-azoniabicyclo[3.2.1]octane-6-yl) hemisuccinate, covalently bound to aminomethyl MPPS, Solid support 817p. (FIG. 13)

Following the method described in Example 196, solid support 816p (10.0 g) was alkylated with methyl iodide (1 M in acetonitrile, 65.26 mL) and N,N-diisopropylethylamine (2.109 g). The solid support was filtered off, washed with 0.25M DIPEA-HCl in acetonitrile (131 mL) and with acetonitrile (5×50 mL), and dried to give the loading of 326 μmol/g.

### Example 203

### (rel-(1R,3-endo,5S,6S,7R)-7-(tris(4-methoxyphenyl)methoxy)-3-[(t-butyl) diphenylsilyloxy]-8-methyl-8-azoniabicyclo[3.2.1]octane-6-yl) hemisuccinate, covalently bound to aminomethyl MPPS, Solid support 819p. (FIG. 13)

Following the method described in Example 196, solid support 818p (10.0 g) was alkylated with methyl iodide (1 M in acetonitrile, 66.7 mL) and N,N-diisopropylethylamine (2.155 g). The solid support was filtered off, washed with 0.25M DIPEA-HCl in acetonitrile (133 mL) and with acetonitrile (5×50 mL), and dried to give the loading of 334 μmol/g.

### Example 204

### (rel-(1R,3-endo,5S,6S,7R)-7-(tris(4-methoxyphenyl)methoxy)-8,8-dimethyl-8-azoniaspiro[bicyclo[3.2.1]octane-3,2′-[1,3]dioxolane]-6-yl) hemisuccinate, covalently bound to aminomethyl MPPS, Solid support 825p

Following the method described in Example 196, solid support 821p (10.0 g) was alkylated with methyl iodide (1 M in acetonitrile, 66.41 mL) and N,N-diisopropylethylamine (2.146 g). The solid support was filtered off, washed with 0.25M DIPEA-HCl in acetonitrile (133 mL) and with acetonitrile (5×50 mL), and dried to give the loading of 324 μmol/g.

### Example 205

### (rel-(1R,3-endo,5S,6S,7R)-7-(tris(4-methoxyphenyl)methoxy)-5′,5′,8-trimethyl-8-azoniaspiro[bicyclo[3.2.1]octane-3,2′-[1,3]dioxane]-6-yl) hemisuccinate, covalently bound to aminomethyl MPPS, Solid support 826p. (FIG. 13)

Following the method described in Example 196, solid support 822p (10.0 g) was alkylated with methyl iodide (1 M in acetonitrile, 64.88 mL) and N,N-diisopropylethylamine (2.096 g). The solid support was filtered off, washed with 0.25M DIPEA-HCl in acetonitrile (130 mL) and with acetonitrile (5×50 mL), and dried to give the loading of 341 μmol/g.

### Example 206

### (rel-(1R,3-endo,5S,6S,7R)-7-(tris(4-methoxyphenyl)methoxy)-8-methyl-8-(i-propyl)-8-azoniaspiro[bicyclo[3.2.1]octane-3,2′-[1,3]dioxolane]-6-yl) hemisuccinate, covalently bound to aminomethyl MPPS, Solid support 827p. (FIG. 13)

Following the method described in Example 196, solid support 823p (10.0 g) was alkylated with methyl iodide (1 M in acetonitrile, 64.78 mL) and N,N-diisopropylethylamine (2.093 g). The solid support was filtered off, washed with 0.25M DIPEA-HCl in acetonitrile (130 mL) and with acetonitrile (5×50 mL), and dried to give the loading of 339 μmol/g.

### Example 207

### (rel-(1R,3-endo,5S,6S,7R)-7-(tris(4-methoxyphenyl)methoxy)-5′,5′,8-trimethyl-8-azoniaspiro[bicyclo[3.2.1]octane-3,2′-[1,3]dioxane]-6-yl) hemisuccinate, covalently bound to aminomethyl MPPS, Solid support 828p. (FIG. 13)

Following the method described in Example 196, solid support 824p (10.0 g) was alkylated with methyl iodide (1 M in acetonitrile, 64.87 mL) and N,N-diisopropylethylamine (2.096 g). The solid support was filtered off, washed with 0.25M DIPEA-HCl in acetonitrile (130 mL) and with acetonitrile (5×50 mL), and dried to give the loading of 328 μmol/g.

### Example 208

### (rel-(1R,3-endo,5S,6S,7R)-7-(bis(4-methoxyphenyl)(phenyl)methoxy)-6-acetoxy-8,8-dimethyl-8-azoniabicyclo[3.2.1]octane-3-yl) hemisuccinate, covalently bound to aminomethyl MPPS, Solid support 741p. (FIG. 12)

Following the method described in Example 196, solid support 731p (10.0 g) was alkylated with methyl iodide (1 M in acetonitrile, 68.63 mL) and N,N-diisopropylethylamine (2.218 g). The solid support was filtered off, washed with 0.25M DIPEA-HCl in acetonitrile (137 mL) and with acetonitrile (5×50 mL), and dried to give the loading of 328 μmol/g.

### Example 209

### (rel-(1R,3-endo,5S,6S,7R)-7-(bis(4-methoxyphenyl)(phenyl)methoxy)-6-acetoxy-8,8-diethyl-8-azoniabicyclo[3.2.1]octane-3-yl) hemisuccinate, covalently bound to aminomethyl MPPS, Solid support 742p. (FIG. 12)

Following the method described in Example 196, solid support 732p (10.0 g) was alkylated with ethyl iodide (1 M in acetonitrile, 65.4 mL) and N,N-diisopropylethylamine (2.113 g). The solid support was filtered off, washed with 0.25M DIPEA-HCl in acetonitrile (131 mL) and with acetonitrile (5×50 mL), and dried to give the loading of 334 μmol/g.

### Example 210

### (rel-(1R,3-endo,5S,6S,7R)-7-(bis(4-methoxyphenyl)(phenyl)methoxy)-6-acetoxy-8-methyl-8-(i-propyl)-8-azoniabicyclo[3.2.1]octane-3-yl) hemisuccinate, covalently bound to aminomethyl MPPS, Solid support 743p. (FIG. 12)

Following the method described in Example 196, solid support 733p (10.0 g) was alkylated with methyl iodide (1 M in acetonitrile, 65.25 mL) and N,N-diisopropylethylamine (2.109 g). The solid support was filtered off, washed with 0.25M DIPEA-HCl in acetonitrile (131 mL) and with acetonitrile (5×50 mL), and dried to give the loading of 331 μmol/g.

### Example 211

### (rel-(1R,3-endo,5S,6S,7R)-7-(bis(4-methoxyphenyl)(phenyl)methoxy)-6-acetoxy-8-methyl-8-benzyl-8-azoniabicyclo[3.2.1]octane-3-yl) hemisuccinate, covalently bound to aminomethyl MPPS, Solid support 744p. (FIG. 12)

Following the method described in Example 196, solid support 734p (10.0 g) was alkylated with methyl iodide (1 M in acetonitrile, 66.11 mL) and N,N-diisopropylethylamine (2.136 g). The solid support was filtered off, washed with 0.25M DIPEA-HCl in acetonitrile (132 mL) and with acetonitrile (5×50 mL), and dried to give the loading of 334 μmol/g.

### Example 212

### (rel-(1R,3-endo,5S,6S,7R)-7-(bis(4-methoxyphenyl)(phenyl)methoxy)-6-acetoxy-8,8-dimethyl-8-azoniabicyclo[3.2.1]octane-3-yl) diglycolate, covalently bound to aminomethyl MPPS, Solid support 745p. (FIG. 12)

Following the method described in Example 196, solid support 735p (10.0 g) was alkylated with methyl iodide (1 M in acetonitrile, 67.09 mL) and N,N-diisopropylethylamine (2.168 g). The solid support was filtered off, washed with 0.25M DIPEA-HCl in acetonitrile (134 mL) and with acetonitrile (5×50 mL), and dried to give the loading of 327 μmol/g.

### Example 213

### (rel-(1R,3-endo,5S,6S,7R)-7-(bis(4-methoxyphenyl)(phenyl)methoxy)-6-acetoxy-2,4,8,8-tetramethyl-8-azoniabicyclo[3.2.1]octane-3-yl) hemisuccinate, covalently bound to aminomethyl MPPS, Solid support 747p. (FIG. 12)

Following the method described in Example 196, solid support 737p (10.0 g) was alkylated with methyl iodide (1 M in acetonitrile, 65.28 mL) and N,N-diisopropylethylamine (2.109 g). The solid support was filtered off, washed with 0.25M DIPEA-HCl in acetonitrile (131 mL) and with acetonitrile (5×50 mL), and dried to give the loading of 345 μmol/g.

### Example 214

### (rel-(1R,3-endo,5S,6S,7R)-7-(bis(4-methoxyphenyl)(phenyl)methoxy)-6-acetoxy-2,2,4,4,8,8-hexamethyl-8-azoniabicyclo[3.2.1]octane-3-yl) hemisuccinate, covalently bound to aminomethyl MPPS, Solid support 748p. (FIG. 12)

Following the method described in Example 196, solid support 738p (10.0 g) was alkylated with methyl iodide (1 M in acetonitrile, 66.26 mL) and N,N-diisopropylethylamine (2.141 g). The solid support was filtered off, washed with 0.25M DIPEA-HCl in acetonitrile (133 mL) and with acetonitrile (5×50 mL), and dried to give the loading of 345 μmol/g.

### Example 215

### (rel-(1R,3-endo,5S,6S,7R)-7-(tris(4-methoxyphenyl)methoxy)-6-acetoxy-8,8-dimethyl-8-azoniabicyclo[3.2.1]octane-3-yl) hemisuccinate, covalently bound to aminomethyl MPPS, Solid support 841p. (FIG. 13)

Following the method described in Example 196, solid support 831p (10.0 g) was alkylated with methyl iodide (1 M in acetonitrile, 67.34 mL) and N,N-diisopropylethylamine (2.176 g). The solid support was filtered off, washed with 0.25M DIPEA-HCl in acetonitrile (135 mL) and with acetonitrile (5×50 mL), and dried to give the loading of 342 μmol/g.

### Example 216

### (rel-(1R,3-endo,5S,6S,7R)-7-(tris(4-methoxyphenyl)methoxy)-6-acetoxy-8,8-diethyl-8-azoniabicyclo[3.2.1]octane-3-yl) hemisuccinate, covalently bound to aminomethyl MPPS, Solid support 842p. (FIG. 13)

Following the method described in Example 196, solid support 832p (10.0 g) was alkylated with ethyl iodide (1 M in acetonitrile, 68.11 mL) and N,N-diisopropylethylamine (2.201 g). The solid support was filtered off, washed with 0.25M DIPEA-HCl in acetonitrile (136 mL) and with acetonitrile (5×50 mL), and dried to give the loading of 342 μmol/g.

### Example 217

### (rel-(1R,3-endo,5S,6S,7R)-7-(tris(4-methoxyphenyl)methoxy)-6-acetoxy-8-methyl-8-(i-propyl)-8-azoniabicyclo[3.2.1]octane-3-yl) hemisuccinate, covalently bound to aminomethyl MPPS, Solid support 843p. (FIG. 13)

Following the method described in Example 196, solid support 833p (10.0 g) was alkylated with methyl iodide (1 M in acetonitrile, 67.7 mL) and N,N-diisopropylethylamine (2.187 g). The solid support was filtered off, washed with 0.25M DIPEA-HCl in acetonitrile (135 mL) and with acetonitrile (5×50 mL), and dried to give the loading of 343 μmol/g.

### Example 218

### (rel-(1R,3-endo,5S,6S,7R)-7-(tris(4-methoxyphenyl)methoxy)-6-acetoxy-8-methyl-8-benzyl-8-azoniabicyclo[3.2.1]octane-3-yl) hemisuccinate, covalently bound to aminomethyl MPPS, Solid support 844p. (FIG. 13)

Following the method described in Example 196, solid support 834p (10.0 g) was alkylated with methyl iodide (1 M in acetonitrile, 68.53 mL) and N,N-diisopropylethylamine (2.214 g). The solid support was filtered off, washed with 0.25M DIPEA-HCl in acetonitrile (137 mL) and with acetonitrile (5×50 mL), and dried to give the loading of 343 μmol/g.

### Example 219

### (rel-(1R,3-endo,5S,6S,7R)-7-(tris(4-methoxyphenyl)methoxy)-6-acetoxy-8,8-dimethyl-8-azoniabicyclo[3.2.1]octane-3-yl) [2-[4-(carboxymethoxy) phenoxy]acetate], covalently bound to aminomethyl MPPS, Solid support 846p. (FIG. 13)

Following the method described in Example 196, solid support 836p (10.0 g) was alkylated with methyl iodide (1 M in acetonitrile, 68.57 mL) and N,N-diisopropylethylamine (2.216 g). The solid support was filtered off, washed with 0.25M DIPEA-HCl in acetonitrile (137 mL) and with acetonitrile (5×50 mL), and dried to give the loading of 344 μmol/g.

### Example 220

### (rel-(1R,3-endo,5S,6S,7R)-7-(tris(4-methoxyphenyl)methoxy)-6-acetoxy-2,4,8,8-tetramethyl-8-azoniabicyclo[3.2.1]octane-3-yl) hemisuccinate, covalently bound to aminomethyl MPPS, Solid support 847p. (FIG. 13)

Following the method described in Example 196, solid support 837p (10.0 g) was alkylated with methyl iodide (1 M in acetonitrile, 64.42 mL) and N,N-diisopropylethylamine (2.082 g). The solid support was filtered off, washed with 0.25M DIPEA-HCl in acetonitrile (129 mL) and with acetonitrile (5×50 mL), and dried to give the loading of 345 μmol/g.

### Example 221

### (rel-(1R,3-endo,5S,6S,7R)-7-(tris(4-methoxyphenyl)methoxy)-6-acetoxy-2,2,4,4,8,8-hexamethyl-8-azoniabicyclo[3.2.1]octane-3-yl) hemisuccinate, covalently bound to aminomethyl MPPS, Solid support 848p. (FIG. 13)

Following the method described in Example 196, solid support 838p (10.0 g) was alkylated with methyl iodide (1 M in acetonitrile, 68.2 mL) and N,N-diisopropylethylamine (2.204 g). The solid support was filtered off, washed with 0.25M DIPEA-HCl in acetonitrile (136 mL) and with acetonitrile (5×50 mL), and dried to give the loading of 346 μmol/g.

### Example 222

### N-[(rel-(1R,3-endo,5S,6S,7R)-7-(bis(4-methoxyphenyl)(phenyl)methoxy)-6-acetoxy-8,8-dimethyl-8-azoniabicyclo[3.2.1]octane-3-yl)]succinamic acid, covalently bound to aminomethyl MPPS, Solid support 751p. (FIG. 10)

Following the method described in Example 196, solid support 751p (10.0 g) was alkylated with methyl iodide (1 M in acetonitrile, 69.24 mL) and N,N-diisopropylethylamine (2.237 g). The solid support was filtered off, washed with 0.25M DIPEA-HCl in acetonitrile (138 mL) and with acetonitrile (5×50 mL), and dried to give the loading of 346 μmol/g.

### Example 223

### N-[(rel-(1R,3-endo,5S,6S,7R)-7-(bis(4-methoxyphenyl)(phenyl)methoxy)-6-acetoxy-8-methyl-8-(i-propyl)-8-azoniabicyclo[3.2.1]octane-3-yl)]succinamic acid, covalently bound to aminomethyl MPPS, Solid support 753p. (FIG. 10)

Following the method described in Example 196, solid support 753p (10.0 g) was alkylated with methyl iodide (1 M in acetonitrile, 69.47 mL) and N,N-diisopropylethylamine (2.245 g). The solid support was filtered off, washed with 0.25M DIPEA-HCl in acetonitrile (139 mL) and with acetonitrile (5×50 mL), and dried to give the loading of 347 μmol/g.

### Example 224

### N-[(rel-(1R,3-endo,5S,6S,7R)-7-(tris(4-methoxyphenyl)methoxy)-6-acetoxy-8,8-dimethyl-8-azoniabicyclo[3.2.1]octane-3-yl)]succinamic acid, covalently bound to aminomethyl MPPS, Solid support 851p. (FIG. 11)

Following the method described in Example 196, solid support 851p (10.0 g) was alkylated with methyl iodide (1 M in acetonitrile, 70.39 mL) and N,N-diisopropylethylamine (2.275 g). The solid support was filtered off, washed with 0.25M DIPEA-HCl in acetonitrile (141 mL) and with acetonitrile (5×50 mL), and dried to give the loading of 352 μmol/g.

### Example 225

### N-[(rel-(1R,3-endo,5S,6S,7R)-7-(tris(4-methoxyphenyl)methoxy)-6-acetoxy-8,8-dimethyl-8-azoniabicyclo[3.2.1]octane-3-yl)]succinamic acid, covalently bound to hydroxymethyl MPPS, Solid support 859p. (FIG. 11)

Following the method described in Example 196, solid support 851p (10.0 g) was alkylated with methyl iodide (1 M in acetonitrile, 70.39 mL) and N,N-diisopropylethylamine (2.275 g). The solid support was filtered off, washed with 0.25M DIPEA-HCl in acetonitrile (141 mL) and with acetonitrile (5×50 mL), and dried to give the loading of 352 μmol/g.

### Example 226

### N-methyl-N-[(rel-(1R,3-endo,5S,6S,7R)-7-(tris(4-methoxyphenyl)methoxy)-6-acetoxy-8,8-dimethyl-8-azoniabicyclo[3.2.1]octane-3-yl)]succinamic acid, covalently bound to aminomethyl MPPS, Solid support 852p. (FIG. 11)

Following the method described in Example 196, solid support 852p (10.0 g) was alkylated with methyl iodide (1 M in acetonitrile, 70.51 mL) and N,N-diisopropylethylamine (2.278 g). The solid support was filtered off, washed with 0.25M DIPEA-HCl in acetonitrile (141 mL) and with acetonitrile (5×50 mL), and dried to give the loading of 353 μmol/g.

### Example 227

### (rel-(1R,3-endo,5S,6S,7R)—N-[7-[Tris(4-methoxyphenyl)methoxy]-6-acetoxy-8,8-dimethyl-azoniabicyclo[3.2.1]octane-6-yl)]carbamic acid, covalently bound to aminopropyl CPG1000, 864c. (FIG. 13)

Following the method described in Example 196, solid support 854c (10.0 g) was alkylated with methyl iodide (1 M in acetonitrile, 27.0 mL) and N,N-diisopropylethylamine (0.336 g). The solid support was filtered off, washed with 0.25M DIPEA-HCl in acetonitrile (40 mL) and with acetonitrile (5×50 mL), and dried to give the loading of 43.3 μmol/g.

### Example 228

### [6-[methyl[(rel-(1R,3-endo,5S,6S,7R)—N-[7-[Tris(4-methoxyphenyl)methoxy]-6-acetoxy-8,8-dimethyl-azoniabicyclo[3.2.1]octane-6-yl)]amino]-6-oxohexyl]carbamic acid, covalently bound to aminopropyl CPG500, 865c. (FIG. 13)

Following the method described in Example 196, solid support 855c (10.0 g) was alkylated with methyl iodide (1 M in acetonitrile, 45.7 mL) and N,N-diisopropylethylamine (0.567 g). The solid support was filtered off, washed with 0.25M DIPEA-HCl in acetonitrile (58 mL) and with acetonitrile (5×50 mL), and dried to give the loading of 73.1 μmol/g.

### Example 229

### [6-[methyl[(rel-(1R,3-endo,5S,6S,7R)—N-[7-[Tris(4-methoxyphenyl)methoxy]-6-acetoxy-8,8-dimethyl-azoniabicyclo[3.2.1]octane-3-yl)amino]-6-oxohexyl]carbamic acid, covalently bound to hydroxypropyl CPG500, 866c. (FIG. 13)

Following the method described in Example 196, solid support 856c (10.0 g) was alkylated with methyl iodide (1 M in acetonitrile, 43 mL) and N,N-diisopropylethylamine (0.534 g). The solid support was filtered off, washed with 0.25M DIPEA-HCl in acetonitrile (55 mL) and with acetonitrile (5×50 mL), and dried to give the loading of 68.8 μmol/g.

### Example 230

### [6-[3-[(rel-(1R,3-endo,5S,6S,7R)—N-[7-[Tris(4-methoxyphenyl)methoxy]-6-acetoxy-8,8-dimethyl-azoniabicyclo[3.2.1]octane-3-yl) ureido]hexyl]carbamic acid, covalently bound to hydroxypropyl CPG500, 868c. (FIG. 13)

Following the method described in Example 196, solid support 858c (10.0 g) was alkylated with methyl iodide (1 M in acetonitrile, 42.1 mL) and N,N-diisopropylethylamine (0.523 g). The solid support was filtered off, washed with 0.25M DIPEA-HCl in acetonitrile (54 mL) and with acetonitrile (5×50 mL), and dried to give the loading of 67.4 μmol/g.

### Example 231

### (rel-(1R,3-endo,5S,6S,7R)—N-[7-[Tris(4-methoxyphenyl)methoxy]-6-acetoxy-8,8-dimethyl-azoniabicyclo[3.2.1]octane-6-yl)]carbamic acid, covalently bound to hydroxymethyl MPPS, 864p. (FIG. 13)

Following the method described in Example 196, solid support 854p (5.0 g) was alkylated with methyl iodide (1 M in acetonitrile, 35.0 mL) and N,N-diisopropylethylamine (2.27 g). The solid support was filtered off, washed with 0.25M DIPEA-HCl in acetonitrile (35 mL) and with acetonitrile (5×50 mL), and dried to give the loading of 354 μmol/g.

### Example 232

### [6-[methyl[(rel-(1R,3-endo,5S,6S,7R)—N-[7-[Tris(4-methoxyphenyl)methoxy]-6-acetoxy-8,8-dimethyl-azoniabicyclo[3.2.1]octane-6-yl)]amino]-6-oxohexyl]carbamic acid, covalently bound to hydroxymethyl MPPS, 865p. (FIG. 13)

Following the method described in Example 196, solid support 855p (10.0 g) was alkylated with methyl iodide (1 M in acetonitrile, 45.7 mL) and N,N-diisopropylethylamine (0.567 g). The solid support was filtered off, washed with 0.25M DIPEA-HCl in acetonitrile (38 mL) and with acetonitrile (5×50 mL), and dried to give the loading of 346 μmol/g.

### Example 233

### [6-[methyl[(rel-(1R,3-endo,5S,6S,7R)—N-[7-[Tris(4-methoxyphenyl)methoxy]-6-acetoxy-8,8-dimethyl-azoniabicyclo[3.2.1]octane-3-yl)amino]-6-oxohexyl]carbamic acid, covalently bound to hydroxymethyl MPPS, 866p. (FIG. 13)

Following the method described in Example 196, solid support 856p (5.0 g) was alkylated with methyl iodide (1 M in acetonitrile, 35.4 mL) and N,N-diisopropylethylamine (1.15 g). The solid support was filtered off, washed with 0.25M DIPEA-HCl in acetonitrile (35 mL) and with acetonitrile (5×50 mL), and dried to give the loading of 322 μmol/g.

### Example 234

### [6-[3-[(rel-(1R,3-endo,5S,6S,7R)—N-[7-[Tris(4-methoxyphenyl)methoxy]-6-acetoxy-8,8-dimethyl-azoniabicyclo[3.2.1]octane-3-yl) ureido]hexyl]carbamic acid, covalently bound to hydroxymethyl MPPS, 868p. (FIG. 13)

Following the method described in Example 196, solid support 858p (5.0 g) was alkylated with methyl iodide (1 M in acetonitrile, 35.5 mL) and N,N-diisopropylethylamine (1.14 g). The solid support was filtered off, washed with 0.25M DIPEA-HCl in acetonitrile (54 mL) and with acetonitrile (5×50 mL), and dried to give the loading of 325 μmol/g.

### Example 235

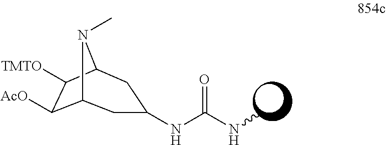

### (rel-(1R,3-endo,5S,6S,7R)—N-[7-[Tris(4-methoxyphenyl)methoxy]-6-acetoxy-8-methyl-azabicyclo[3.2.1]octane-6-yl)]carbamic acid, covalently bound to aminopropyl CPG1000, 854c

A solution of 1,1-carbonyldiimidazole (97 mg, 0.6 mmol) in DMF (2 mL) was added dropwise to compound 451 (290 mg, 0.57 mmol) in acetonitrile (4 mL) plus DIPEA (72 g, 0.57 mmol) under stirring in an ice bath. The mixture was stirred overnight and was transferred to a suspension of aminopropyl CPG1000 (11.5 g) in anhydrous acetonitrile (40 mL) plus DIPEA (176 mg). The suspension was shaken overnight, and the completion of the loading reaction was verified by the standard trimethoxytrityl assay as disclosed in Guzaev, A. P. and Pon, R. T. Curr. Protoc. Nucleic Acid Chem. 2013, 52, pp. 3.2.1-3.2.23. N-methylimidazole (1.5 mL) was added followed by acetic anhydride (1.5 mL). The mixture was shaken for another 6 h, which was required for the completion of acetylation of the hydroxy group at the position 6 of tropane ring. The solid support was filtered off, washed on filter with acetonitrile (5×50 mL) and dried in vacuo to give the loading of 43.3 μmol/g.

Universal solid supports 854p, 855c, 855p, 865c, 856p, 858c, and 858p (FIG. 11) were prepared following the same method to obtain the loadings of 354, 73.1, 346, 68.8, 322, 67.4, and 325 μmol/g, respectively.

### Example 236

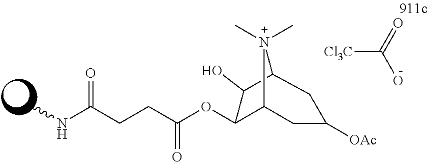

### (rel-(1R,3-endo,5S,6S,7R)-7-hydroxy-3-acetoxy-8,8-dimethyl-azoniabicyclo[3.2.1]octane-6-yl) hemisuccinate, covalently bound to aminopropyl CPG1000, 911c

Detritylation agent comprising an anhydrous solution of trichloroacetic acid in toluene (5%, 60 mL) was passed through a column containing solid support 811c (10 g) at a rate of 6 mL/min. The column was flushed with a flow of nitrogen and was washed with a solution of pyridine in acetonitrile (5%, 40 mL) at a flow rate of 10 to 20 mL/min. Optionally, after washing with 5% pyridine in acetonitrile, the washing cycle may include an additional wash with 0.25 M DIPEA-HCl in acetonitrile (5 mL/g) to replace trichloroacetate counter ion with chloride. Finally, the support was washed with acetonitrile (5×50 mL), flushed with a flow of nitrogen, and dried in vacuo. The loading of the finished solid support 911c (69.0 μmol/g) was determined by coupling DMT-T phosphoramidite to a 1 μmol sample of the support followed by the standard dimethoxytrityl assay as disclosed in Guzaev, A. P. and Pon, R. T. Curr. Protoc. Nucleic Acid Chem. 2013, 52, pp. 3.2.1-3.2.23.

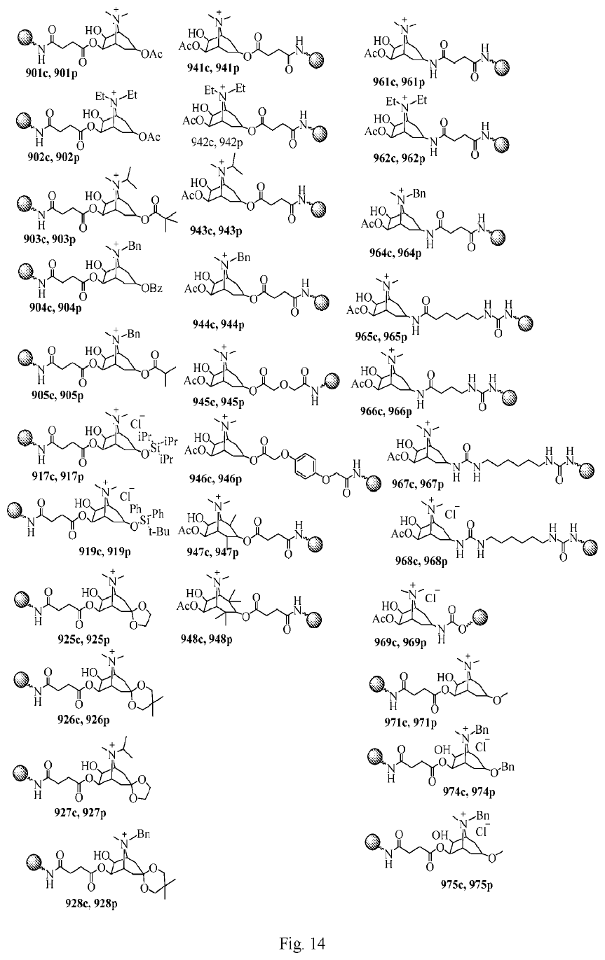

Deprotected solid supports (FIG. 14) 911c-913c, 917c, 919c, 925c, 927c, 929c, 942c-945c, 946c-948c, 961c-966c, 968c, 969c, 971c, 974c, and 975c were obtained following the procedure disclosed above.

### Example 237

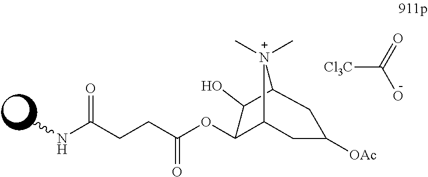

### (rel-(1R,3-endo,5S,6S,7R)-7-hydroxy-3-acetoxy-8,8-dimethyl-azoniabicyclo[3.2.1]octane-6-yl) hemisuccinate, covalently bound to aminomethyl MPPS, 911p

Detritylation agent comprising an anhydrous solution of trichloroacetic acid in toluene (5%, 60 mL) was passed through a column containing solid support 811p (10 g) at a rate of 6 mL/min. The column was flushed with a flow of nitrogen and was washed with a solution of pyridine in acetonitrile (5%, 60 mL) at a flow rate of 10 to 20 mL/min. Optionally, after washing with 5% pyridine in acetonitrile, the washing cycle may include an additional wash with 0.25 M DIPEA-HCl in acetonitrile (5 mL/g) to replace trichloroacetate counter ion with chloride. Finally, the support was washed with acetonitrile (5×50 mL), flushed with a flow of nitrogen, and dried in vacuo. The loading of the finished solid support 911p (341 μmol/g) was determined by coupling DMT-T phosphoramidite to a 1 μmol sample of the support followed by the standard dimethoxytrityl assay as disclosed in Guzaev, A. P. and Pon, R. T. Curr. Protoc. Nucleic Acid Chem. 2013, 52, pp. 3.2.1-3.2.23.

Deprotected solid supports (FIG. 14) 911p-913p, 917p, 919p, 925p, 927p, 929p, 942p-945p, 946p-948p, 961p-966p, 968p, 969p, 971p, 974p, and 975p were obtained following the procedure disclosed above.

### Example 238

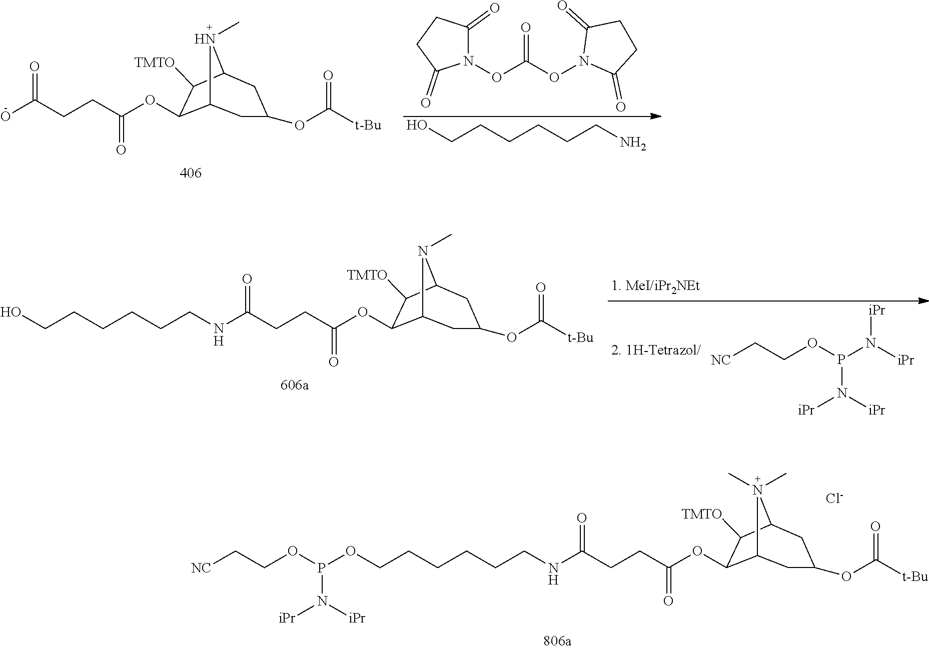

### Universal Phosphoramidite 806a

A mixture of disuccinimidyl carbonate (705 mg, 2.75 mmol) and compound 406 (2.41 g, 2.5 mmol) in anhydrous pyridine (15 mL) was stirred overnight, and 6-aminohexanol (366 mg, 3.12 mmol) was added. The mixture was stirred for 6 h, the solvent was evaporated, and the reside was distributed between dichloromethane (100 mL) and 1M NaH₂PO₄ (20 mL). The organic phase was washed with 1M NaH₂PO₄ (2×50 mL), 5% aq NaHCO₃ (2×50 mL), and with brine (30 mL). After drying over Na₂SO₄, the organic phase was evaporated and dried under vacuum to give crude compound 606a.

Crude 606a was dissolved in acetonitrile, DIPEA (1.26 g, 10 mmol) and methyl iodide (1.06 g, 7.5 mmol) were added, and the mixture was stirred for 6 h at 45° C. The solvent was evaporated, the residue was dissolved in DCM (100 mL) and the solution was washed with 5% aq NaHCO₃ (2×50 mL) and with brine (30 mL). The solution was applied on a silica gel column, and the product was isolated by column purification using a linear gradient from DCM to TEA:MeOH:DCM (5:20:75). After evaporation of collected fractions and coevaporation with acetonitrile (4×25 mL), the material was dried on an oil pump.

A mixture of the product of the previous step, 2-cyanoethyl bis(N,N-diisopropylamino) phosphite (829 mg, 2.75 mmol), and acetonitrile (5 mL) was charged with a solution of 1H-tetrazole in acetonitrile (0.45 M, 3.9 mL, 1.75 mmol), and the mixture was stirred overnight. Triethylamine (202 mg, 2 mmol) was added, the mixture was diluted with DCM (100 mL) and was washed with 5% aq NaHCO₃ (2×20 mL) and with brine (30 mL). The organic phase was dried over Na₂SO₄ and evaporated. The residue was applied on a silica gel column, and the product was isolated by column purification using a linear gradient from DCM to TEA:MeOH:DCM (5:5:90). After evaporation of collected fractions and coevaporation with acetonitrile (4×25 mL), the material was dried on an oil pump to give the desired product as a solid foam (1840 mg, 70.8%). ³¹P NMR (CD₃CN): δ 149.45, 149.6. ES MS: [M+H]⁺ 1004.7 (observed), 1005.2 (calcd.).

### Example 239

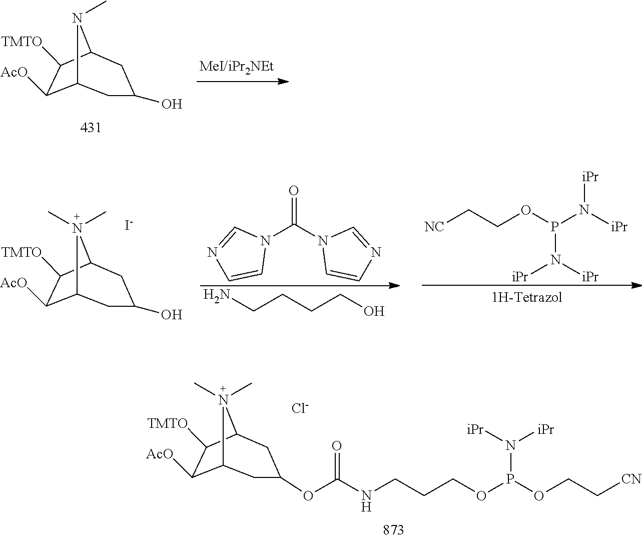

### 2-Cyanoethyl rel-(1R,5S,6S,7R)-3-[[[[7-(tris(4-methoxyphenyl)methoxy)-6-acetoxy-8,8-dimethyl-8-azoniabicyclo[3.2.1]octan-3-yl]oxy]carbonyl]amino]propyl(N,N-diisopropylamino) phosphite 873

Compound 431 (1095 mg, 2.0 mmol) was dissolved in acetonitrile, DIPEA (1.26 g, 10 mmol) and methyl iodide (1.06 g, 7.5 mmol) were added, and the mixture was stirred for 6 h at 45° C. The solvent was evaporated, acetonitrile (10 mL), N,N-carbonyldiimidazole (405 mg, 2.5 mmol), and DIPEA (378 mg, 3 mmol) were added to the residue. The mixture was stirred overnight at 45° C., cooled in ice-water bath, and 3-aminopropanol (225 mg, 3.0 mmol) in acetonitrile (3 mL) were added to the residue. The reaction was stirred for 3 h, diluted with DCM (100 mL), and washed with 1M NaH₂PO₄ (2×50 mL) and with brine (30 mL). After drying over Na₂SO₄, the organic phase was applied on a silica gel column. The product was isolated by column purification using a linear gradient from DCM to TEA:MeOH:DCM (5:25:70). After evaporation of collected fractions and coevaporation with acetonitrile (4×25 mL), the material was dried on an oil pump.

A mixture of the product of the previous step and 2-cyanoethyl bis(N,N-diisopropylamino) phosphite (663 mg, 2.2 mmol) was charged with a solution of 1H-tetrazole in acetonitrile (0.45 M, 3.1 mL, 1.4 mmol), and the mixture was stirred overnight. Triethylamine (202 mg, 2 mmol) was added, the mixture was diluted with DCM (100 mL) and was washed with 5% aq NaHCO₃ (2×20 mL) and with brine (30 mL). The organic phase was dried over Na₂SO₄ and evaporated. The residue was applied on a silica gel column, and the product was isolated by column purification using a linear gradient from DCM to TEA:MeOH:DCM (5:10:85). After evaporation of collected fractions and coevaporation with acetonitrile (4×25 mL), the material was dried on an oil pump to give the desired product as a solid foam (1480 mg, 82.3%). ³¹P NMR (CD₃CN): δ 149.12, 149.55. ES MS: [M+H]⁺ 865.5 (observed), 865.0 (calcd.).

### Example 240

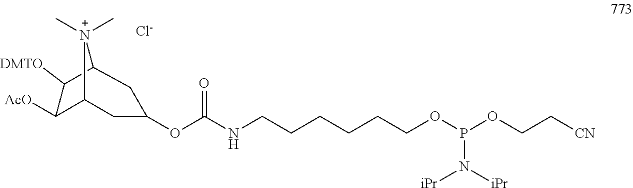

### 2-Cyanoethyl rel-(1R,5S,6S,7R)-6-[[[[7-(bis(4-methoxyphenyl)(phenyl)methoxy)-6-acetoxy-8,8-dimethyl-8-azoniabicyclo[3.2.1]octan-3-yl]oxy]carbonyl]amino]hexyl(N,N-diisopropylamino) phosphite 773

Compound 773 isolated as a white solid foam (83.1%), was synthesized from compound 331 by following the procedure described in Example 239. ³¹P NMR (CD₃CN): δ 149.22, 149.59. ES MS: [M+H]⁺ 877.7 (observed), 877.1 (calcd.).

### Example 241

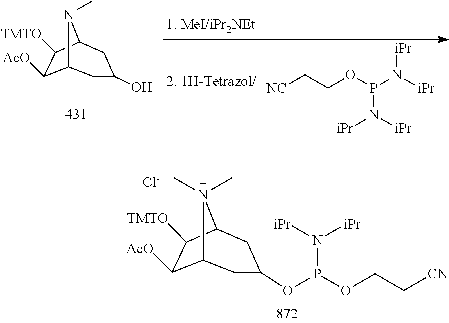

### 2-Cyanoethyl rel-(1R,5S,6S,7R)-7-(tris(4-methoxyphenyl)methoxy)-6-acetoxy-8,8-dimethyl-8-azoniabicyclo[3.2.1]octan-3-yl(N,N-diisopropylamino) phosphite, 872

Compound 431 (1369 mg, 2.5 mmol) was dissolved in acetonitrile, DIPEA (1.26 g, 10 mmol) and methyl iodide (1.06 g, 7.5 mmol) were added, and the mixture was stirred for 6 h at 45° C. The solvent was evaporated, the residue was dissolved in DCM (100 mL) and the solution was washed with 5% aq NaHCO₃ (2×50 mL) and with brine (30 mL). The solution was applied on a silica gel column, and the product was isolated by column purification using a linear gradient from DCM to TEA:MeOH:DCM (5:20:75). After evaporation of collected fractions and coevaporation with acetonitrile (4×25 mL), the material was dried on an oil pump.

A mixture of the product of the previous step, 2-cyanoethyl bis(N,N-diisopropylamino) phosphite (829 mg, 2.75 mmol), and acetonitrile (5 mL) was charged with a solution of 1H-tetrazole in acetonitrile (0.45 M, 3.9 mL, 1.75 mmol), and the mixture was stirred overnight. Triethylamine (202 mg, 2 mmol) was added, the mixture was diluted with DCM (100 mL) and was washed with 5% aq NaHCO₃ (2×20 mL) and with brine (30 mL). The organic phase was dried over Na₂SO₄ and evaporated. The residue was applied on a silica gel column, and the product was isolated by column purification using a linear gradient from DCM to TEA:MeOH:DCM (5:5:90). After evaporation of collected fractions and coevaporation with acetonitrile (4×25 mL), the material was dried on an oil pump to give the desired product as a solid foam (1714 mg, 85.9%). ³¹P NMR (CD₃CN): δ 148.53, 149.22. ES MS: [M+H]⁺ 763.3 (observed), 763.9 (calcd.).

### Example 242

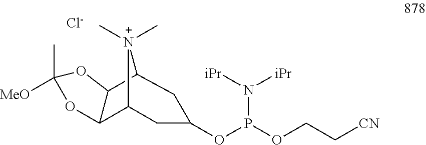 [SMILES: [Cl-] ; COC1(C)OC2C(O1)C1CC(OP(OCCC#N)N(C(C)C)C(C)C)CC2[N+]1(C)C]

### 2-Cyanoethyl 2,9-dimethyl-2-methoxy-hexahydro-6H-cyclohepta-1,3-dioxol-4,8-imin-6-yl (N,N-diisopropylamino) phosphite, 878

Compound 878 isolated as a white solid foam (83.1%), was synthesized from compound 21 by following the procedure described in Example 229. ³¹P NMR (CD₃CN): δ 149.11, 149.42. ES MS: [M+H]⁺ 446.3 (observed), 445.5 (calcd.).

### Example 243

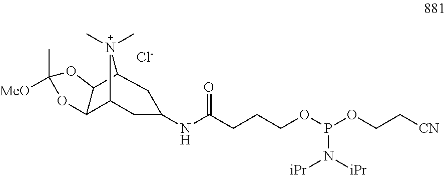 [SMILES: COC1(C)OC2C(O1)C1CC(NC(=O)CCCOP(OCCC#N)N(C(C)C)C(C)C)CC2[N+]1(C)C ; [Cl-]]

### 2-Cyanoethyl[4-[[2,9-dimethyl-2-methoxy-hexahydro-6H-cyclohepta-1,3-dioxol-4,8-imin-6-yl]amino]-4-oxobutyl (N,N-diisopropylamino) phosphite, 881

Compound 881 isolated as a white solid foam (79.2%), was synthesized from compound 880 by following the procedure described in Example 229. ³¹P NMR (CD₃CN): δ 149.34, 149.66. ES MS: [M+H]⁺ 531.1 (observed), 530.6 (calcd.).

### Example 244

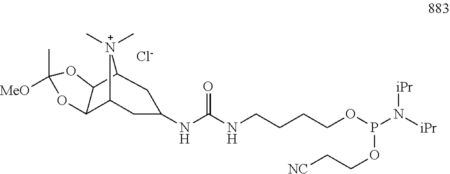 [SMILES: COC1(C)OC2C(O1)C1CC(NC(=O)NCCCCOP(OCCC#N)N(C(C)C)C(C)C)CC2[N+]1(C)C ; [Cl-]]

### Universal 2-cyanoethyl (N,N-diisopropylamino) phosphite, 883

Compound 883 isolated as a white solid foam (80.4%), was synthesized from compound 882 by following the procedure described in Example 229. ³¹P NMR (CD₃CN): δ 149.48, 149.82. ES MS: [M+H]⁺ 560.5 (observed), 559.7 (calcd.).

### Example 245

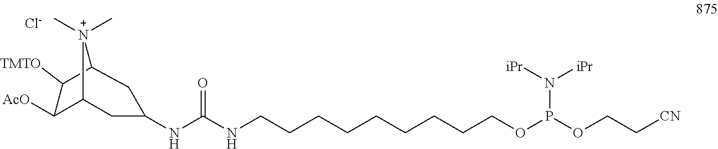

### Universal 2-cyanoethyl (N,N-diisopropylamino) phosphite, 875

Compound 875 isolated as a white solid foam (82.7%), was synthesized from compound 874 by following the procedure described in Example 229. ³¹P NMR (CD₃CN): δ 149.14, 149.54. ES MS: [M+H]⁺ 948.9 (observed), 948.2 (calcd.).

### Example 246

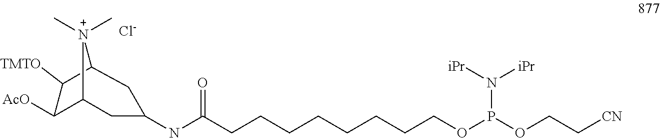

### Universal 2-cyanoethyl (N,N-diisopropylamino) phosphite, 877

Compound 877 isolated as a white solid foam (83.9%), was synthesized from compound 876 by following the procedure described in Example 229. ³¹P NMR (CD₃CN): δ 149.26, 149.63. ES MS: [M+H]⁺ 9934.1 (observed), 933.2 (calcd.).

### Example 247: Synthesis, cleavage, and analysis of oligonucleotide

Oligonucleotides were synthesized on an ABI 394 synthesizer using commercially available 5′-O-(4,4′-dimethoxytrityl)-2′-deoxy and 5′-O-(4,4′-dimethoxytrityl)-2′-O-methyl-3′-O-(2-cyanoethyl N, N-diisopropyl) phosphoramidites of thymidine, uridine, 4-N-acetyl cytidine, 6-N-benzoyl adenosine, and 2-N-isobutyryl guanosine using the standard solid-phase oligonucleotide synthesis. The removal of dimethoxy trityl group of 16 (or 17) takes longer treatment time than the regular machine recommended 3% TCA-CH₂Cl₂ cycle, hence the support was treated with 5% TCA-toluene to ensure complete removal of the dimethoxy trityl group prior to oligonucleotide synthesis. Three different activator (1 M 4,5-dicyanoimidazole+0.1 M N-methylimidazole in acetonitrile, 0.1 M 5-[3, 5-bis(trifluoromethyl)phenyl]-2H-tetrazole in acetonitrile (Activator 42®), 0.45 M 5-(benzylthio)-1H-tetrazole in acetonitrile) were tested and all were found to be equally effective. Oxidation solutions with different compositions of I₂, pyridine and water were tested (0.02 M I₂ in 89:10:1 THF:Water:Pyridine; 0.02 M I₂ in 88:10:2 THF: Pyridine: Water, and 0.05 M I₂ in 88:10:2 THF:Pyridine:Water). The oxidation solution of composition having 0.05 M 12 in 88:10:2 THF:Pyridine:Water, found to be most efficient. The sulfurization reaction was performed with 0.1 M DDTT (3-[[(dimethylamino)methylene]amino]-3H-1,2,4-dithiazole-5-thione) in anhydrous pyridine for a period of 3 minutes and was conducted prior to the capping step. Postsynthetically, all oligonucleotides were treated with 30% aq. diethylamine:acetonitrile (1:4) to remove the cyanoethyl protecting groups.

Cleavage of oligonucleotides from solid supports and the removal of base-protecting groups was carried out using aqueous ammonia and aqueous methylamine solutions of various concentrations. The timed deprotection samples were either evaporated in vacuo or, when they were used for kinetic studies, neutralized with 1.66 M aq. citric acid.

Analysis of oligonucleotides by RP-HPLC was performed on Phenomenex Kinetex 5 μm EVO C18 100 Å column or Luna 5 μm C18 (2) 100 Å column using 50 mM TRIS-HCl, pH 7.6 as Buffer A, 50 mM TRIS-HCl, pH 7.6: CH₃CN (20:80) as buffer B, column temperature of 60° C., and UV detection at 260 nm. Ion exchange-HPLC analysis was performed on Thermo Scientific DNAPac PA 200 columns using 20 mM Na₂PO₄: CH₃CN (90:10) pH 11.5 as Buffer A, 20 mM Na₂PO₄, 1 M NaBr:CH₃CN (90:10) pH 11.5 as buffer B, column temperature of 60° C., and UV detection at 260 nm.

### Example 248: Kinetic Studies

Study of kinetics of hydrolytic 3′-dephosphorylation of a T₁₀ oligonucleotide assembled on solid support 941 and of deprotection of N-isobutyryl-2′-deoxyguanosine residues in the protected oligonucleotides T₄dG^{ib}T₅ (42), T₃dG^{ib}₃ T₄ (43), (TdG^{ib})₃T₄ (44), and T₂dG^{ib}₆T₂ (45), was carried out in 1.37, 2.56, 3.42 M aq. MeNH₂ (7.5-, 4.0-, and 3.0-times dilution of 10.25 M aq., commercial 40% aq. methylamine) and at temperatures of 20.0, 30.0, 40.0, and 50.0° C. For each run, approximately 4 mg of a solid support-bound oligonucleotide was placed in an 8 mL glass vial equipped with a screw cap with a valve. This vial and a vial containing a methylamine solution were kept in a thermostat set at the desired temperature for at least 30 minutes. Approximately 4 mL of the methylamine solution was then transferred to the vial containing the oligo and the deprotection reaction started. Timed samples were taken out according to a schedule appropriate for the concentration of the methylamine solution and the temperature used and were neutralized immediately using 1.66 M aq. citric acid to pH 7.

The neutralized samples were analyzed by reverse-phase HPLC. HPLC analysis was performed on an Agilent 1260 HPLC system using Waters XBridge C18, 2.5 μm, 4.6×75 mm column, 100 mM HFIP, 5 mM TEA as Buffer A, acetonitrile as Buffer B, linear gradient from 1 to 14% B over 15 min at a flow rate of 0.45 mL×min⁻¹ or 1 to 12% B over 72 min at a flow rate of 0.675 mL×min⁻¹, and the column temperature of 50° C. The HPLC traces were recorded at 252 nm. To extract rate constants, the collected data were fitted to the equations (1) and (2) using solver module in Microsoft Excel.

Although the foregoing has been described in some detail by way of illustration and example for purposes of clarity and understanding, one of skill in the art will appreciate that certain changes and modifications can be practiced within the scope of the appended claims. All publications, patents, patent applications, and sequence accession numbers cited herein are hereby incorporated by reference in their entirety for all purposes.

INFORMAL SEQUENCE LISTING

SEQ ID NO: 1 (Synthetic Construct)
TTTTGTTTTT
SEQ ID NO: 2 (Synthetic Construct)
TTTGGGTTTT
SEQ ID NO: 3 (Synthetic Construct)
TGTGTGTTTT
SEQ ID NO: 4 (Synthetic Construct)
TTGGGGGGTT
SEQ ID NO: 5 (Synthetic Construct)
TTTTTTTTTT

1. A compound of Formula I 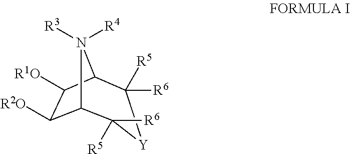
wherein:
R¹ and R² either form an orthoester function —C(CH₃)(OCH₃)— or one of R¹ and R² is hydrogen, a trityl protecting group or a derivative thereof, or a xanthenyl protecting group or a derivative thereof, and the other of R¹ and R² is an acetyl, a propionyl, a n-butyryl, a benzoyl, or L¹, wherein:
L¹ is a linking moiety —C(═O)—Z—(C═O)-A¹, wherein:
Z is selected from the group consisting of a covalent bond, a methylene group, —(CH₂)₂—, —(CH₂)—O—(CH₂)—, and —(CH₂)—O—C₆H₄—O—(CH₂)—;
A¹ is a hydroxy group, a salt of a hydroxy group and an inorganic cation or a tertiary amine, a covalent bond to SP¹, or —NH(CH₂)ₙ—OR⁷, wherein:
SP¹ is an oxygen, amino, an aminoalkyl or a hydroxyalkyl covalently attached to a solid phase material comprising a controlled pore glass, a magnetic controlled pore glass, a silica-containing particle, a styrene-containing polymer or copolymer, a divinylbenzene-containing polymer or copolymer, a copolymer of styrene and divinylbenzene, a controlled pore glass grafted with a styrene-containing polymer, a controlled pore glass grafted with a copolymer of styrene and divinylbenzene, a copolymer of styrene and divinylbenzene grafted with polyethylene glycol, or a flat glass surface;
n is an integer from 2 to 10;
R⁷ is hydrogen or PA, wherein: 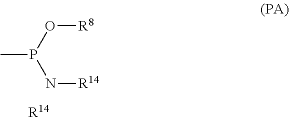
and wherein R⁸ is methyl or 2-cyanoethyl group and R¹⁴ is an alkyl, iso-alkyl, sec-alkyl, or tert-alkyl;
R³ is selected from the group consisting of methyl, ethyl, propyl, isopropyl, and benzyl;
R⁴ is selected from the group consisting of a lone electron pair, hydrogen, methyl, ethyl, propyl, isopropyl, and benzyl, wherein when R⁴ is other than a lone electron pair, N has a positive charge and forms a salt with a halide anion or with an intramolecular carboxylic functional group;
R⁵ and R⁶ are independently hydrogen or methyl;
Y is —(C═O)—, —CH(OR⁹)—, —CH(NR¹⁰R¹¹)—, or —[C(OR¹²)(OR¹³)]—, wherein:
R⁹ is hydrogen, methyl, ethyl, benzyl, acetyl, propionyl, butyryl, isobutyryl, pivaloyl, benzoyl, t-butyldimethylsilyl, triisopropylsilyl, (t-butyl) diphenylsilyl, —(C═O)A¹, L¹, or PA wherein:
m is an integer ranging from 2 to 10;
R¹⁰ is L¹, —(C═O)-A¹, or —(C═O)-W¹-(CH₂)ₚ—W², wherein:
W¹ is —(CH₂)—, —(NH)—, or —(NH)(C═O)—;
W² is a hydroxy, amino, —O—PA, (C═O)-A¹, or —[NH(C═O)]-A¹; and
p is an integer from 2 to 10;
R¹¹ is hydrogen, methyl, ethyl, or benzyl; and
R¹² and R¹³ together form ketal bridges —(CH₂)₂— or —CH₂ [C(CH₃)₂]—CH₂—;
provided one and only one of R¹, R², and Y is or includes L¹ or PA.

2. The compound of claim 1 wherein one of R¹ and R² is hydrogen, tris-(4-methoxyphenyl)methyl, bis-(4-methoxyphenyl)phenylmethyl, 9-phenylxanthen-9-yl, or 9-(4-methoxyphenyl)xanthen-9-yl, and the other of R¹ and R² is L¹ wherein:
A¹ is a hydroxy group, a salt of a hydroxy group and an inorganic cation or a tertiary amine, or a covalent bond to SP¹; and
(i) Y is —CH(OR⁹)—, wherein R⁹ is methyl, ethyl, benzyl, acetyl, propionyl, butyryl, isobutyryl, pivaloyl, benzoyl, t-butyldimethylsilyl, triisopropylsilyl, or phenyldimethylsilyl; or
(ii) Y is —[C(OR¹²)(OR¹³)]—.

3. The compound of claim 1 wherein one of R¹ and R² is hydrogen tris-(4-methoxyphenyl)methyl, or bis-(4-methoxyphenyl)phenylmethyl, and the other of R¹ and R² is acetyl, propionyl, n-butyryl, or benzoyl.

4. The compound of claim 3 wherein
(i) Y is —CH(OR⁹)—, wherein R⁹ is L¹, and wherein A¹ is a hydroxy group optionally forming a salt with an inorganic cation or a tertiary amine, or optionally forming a covalent bond to SP¹; or
(ii) Y is —CH(NR¹⁰R¹¹), wherein R¹¹ is hydrogen, methyl, ethyl, or benzyl, and
(a) R¹⁰ is L¹ wherein A¹ is a hydroxy group, a salt of a hydroxy group and an inorganic cation or a tertiary amine, or a covalent bond to SP¹; or
(b) R¹⁰ is —(C═O)-W¹-(CH₂)ₚ—W², wherein:
W² is an amino, —(C═O)-A¹, or —[NH(C═O)]-A¹ wherein A¹ is a hydroxy group, a salt of a hydroxy group and an inorganic cation or a tertiary amine, or a covalent bond to SP¹; and
p is an integer from 3 to 10.

5. The compound of claim 1 wherein one of R¹ and R² is tris-(4-methoxyphenyl)methyl or bis-(4-methoxyphenyl)phenylmethyl and the other of R¹ and R² is L¹ wherein:
(i) A¹ is-NH(CH₂)ₙ—OR⁷; and
(ii) Y is —CH(OR⁹), wherein R⁹ is methyl, ethyl, benzyl, acetyl, propionyl, butyryl, isobutyryl, pivaloyl, benzoyl, t-butyldimethylsilyl, triisopropylsilyl, or phenyldimethylsilyl.

6. The compound of claim 1 wherein one of R¹ and R² is tris-(4-methoxyphenyl)methyl or bis-(4-methoxyphenyl)phenylmethyl and the other of R¹ and R² is acetyl, propionyl, n-butyryl, or benzoyl and Y is —CH(OR⁹)—, wherein:
(i) R⁹ is PA, or
(ii) R⁹ is —(CH₂)ₘ—O-PA.

7. The compound of claim 1 wherein one of R¹ and R² is tris-(4-methoxyphenyl)methyl or bis-(4-methoxyphenyl)phenylmethyl, the other of R¹ and R² is acetyl, propionyl, n-butyryl, or benzoyl, and Y is —CH(NR¹⁰R¹¹), wherein R¹¹ is hydrogen, methyl, ethyl, or benzyl, wherein:
R¹⁰ is —(C═O)-W¹-(CH₂)ₚ—W², and wherein:
W¹ is —(CH₂)—, —(NH)—, or —(NH)(C═O)—, and
W² is hydroxy or O-PA.

8. The compound of claim 1 wherein R¹ and R² form an orthoester function —C(CH₃)(OCH₃)—, and wherein
(i) Y is —CH(OR⁹)—; and
(ii) R⁹ is hydrogen, PA, or —(CH₂)ₘ—O-PA.

9. The compound of claim 1, wherein the trityl protecting group or the derivative thereof or the xanthenyl protecting group or the derivative thereof is selected from the group consisting of 4-methoxytrityl, 4,4′-dimethoxytrityl, 4,4′,4″-trimethoxytrityl, 9-phenylxanthen-9-yl, and 9-(4-methoxyphenyl)xanthen-9-yl.

10. A method for functionalizing a solid phase material with a first monomeric subunit, comprising the steps of:
(a) providing a solid phase material-bound compound of Formula I 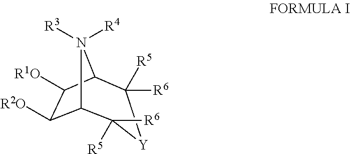
wherein:
R¹ and R² either form an orthoester function —C(CH₃)(OCH₃)— or one of R¹ and R² is hydrogen, a trityl protecting group or a derivative thereof, or a xanthenyl protecting group or a derivative thereof and the other of R¹ and R² is an acetyl, a propionyl, a n-butyryl, a benzoyl, L¹, wherein:
L¹ is a linking moiety —C(═O)—Z—(C═O)-A¹, wherein:
Z is selected from the group consisting of a covalent bond, a methylene group, —(CH₂)₂—, —(CH₂)—O—(CH₂)—, and —(CH₂)—O—C₆H₄—O—(CH₂)—;
A¹ is a covalent bond to SP¹ or —NH(CH₂)ₙ—OR⁷, wherein:
SP¹ is an oxygen, amino, an aminoalkyl or a hydroxyalkyl covalently attached to a solid phase material comprising a controlled pore glass, a magnetic controlled pore glass, a silica-containing particle, a styrene-containing polymer or copolymer, a divinylbenzene-containing polymer or copolymer, a copolymer of styrene and divinylbenzene, a controlled pore glass grafted with a styrene-containing polymer, a controlled pore glass grafted with a copolymer of styrene and divinylbenzene, a copolymer of styrene and divinylbenzene grafted with polyethylene glycol, and a flat glass surface;
n is an integer from 2 to 10;
R⁷ is PX, wherein: 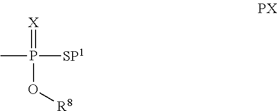
and wherein R⁸ is methyl or 2-cyanoethyl group and X is oxygen or sulfur;
R³ is selected from the group consisting of methyl, ethyl, propyl, isopropyl, and benzyl;
R⁴ is selected from the group consisting of a lone electron pair, hydrogen, methyl, ethyl, propyl, isopropyl, and benzyl, wherein when R⁴ is other than a lone electron pair, N has a positive charge and forms a salt with a halide anion or with an intramolecular carboxylic functional group;
R⁵ and R⁶ are independently hydrogen or methyl;
Y is —(C═O)—, —CH(OR⁹)—, —CH(NR¹⁰R¹¹), or —[C(OR¹²)(OR¹³)]—, wherein:
R⁹ is hydrogen, methyl, ethyl, benzyl, acetyl, propionyl, butyryl, isobutyryl, pivaloyl, benzoyl, t-butyldimethylsilyl, triisopropylsilyl, or (t-butyl) diphenylsilyl group, —(C═O)A¹ L¹, or PX, wherein:
m is an integer ranging from 2 to 10;
R¹⁰ is L¹, —(C═O)-A¹, or —(C═O)-W¹ (CH₂)ₚ—W², wherein:
W¹ is —(CH₂)—, —(NH)—, or —(NH)—(C═O)—;
W² is —O—PX, or —[NH(C═O)]-A¹; and
p is an integer ranging from 2 to 10;
R¹¹ is hydrogen, methyl, ethyl, or benzyl; and
R¹² and R₁₃ together form ketal bridges —(CH₂)₂— or —CH₂—[C(CH₃)₂]—CH₂—;
provided one and only one of R¹, R², and Y is or includes L¹ or PX;
(b) selectively removing one of the protecting groups of Formula I to form a reactive hydroxyl group;
(c) providing a first monomeric subunit comprising an activated phosphorus group and a protected hydroxy group, and reacting the activated phosphorus group of the first monomeric subunit with the reactive hydroxyl group of the compound of Formula I to form a monomer-functionalized solid support comprising a phosphite group;
(d) treating said monomer-functionalized solid support with a capping agent and/or treating said monomer-functionalized solid support with an oxidizing solution or a sulfurizing agent to convert the phosphite triester group to a phosphotriester or phosphothioate triester, thereby forming an oxidized or sulfurized functionalized solid support;
(e) optionally repeating steps (b), (c), and (d) one or more times for the oxidized or sulfurized functionalized solid support to form an oligomeric-functionalized solid support, wherein the monomeric subunit is the same or different each time steps (b), (c), and (d) are repeated.

11. The method of claim 10, further comprising deprotecting the oligomeric-functionalized solid support of step (e) and cleaving the oligomeric-functionalized solid support to form an oligomeric compound separate from the solid phase material, wherein the cleaving forms a terminal hydroxy group on the oligomeric compound at the site of cleavage.

12. The method of claim 10, wherein one of R¹ or R² is L¹.

13. The method of claim 10, wherein R⁷ is PX.

14. The method of claim 10, wherein the activated phosphorus group comprises a phosphoramidite, an H-phosphonate, or a phosphate triester.

15. The method of claim 11, wherein the oligomeric compound is an oligonucleotide, optionally comprising unnatural sugar-modified nucleotide residues, unnatural base-modified nucleotide residues, or non-nucleotide monomeric units.
